(19) United States
(12) Patent Application Publication
(10) Pub. No.: US 2024/0251767 A1
(43) Pub. Date: Aug. 1, 2024

(54) ENGINEERED NON-HUMAN ANIMALS FOR PRODUCING ANTIBODIES

(71) Applicant: Leveragen, Inc., Worcester, MA (US)

(72) Inventor: Weisheng Victor CHEN, Hopkinton, MA (US)

(21) Appl. No.: 18/289,508

(22) PCT Filed: May 5, 2022
(86) PCT No.: PCT/US22/27946

§ 371 Date: Nov. 3, 2023

Related U.S. Application Data
(60) Provisional application No. 63/184,385, filed on May 5, 2021.
(60) Provisional application No. 63/184,384, filed on May 5, 2021.

(51) Int. Cl. A01K 67/0278; C07K 16/10; C12N 15/85

(52) U.S. Cl. CPC A01K 67/0278; C07K 16/1003; C12N 15/8509; A01K 2207/15; A01K 2217/075; A01K 2217/15; A01K 2227/105; A01K 2267/01; C07K 2317/10; C07K 2317/24; C07K 2317/522; C07K 2317/524; C07K 2317/569; C07K 2317/92; C12N 2015/8518

(57) ABSTRACT

This document provides genetically modified animals (e.g., mice), humanized heavy chain antibodies, humanized nanobodies, and methods of making and using the same. For example, genetically engineered non-human animals (e.g., genetically engineered mice) that can be designed to produce heavy chain antibodies that can be used to generate a single domain antibody or nanobody are provided.

### CROSS-REFERENCE TO RELATED APPLICATIONS

This application claims the benefit of U.S. Provisional Application Ser. No. 63/184,384, filed on May 5, 2021, and U.S. Provisional Application Ser. No. 63/184,385, filed on May 5, 2021. The disclosure of the prior applications is considered part of, and is incorporated in its entirety by reference in, the disclosure of this application.

### BACKGROUND

### 1. Technical Field

This document relates to methods and materials involved in producing antibodies (e.g., heavy chain antibodies and/or single domain antibodies also known as nanobodies).

For example, genetically engineered non-human animals (e.g., genetically engineered mice) having the ability to produce antibodies (e.g., heavy chain antibodies such as heavy chain antibodies lacking CH1 domains and light chains) are provided. In some cases, a heavy chain antibody obtained as described herein can be used to generate a single domain antibody or nanobody.

### 2. Background Information

Conventional IgG antibodies consist of four polypeptides: two pairs of identical heavy and light chains. The heavy chains of IgGs contain one variable domain (VH) and three constant domains (CH1, CH2 and CH3) with the two chains connected via disulfide bonds at the hinge region (H) between CH1 and CH2. The two light chains contain one variable domain (VL) and one constant (CL) domain with the CL domain connected to the CH1 domain of the heavy chains via disulfide bonds to form the tetrameric IgGs. The two antibody arms (Fabs) made up of VH-CH1 and VL-CL can independently bind antigens, and the constant region (Fc) is responsible for effector functions. Generation of antibodies starts with expression of B cell receptor (BCR) in pre-B cells by the assembly of the V (variable), D (diversity), and J (joining) gene segments via VDJ recombination of the immunoglobulin heavy chain gene (IgH) to generate diverse VHs. VDJ is spliced into the constant exons of IgM, which then associate with a surrogate light chain A6 and VpreB on the cell surface to form the pre-BCR. This is followed by VJ recombination of the immunoglobulin light chain genes (Igκ and IgL, both lacking the D genes) and the production of diverse VLs with CLs. Pairing of the light chain with the heavy chain of IgM results in IgM expression as a complete BCR on immature B cells. While V(D)J recombination takes place on both alleles of the heavy chain and light chain loci, allelic exclusion ensures the expression of only one functional heavy chain and one functional light chain from one of the two alleles in a single B cell. B cells carrying successful recombination events then undergo somatic hypermutation, antigen selection, affinity maturation, and class switch recombination to express different isotypes of antibodies (IgG, IgE, and IgA).

In camelids, a subset of IgGs composed of only two identical heavy chains bearing a variable domain (Variable Heavy Homodimer, VHH), yet lacking the CH1 domain and the associated light chains have been identified. These heavy chain-only antibodies (HCAbs) result from splice site mutations in the heavy chain gene that abolish the CH1 exon, which encodes the CH1 domain of the constant region that normally binds the CL of the light chains. Similar HCAbs devoid of light chains are also found in cartilaginous fish (Immunoglobulin New Antigen Receptors, IgNARs). The variable domains in HCAbs-VHHs in camelids and VNARs in cartilaginous fish—can function as independent antigen-binding units and have comparable binding affinity to conventional antibodies. These single domain antibodies (sdAbs) are the smallest antigen binding antibody fragments and are thus sometimes referred to as nanobodies (Nbs). A number of unique properties of sdAbs including their small sizes (11-15 KDa as compared to 150 KDa of tetrameric antibodies), strict monomericity, high solubility, efficient folding/refolding, superior stability, unparalleled target accessibility, effective tissue penetration, fast blood clearance, excellent manufacturability, and low cost of production make them compelling candidates for developing novel therapeutic and diagnostic agents. One of the most advantageous properties of sdAbs over conventional antibodies is their modularity, which is a key property for easier engineering of multimeric and multi-specific biologics.

Variable domains (VH and VL) derived from human scaffolds have been produced in synthetic sdAb display libraries and tested against a number of targets in vitro (Belanger et al., Protein Eng. Des. Sel., (34):gzab012 (2021)). Contrary to naturally occurring sdAbs obtained from immunized animals that have high affinities as a result of somatic hypermutation, sdAbs derived from non-immune display libraries are usually of low affinity and tend to aggregate, and often require further mutations for improved affinity and function.

A solution to both problems is to genetically engineer mice that produce humanized HCAbs, which can be immunized with targets of interest to isolate functional human sdAbs of high solubility and affinity resulting from antigen selection and affinity maturation in vivo.

### SUMMARY

This document relates to engineered non-human animals (e.g., mice) that produce single domain antibodies or nanobodies (e.g., mouse single domain antibodies or mouse nanobodies, or humanized single domain antibodies or humanized nanobodies) as well as methods of making such engineered non-human animals (e.g., mice) and methods of using such engineered non-human animals (e.g., mice). For example, this document provides genetically engineered non-human animals (e.g., genetically engineered mice) that can be designed to produce heavy chain antibodies that can be used to generate a single domain antibody or nanobody. In some cases, a genetically engineered non-human animal (e.g., a genetically engineered mouse) can be designed to produce heavy chain antibodies (e.g., fully mouse heavy chain IgG antibodies) that lack CH1 domains and light chains and that can be used to generate single domain antibodies or nanobodies (e.g., fully mouse single domain antibodies or fully mouse nanobody). See, e.g., FIG. 1. In some cases, a genetically engineered non-human animal (e.g., a genetically engineered mouse) can be designed to produce chimeric heavy chain antibodies (e.g., human-mouse chimeric heavy chain IgG antibodies) that lack CH1 domains and light chains and that can be used to generate single domain antibodies or nanobodies that also can be chimeric or that can be fully of one species.

For example, a genetically engineered mouse can be designed to produce human-mouse chimeric heavy chain IgG antibodies that lack CH1 domains and light chains while having a human variable domain and mouse constant domains. Such human-mouse chimeric heavy chain IgG antibodies obtained from such a mouse can be used to generate fully human single domain antibodies or human nanobodies. See, e.g., FIG. 6. The compositions described herein (e.g., compositions containing one or more antibodies generated from an engineered non-human animal (e.g., mouse) provided herein) can be used to treat or prevent a disease or disorder, for example, an inflammatory disease.

As described herein, genetically engineered non-human animals (e.g., genetically engineered mice) can be designed to produce heavy chain antibodies (e.g., mouse heavy chain antibodies or chimeric heavy chain antibodies such as human-mouse chimeric heavy chain antibodies, bovine-human-mouse chimeric heavy chain antibodies, alpaca-human-mouse chimeric heavy chain antibodies, or shark-human-mouse chimeric heavy chain antibodies) that lack CH1 domains and light chains. Such heavy chain antibodies can be used to generate single domain antibodies or nanobodies (e.g., mouse single domain antibodies, also referred to herein as mouse nanobodies, non-mouse single domain antibodies, also referred to herein as non-mouse nanobodies, humanized single domain antibodies, also referred to herein as humanized nanobodies, human single domain antibodies, also referred to herein as human nanobodies, bovine-human chimeric single domain antibodies, also referred to herein as bovine-human chimeric nanobodies, alpaca-human chimeric single domain antibodies, also referred to herein as alpaca-human chimeric nanobodies, or shark-human chimeric single domain antibodies, also referred to herein as shark-human chimeric nanobodies).

As also described herein, engineered non-human animals (e.g., mice) provided herein can be used to obtain heavy chain antibodies (e.g., mouse heavy chain antibodies or chimeric heavy chain antibodies) that can be used to generate single domain antibodies (e.g., mouse single domain antibodies or non-mouse single domain antibodies such as humanized single domain antibodies or human single domain antibodies

In one embodiment, this document provides a genetically engineered mouse comprising a germline modification comprising a deletion of a nucleic acid sequence comprising one or more heavy-chain C-region genes; wherein the mouse expresses an IgG heavy chain antibody and secretes the IgG heavy chain antibody into its serum.

In some embodiments, the one or more heavy-chain C-region genes comprises an IgM C-region gene (Cμ), an IgD C-region gene (Cδ), an IgE C-region gene (Cε), an IgG3 C-region gene (Cγ3), an IgG2b C-region gene (Cγ2b), an IgG2c C-region gene (Cγ2c), or a combination thereof.

In some embodiments, the genetically engineered mouse further comprises a deletion of a nucleic acid sequence encoding a CH1 domain of an IgG1 C-region gene (Cγ1). In some embodiments, the deletion of the nucleic acid sequence encoding the CH1 domain of the IgG1 C-region gene comprises exon 1.

In some embodiments, the germline modification further comprises a native nucleic acid sequence encoding a hinge (H) domain, heavy-chain CH2 domain, a heavy-chain CH3 domain or a combination thereof.

In some embodiments, the germline modification further comprises a native nucleic acid sequence comprising an endogenous enhancer. In some embodiments, the enhancer comprises Eμ, 3′RR, 3′γ1E, 5′hsR1, or a combination thereof.

In some embodiments, the germline modification further comprises a native nucleic acid sequence comprising a switch tandem repeat element (Sμ) and Iμ promoter, wherein Iμ drives constitutive expression of IgG1 truncated for CH1 domain (IgG1ΔCH1).

In some embodiments, the IgG heavy chain antibody comprises an IgG1 heavy chain antibody. In some embodiments, the IgG1 heavy chain antibody is a IgG1ΔCH1 protein. In some embodiments, the IgG heavy chain antibody lacks a light chain. In some embodiments, the IgG heavy chain antibody comprises a hinge domain, CH2 domain, a CH3 domain, or a combination thereof.

In some embodiments, the mouse does not express a wild-type IgM protein, a wild-type IgD protein, a wild-type IgE protein, a wild-type IgG3 protein, or a combination thereof.

In some embodiments, the mouse does not express a wild-type IgA protein, a wild-type IgG2b protein, a wild-type IgG2c protein, or a combination thereof.

In another embodiment, this document provides an engineered non-human animal comprising a germline genome comprising an engineered immunoglobulin heavy chain (IgH) allele at an endogenous IgH locus; wherein the engineered IgH allele lacks an endogenous heavy-chain C region gene; and wherein the endogenous heavy-chain C region gene comprises Cμ, Cδ, Cε, Cγ3, Cγ2b, Cγ2c, or a combination thereof.

In some embodiments, the IgH allele comprises a deletion of a nucleic acid sequence encoding a CH1 domain of an IgG1 C-region gene (Cγ1). In some embodiments, the CH1 domain of the IgG1 C-region gene comprises exon 1.

In some embodiments, the IgH locus comprises a native nucleic acid sequence encoding a hinge (H) domain, heavy-chain CH2 domain, a heavy-chain CH3 domain, or a combination thereof.

In some embodiments, the IgH locus comprises a native nucleic acid sequence comprising an endogenous enhancer. In some embodiments, the enhancer comprises Eμ, 3′RR, 3′γ1E, 5′hsR1, or a combination thereof.

In some embodiments, the IgH locus comprises a native nucleic acid sequence comprising a switch tandem repeat element (Sμ) and Iμ promoter, wherein Iμ drives constitutive expression of IgG1 truncated for CH1 domain (IgG1ΔCH1).

In some embodiments, the non-human animal expresses an IgG heavy chain antibody.

In some embodiments, the IgG heavy chain antibody comprises an IgG1 heavy chain antibody.

In some embodiments, the IgG1 heavy chain antibody is a IgG1ΔCH1 protein.

In some embodiments, the IgG heavy chain antibody lacks a light chain.

In some embodiments, the IgG heavy chain antibody comprises a hinge domain, a CH2 domain, a CH3 domain, or a combination thereof.

In some embodiments, the non-human animal does not express wild-type IgM protein, a wild-type IgD protein, a wild-type IgE protein, a wild-type IgG3 protein, a wild-type IgA protein, a wild-type IgG2b protein, a wild-type IgG2c protein, or a combination thereof.

In some embodiments, the IgH locus comprises endogenous V, D, or J genes.

In some embodiments, the engineered non-human animal is homozygous for the engineered IgH allele.

In some embodiments, the endogenous IgH locus does not comprise an exogenous nucleic acid sequence.

In some embodiments, the endogenous IgH locus comprises an exogenous nucleic acid sequence. In some embodiments, the exogenous nucleic acid sequence comprises a bar code.

In another embodiment, this document provides an engineered non-human animal, wherein the non-human animal is a mammal. In some embodiments, the mammal is a mouse.

In another embodiment, this document provides a method of producing a genetically modified non-human animal capable of producing a heavy-chain antibody comprising (a) deleting an endogenous nucleic acid sequence comprising one or more heavy-chain C-region genes from an endogenous immunoglobulin heavy chain locus in a stem cell of a non-human animal, (b) implanting the stem cell into a blastocyst, (c) implanting the blastocyst into a pseudo-pregnant mouse to obtain a chimeric mouse, (d) crossing the chimeric mouse to a wild-type mouse to produce offspring, (e) screening the offspring for heterozygosity, and (f) identifying a founder mouse carrying a deletion of one or more heavy-chain C-region genes, and wherein said non-human animal is capable of producing a heavy chain antibody.

In some embodiments, the stem cell is an embryonic stem cell.

In some embodiments, the one or more heavy-chain C-region genes comprises Cμ, Cδ, Cγ3, Cγ2b, Cγ2c, Cε, or a combination thereof.

In some embodiments, the method further comprises deleting a nucleic acid sequence encoding a CH1 domain of an IgG1 C-region gene and CH1 exon of Cγ1. In some embodiments, the deletion of the nucleic acid sequence encoding the CH1 domain of the IgG1 C-region gene comprises exon 1.

In some embodiments, the method further comprises preserving a native nucleic acid sequence encoding a hinge (H) domain, heavy-chain CH2 domain, and a heavy-chain CH3 domain of IgG1 (Cγ1), or a combination thereof.

In some embodiments, the method further comprises preserving a native nucleic acid sequence comprising an endogenous enhancer. In some embodiments, the enhancer comprises Eμ, 3′RR, 3′γ1E, 5′hsR1, or a combination thereof.

In some embodiments, the method further comprises preserving a native nucleic acid sequence comprising a switch tandem repeat element (Sμ) and Iμ promoter, wherein Iμ drives constitutive expression of IgG1 truncated for CH1 domain (IgG1ΔCH1).

In some embodiments, the heavy chain antibody is an IgG heavy chain antibody. In some embodiments, the IgG heavy chain antibody comprises an IgG1 heavy chain antibody.

In some embodiments, the IgG1 heavy chain antibody is a IgG1ΔCH1 protein.

In some embodiments, the IgG1 heavy chain antibody lacks a light chain.

In some embodiments, the IgG1 heavy chain antibody comprises a hinge domain, CH2 domain, a CH3 domain, or a combination thereof.

In some embodiments, the non-human animal does not express a wild-type IgM protein, a wild-type IgD protein, a wild-type IgE protein, a wild-type IgG3 protein, a wild-type IgA protein, a wild-type IgG2b protein, a wild-type IgG2c protein, or a combination thereof.

In some embodiments, the non-human animal is a mammal. In some embodiments, the mammal is a mouse.

In some embodiments, deleting an endogenous nucleic acid sequence comprising one or more heavy-chain C-region genes comprises CRISPR/Cas9 genome editing.

In some embodiments, the genetically modified non-human animal is fertile. In some embodiments, the genetically modified non-human animal has normal B cell development and maturation.

In some embodiments, the genetically modified non-human animal does not express a wild-type IgM protein, a wild-type IgD protein, a wild-type IgE protein, a wild-type IgG3 protein, a wild-type IgA protein, a wild-type IgG2b protein, a wild-type IgG2c protein, or a combination thereof.

In another embodiment, this document provides a method of producing a soluble heavy-chain antibody in the engineered non-human animal comprising (a) administering to the non-human animal an antigen, (b) isolating one or more B cells from the non-human animal, (c) isolating mRNA from the one or more B cells, (d) sequencing the mRNA, (e) identifying clonal type based on the mRNA sequence, and (f) phylogenetic analysis of the clonal type; thereby producing a soluble heavy-chain antibody.

In some embodiments, the non-human animal is a mammal. In some embodiments, the mammal is a mouse.

In another embodiment, this document provides a method of producing a single domain antibody (sdAb) identified from the engineered non-human animal comprising expressing a nucleic acid sequence encoding a heavy chain variable (VH) domain comprising a V, D and J in a cell, wherein the cell produces the heavy chain variable domain, and isolating the heavy chain variable domain from a sample thereby producing the single domain antibody. In some embodiments, the single domain antibody is a murine single domain antibody.

In some embodiments, the single domain antibody is an IgG1 single domain antibody derived from an IgG1 heavy chain antibody. In some embodiments, the IgG1 single domain antibody is a IgG1ΔCH1 nanobody derived from a IgG1ΔCH1 heavy chain antibody.

In another embodiment, this document provides a genetically engineered mouse comprising a germline modification comprising a deletion of a nucleic acid sequence comprising one or more heavy-chain C-region genes; wherein the mouse expresses a humanized IgG heavy chain antibody and secretes the humanized IgG heavy chain antibody into its serum.

In some embodiments, the one or more heavy-chain C-region genes is an IgM C-region gene (Cμ), an IgD C-region gene (Cδ), an IgE C-region gene (Cε), an IgG3 C-region gene (Cγ3), an IgG2b C-region gene (Cγ2b), an IgG2c C-region gene (Cγ2c), or a combination thereof.

In some embodiments, the genetically engineered mouse further comprises a deletion of a nucleic acid sequence encoding a CH1 domain of an IgG1 C-region gene (Cγ1). In some embodiments, the deletion of the nucleic acid sequence encoding the CH1 domain of the IgG1 C-region gene comprises exon 1.

In some embodiments, the germline modification further comprises a native nucleic acid sequence encoding a hinge (H) domain, heavy-chain CH2 domain, and a heavy-chain CH3 domain of IgG1 (Cγ1) or a combination thereof.

In some embodiments, the germline modification further comprises a native nucleic acid sequence comprising an endogenous enhancer. In some embodiments, the enhancer is Ep, 3′RR, 3′γ1E, 5′hsR1, or a combination thereof.

In some embodiments, the germline modification further comprises a native nucleic acid sequence comprising a switch tandem repeat element (Sμ) and Iμ promoter, wherein Iμ drives constitutive expression of IgG1 truncated for CH1 domain (IgG1ΔCH1).

In some embodiments, the humanized IgG heavy chain antibody comprises a humanized IgG1 heavy chain antibody. In some embodiments, the humanized IgG1 heavy chain antibody is a IgG1ΔCH1 protein.

In some embodiments, the humanized IgG heavy chain antibody lacks a light chain.

In some embodiments, the humanized IgG heavy chain antibody comprises a hinge domain, CH2 domain, and CH3 domain of IgG1 (Cγ1), or a combination thereof.

In some embodiments, the mouse does not express a wild-type IgM protein, a wild-type IgD protein, a wild-type IgE protein, a wild-type IgG3 protein, or a combination thereof.

In some embodiments, the mouse does not express a wild-type IgA protein, a wild-type IgG2b protein, a wild-type IgG2c protein, or a combination thereof.

In another embodiment, this document provides an engineered non-human animal comprising a germline genome comprising an engineered immunoglobulin heavy chain (IgH) allele at an endogenous IgH locus; wherein the engineered IgH allele lacks an endogenous heavy-chain C region gene; and wherein the endogenous heavy-chain C region gene comprises Cμ, Cδ, Cε, Cγ3, Cγ2b, Cγ2c, or a combination thereof.

In some embodiments, the IgH allele comprises a deletion of a nucleic acid sequence encoding a CH1 domain of an IgG1 C-region gene (Cγ1). In some embodiments, the CH1 domain of the IgG1 C-region gene comprises exon 1.

In some embodiments, the IgH locus comprises a native nucleic acid sequence encoding a hinge (H) domain, heavy-chain CH2 domain, and a heavy-chain CH3 domain of IgG1 (Cγ1), or a combination thereof.

In some embodiments, the IgH locus comprises a native nucleic acid sequence comprising an endogenous enhancer. In some embodiments, the enhancer is Eμ, 3′RR, 3′γ1E, 5′hsR1, or a combination thereof.

In some embodiments, the IgH locus comprises a native nucleic acid sequence comprising a switch tandem repeat element (Sμ) and Iμ promoter, wherein Iμ drives constitutive expression of IgG1 truncated for CH1 domain (IgG1ΔCH1).

In some embodiments, the non-human animal expresses a humanized IgG heavy chain antibody. In some embodiments, the humanized IgG1 heavy chain antibody comprises a humanized IgG1 heavy chain antibody. In some embodiments, the humanized IgG1 heavy chain antibody is a IgG1ΔCH1 protein.

In some embodiments, the humanized IgG1 heavy chain antibody lacks a light chain.

In some embodiments, the humanized IgG1 heavy chain antibody comprises a hinge domain, a CH2 domain, a CH3 domain, or a combination thereof.

In some embodiments, the non-human animal does not express wild-type IgM protein, a wild-type IgD protein, a wild-type IgE protein, a wild-type IgG3 protein, or a combination thereof. In some embodiments, the non-human animal does not express a wild-type IgA protein, a wild-type IgG2b protein, a wild-type IgG2c protein, or a combination thereof. In some embodiments, the IgH locus comprises human V, D, or J genes.

In some embodiments, the engineered non-human animal is homozygous for the engineered IgH allele.

In some embodiments, the endogenous IgH locus comprises an exogenous nucleic acid sequence.

In some embodiments, the exogenous nucleic acid sequence comprises one or more human V_{H} gene segments, one or more human D_{H} gene segments, and one or more J_{H} gene segments. In some embodiments, the exogenous nucleic acid sequence comprises two or more human V_{H} gene segments, two or more human D_{H} gene segments, and two or more J_{H} gene segments.

In some embodiments, the exogenous nucleic acid sequence comprises 1, 2, 3, 4, 5, 6, 7, 8,9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65 human V_{H} gene segments. In some embodiments, the exogenous nucleic acid sequence comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, or 126 human V_{H} gene segments. In some embodiments, the exogenous nucleic acid sequence comprises 1-5, 6-10, 11-15, 16-20, 21-25, 26-30, 31-35, 36-40, 41-45, 46-50, 51-55, 56-60, or 60-65 human VH gene segments. In some embodiments, the exogenous nucleic acid sequence comprises 1-5, 6-10, 11-15, 16-20, 21-25, 26-30, 31-35, 36-40, 41-45, 46-50, 51-55, 56-60, 60-65, 66-70, 71-75, 76-80, 81-85, 86-90, 91-95, 96-100, 101-105, 106-110, 111-115, 116-120, or 121-126 human V_{H} gene segments. In some embodiments, the exogenous nucleic acid sequence comprises substantially all human V_{H} gene segments. In some embodiments, the exogenous nucleic acid sequence comprises about 10, about 20, about 30, about 40, about 50, or about 60 human V_{H} gene segments. In some embodiments, the exogenous nucleic acid sequence comprises about 10, about 20, about 30, about 40, about 50, about 60, about 70, about 80, about 90, about 100, about 110, or about 120 human V_{H} gene segments. In some embodiments, the exogenous nucleic acid sequence comprises more than 1, more than 10, more than 20, more than 30, more than 40, more than 50, or more than 60 human VH gene segments. In some embodiments, the exogenous nucleic acid sequence comprises more than 1, more than 10, more than 20, more than 30, more than 40, more than 50, more than 60, more than 70, more than 80, more than 90, more than 100, more than 110, or more than 120 human V_{H} gene segments. In some embodiments, the exogenous nucleic acid sequence comprises 65 human VH gene segments. In some embodiments, the exogenous nucleic acid sequence comprises 126 human VH gene segments.

In some embodiments, the exogenous nucleic acid sequence comprises 1, 2, 3, 4, 5, 6, 7, 8, 9, 10, 11, 12, 13, 14, 15, 16, 17, 18, 19, 20, 21, 22, 23, 24, 25, 26, or 27 human D_{H} gene segments. In some embodiments, the exogenous nucleic acid sequence comprises 1-5, 6-10, 11-15, 16-20, 21-25, or 26-27 human D_{H} gene segments. In some embodiments, the exogenous nucleic acid sequence comprises substantially all human DH gene segments. In some embodiments, the exogenous nucleic acid sequence comprises about 5, about 10, about 15, about 20, or about 25 human D_{H} gene segments. In some embodiments, the exogenous nucleic acid sequence comprises more than 1, more than 5, more than 10, more than 15, more than 20, or more than 25 human D_{H} gene segments. In some embodiments, the exogenous nucleic acid sequence comprises 27 human D_{H} gene segments.

In some embodiments, the exogenous nucleic acid sequence comprises 1, 2, 3, 4, 5, or 6 human J_{H} gene segments. In some embodiments, the exogenous nucleic acid sequence comprises 1, 2, 3, 4, 5, 6, 7, 8, or 9 human J_{H} gene segments. In some embodiments, the exogenous nucleic acid sequence comprises 1-6 human J_{H} gene segments. In some embodiments, the exogenous nucleic acid sequence comprises 1-9 human J_{H} gene segments.

In some embodiments, the exogenous nucleic acid sequence comprises substantially all human J_{H} gene segments. In some embodiments, the exogenous nucleic acid sequence comprises about 5 human J_{H} gene segments. In some embodiments, the exogenous nucleic acid sequence comprises about 9 human J_{H} gene segments. In some embodiments, the exogenous nucleic acid sequence comprises more than 1, more than 2, more than 3, more than 4, or more than 5 human J_{H} gene segments. In some embodiments, the exogenous nucleic acid sequence comprises more than 1, more than 2, more than 3, more than 4, more than 5, more than 6, more than 7, or more than 8 human J_{H} gene segments. In some embodiments, the exogenous nucleic acid sequence comprises 6 J_{H} gene segments.

In some embodiments, the exogenous nucleic acid sequence comprises 65 human V_{H} gene segments, 27 human D_{H} gene segments, and 6 J_{H} gene segments.

In some embodiments, the exogenous nucleic acid sequence comprises 127 human V_{H} gene segments, 27 human DH gene segments, and 9 J_{H} gene segments.

In some embodiments, the exogenous nucleic acid sequence comprises a bar code.

In some embodiments, the non-human animal is a mammal. In some embodiments, the mammal is a mouse or a rat.

In another embodiment, this document provides a method of producing a genetically modified non-human animal capable of producing a humanized heavy-chain antibody comprising (a) deleting an endogenous nucleic acid sequence comprising one or more heavy-chain C-region genes from an endogenous immunoglobulin heavy chain locus in a stem cell of a non-human animal; (b) implanting the stem cell into a blastocyst; (c) implanting the blastocyst into a pseudo-pregnant mouse to obtain a chimeric mouse; (d) crossing the chimeric mouse to a wild-type mouse to produce offspring; (e) screening the offspring for heterozygosity; and (f) identifying a founder mouse carrying a deletion of one or more heavy-chain C-region genes; wherein said non-human animal is capable of producing a humanized heavy chain antibody.

In some embodiments, the stem cell is an embryonic stem cell.

In some embodiments, the one or more heavy-chain C-region genes comprises Cμ, Cδ, Cγ3, Cγ2b, Cγ2c, Cε, or a combination thereof.

In some embodiments, the method further comprises deleting a nucleic acid sequence encoding a CH1 domain of an IgG1 C-region gene. In some embodiments, the deletion of the nucleic acid sequence encoding the CH1 domain of the IgG1 C-region gene comprises exon 1.

In some embodiments, the method further comprises preserving a native nucleic acid sequence encoding a hinge (H) domain, heavy-chain CH2 domain, a heavy-chain CH3 domain of IgG1 (Cγ1), or a combination thereof.

In some embodiments, the method further comprises preserving a native nucleic acid sequence comprising an endogenous enhancer. In some embodiments, the enhancer is Eμ, 3′RR, 3′γ1E, 5′hsR1, or a combination thereof.

In some embodiments, the method further comprises preserving a native nucleic acid sequence comprising a switch tandem repeat element (Sμ) and Iμ promoter, wherein Iμ drives constitutive expression of IgG1 truncated for CH1 domain (IgG1ΔCH1).

In some embodiments, the humanized heavy chain antibody is a humanized IgG1 heavy chain antibody. In some embodiments, the humanized IgG heavy chain antibody comprises a humanized IgG1 heavy chain antibody. In some embodiments, the IgG1 heavy chain antibody is a IgG1ΔCH1 protein.

In some embodiments, the humanized IgG1 heavy chain antibody lacks a light chain.

In some embodiments, the humanized IgG1 heavy chain antibody comprises a hinge domain, CH2 domain, a CH3 domain, or a combination thereof.

In some embodiments, the non-human animal does not express a wild-type IgM protein, a wild-type IgD protein, a wild-type IgE protein, a wild-type IgG3 protein, a wild-type IgA protein, a wild-type IgG2b protein, a wild-type IgG2c protein, or a combination thereof.

In some embodiments, the non-human animal is a mammal. In some embodiments, the mammal is a mouse.

In some embodiments, deleting an endogenous nucleic acid sequence comprising one or more heavy-chain C-region genes comprises CRISPR/Cas9 genome editing.

In the embodiments, the genetically modified non-human animal is fertile.

In some embodiments, the genetically modified non-human animal has substantially normal B cell development and maturation.

In some embodiments, the genetically modified non-human animal does not express a wild-type IgM protein, a wild-type IgD protein, a wild-type IgE protein, a wild-type IgG3 protein, a wild-type IgA protein, a wild-type IgG2b protein, a wild-type IgG2c protein, or a combination thereof.

In another embodiment, this document provides a method of producing a soluble humanized heavy-chain antibody in the engineered non-human animal comprising (a) administering to the non-human animal an antigen; (b) isolating one or more B cells from the non-human animal; (c) isolating mRNA from the one or more B cells; (d) sequencing the mRNA; (e) identifying clonal type based on the mRNA sequence; and (f) phylogenetic analysis of the clonal type; thereby producing a soluble humanized heavy-chain antibody.

In some embodiments, the non-human animal is a mammal. In some embodiments, the mammal is a mouse or a rat.

In another embodiment, this document provides a method of producing a humanized single domain antibody (sdAb) identified from the engineered non-human animal comprising expressing a nucleic acid sequence encoding a human heavy chain variable (VH) domain comprising a V, a D and a J in a cell, wherein the cell produces the human heavy chain variable domain; and isolating the human heavy chain variable domain from a sample thereby producing the single domain antibody.

In some embodiments, the single domain antibody is a human single domain antibody. In some embodiments, the single domain antibody is an IgG1 single domain antibody. In some embodiments, the IgG1 single domain antibody is a IgG1ΔCH1 nanobody.

In some embodiments, the single domain antibody lacks a light chain.

In some embodiments, the single domain antibody lacks a hinge domain, a CH2 domain, a CH3 domain or a combination thereof.

In some embodiments, the cell is a bacterial cell or a human cell.

In another aspect, this document features a non-human animal, wherein the genome of the non-human animal comprises an immunoglobulin heavy chain (IgH) allele, wherein the IgH allele (or the genome) comprises an endogenous nucleic acid encoding a CH2 or CH3 domain of an IgG subclass, wherein the IgH allele (or the genome) lacks nucleic acid encoding at least a portion of an endogenous CH1 domain of the IgG subclass, and wherein the IgH allele (or the genome) lacks endogenous nucleic acid encoding at least a portion of an IgM constant domain, endogenous nucleic acid encoding at least a portion of an IgD constant domain, endogenous nucleic acid encoding at least a portion of an IgE constant domain, or endogenous nucleic acid encoding at least a portion of an IgA constant domain. The IgH allele (or the genome) of the non-human animal can comprise endogenous nucleic acid encoding the CH2 domain and the CH3 domain of the IgG subclass. The IgH allele (or the genome) of the non-human animal can comprise endogenous nucleic acid encoding a hinge domain of the IgG subclass. The IgG subclass can be an IgG2 subclass. The IgG subclass can be an IgG2a, IgG2b, IgG2c, IgG3, or IgG4 subclass. The IgG subclass can be an IgG1 subclass. The IgH allele (or the genome) can lack endogenous nucleic acid encoding at least a portion of an IgG2 constant domain, endogenous nucleic acid encoding at least a portion of an IgG3 constant domain, or endogenous nucleic acid encoding at least a portion of an IgG4 constant domain. The IgH allele (or the genome) can lack endogenous nucleic acid encoding at least a portion of an IgG2a constant domain, endogenous nucleic acid encoding at least a portion of an IgG2b constant domain, endogenous nucleic acid encoding at least a portion of an IgG2c constant domain, endogenous nucleic acid encoding at least a portion of an IgG3 constant domain, and endogenous nucleic acid encoding at least a portion of an IgG4 constant domain. The IgH allele (or the genome) can lack endogenous nucleic acid encoding each of the IgG2 constant domains, endogenous nucleic acid encoding each of the IgG3 constant domains, or endogenous nucleic acid encoding each of the IgG4 constant domains. The IgH allele (or the genome) can lack endogenous nucleic acid encoding each of the IgG2a constant domains, endogenous nucleic acid encoding each of the IgG2b constant domains, endogenous nucleic acid encoding each of the IgG2c constant domains, endogenous nucleic acid encoding each of the IgG3 constant domains, or endogenous nucleic acid encoding each of the IgG4 constant domains. The IgH allele (or the genome) can lack endogenous nucleic acid encoding at least a portion of an IgM constant domain, endogenous nucleic acid encoding at least a portion of an IgD constant domain, endogenous nucleic acid encoding at least a portion of an IgE constant domain, and endogenous nucleic acid encoding at least a portion of an IgA constant domain. The IgH allele (or the genome) can lack endogenous nucleic acid encoding each of the IgM constant domains, endogenous nucleic acid encoding each of the IgD constant domains, endogenous nucleic acid encoding each of the IgE constant domains, or endogenous nucleic acid encoding each of the IgA constant domains. The IgH allele (or the genome) can lack endogenous nucleic acid encoding each of the IgM constant domains.

The IgH allele (or the genome) can lack endogenous nucleic acid encoding each of the IgD constant domains. The IgH allele (or the genome) can lack endogenous nucleic acid encoding each of the IgE constant domains. The IgH allele (or the genome) can lack endogenous nucleic acid encoding IgA CH1 and CH2 constant domains. The IgH allele (or the genome) can lack nucleic acid encoding the endogenous CH1 domain. The IgH allele (or the genome) can comprise an endogenous Ep. The first nucleic acid sequence encoding a full length CH2 domain downstream of the endogenous Eμ can be nucleic acid encoding an IgG CH2 domain. The first nucleic acid sequence encoding a full length CH2 domain downstream of the endogenous Ep can be nucleic acid encoding an IgG1 CH2 domain. The IgH allele (or the genome) can comprise an endogenous Sμ, an endogenous Iμ promoter, an endogenous Iμ exon, or a combination thereof. The first nucleic acid sequence encoding a full length CH2 domain downstream of the endogenous Sμ, the endogenous Iμ promoter, or the endogenous Iμ exon can be nucleic acid encoding an IgG CH2 domain. The first nucleic acid sequence encoding a full length CH2 domain downstream of the endogenous Sμ, the endogenous Iμ promoter, or the endogenous Iμ exon can be nucleic acid encoding an IgG1 CH2 domain. The IgH allele (or the genome) can comprise an endogenous 3′γ1E. The IgH allele (or the genome) can lack endogenous nucleic acid encoding a full length CH2 domain downstream of the endogenous 3′γ1E. The IgH allele (or the genome) can comprise an endogenous 5′hsR1. The first nucleic acid sequence encoding a full length CH2 domain upstream of the endogenous 5′hsR1 can be nucleic acid encoding an IgG CH2 domain. The first nucleic acid sequence encoding a full length CH2 domain upstream of the endogenous 5′hsR1 can be nucleic acid encoding an IgG1 CH2 domain. The IgH allele (or the genome) can comprise an endogenous 3′RR. The first nucleic acid sequence encoding a full length CH2 domain upstream of the endogenous 3′RR can be nucleic acid encoding an IgG CH2 domain. The first nucleic acid sequence encoding a full length CH2 domain upstream of the endogenous 3′RR can be nucleic acid encoding an IgG1 CH2 domain. The IgH allele (or the genome) can comprise an endogenous 3′CBE. The first nucleic acid sequence encoding a full length CH2 domain upstream of the endogenous 3′CBE can be nucleic acid encoding an IgG CH2 domain. The first nucleic acid sequence encoding a full length CH2 domain upstream of the endogenous 3′CBE can be nucleic acid encoding an IgG1 CH2 domain. At least one allele of the genome can lack at least a portion of the endogenous Ig heavy chain variable region. At least one allele of the genome can lack all the exons of the endogenous Ig heavy chain variable region. Both alleles of the genome can lack all the exons of the endogenous Ig heavy chain variable region. Neither allele of the genome can comprise an exogenous exon of an Ig heavy chain variable region. The non-human animal can be a non-human animal that does not produce Ig heavy chains. The IgH allele (or the genome) can comprise exogenous nucleic acid encoding one or more human Ig heavy chain variable region gene segments. The IgH allele (or the genome) can comprise one or more exogenous human Ig VH gene segments. The IgH allele (or the genome) can comprise three or more human Ig VH gene segments. The IgH allele (or the genome) can comprise 26 or more human Ig VH gene segments. The IgH allele (or the genome) can comprise 65 or more human Ig VH gene segments. The IgH allele (or the genome) can comprise 126 human Ig VH gene segments. The IgH allele (or the genome) can comprise 13 or more human Ig VD gene segments. The IgH allele (or the genome) can comprise 27 human Ig VD gene segments. The IgH allele (or the genome) can comprise three or more human Ig VJ gene segments. The IgH allele (or the genome) can comprise 9 human Ig VJ gene segments. The genome can comprise 126 human Ig VH gene segments, 27 or more human Ig VD gene segments, and 9 human Ig VJ gene segments. The non-human animal can produce human-non-human chimeric Ig heavy chain antibodies. The variable region domain of the human-non-human chimeric Ig heavy chain antibodies can be fully human. The IgH allele (or the genome) can comprise exogenous nucleic acid encoding one or more human Ig light chain variable region gene segments. The IgH allele (or the genome) can comprise one or more exogenous human Igκ variable gene segments. The IgH allele (or the genome) can comprise 20 or more exogenous human Igκ variable gene segments. The IgH allele (or the genome) can comprise 40 exogenous human Igκ variable gene segments. The IgH allele (or the genome) can comprise one or more exogenous human Igλ variable gene segments. The IgH allele (or the genome) can comprise 10 or more exogenous human Igλ variable gene segments. The IgH allele (or the genome) can comprise 20 exogenous human Igλ variable gene segments. The IgH allele (or the genome) can comprise one or more human Igκ VJ gene segments. The IgH allele (or the genome) can comprise five human Igλ VJ gene segments. The IgH allele (or the genome) can comprise one or more human Ig, VJ gene segments. The IgH allele (or the genome) can comprise four human Igλ VJ gene segments.

The IgH allele (or the genome) can comprise 40 human Igλ variable gene segments and five human Igλ VJ gene segments. The IgH allele (or the genome) can comprise 20 human Igλ variable gene segments and four human Igλ VJ gene segments. The non-human animal can produce human-non-human chimeric Ig heavy chain antibodies. The variable region domain of the human-non-human chimeric Ig heavy chain antibodies can be fully human of light chain origin. The non-human animal can be of a first non-human species, and the IgH allele (or the genome) can comprise exogenous nucleic acid encoding one or more Ig heavy chain variable region gene segments of a second non-human species that is different from the first non-human species. The IgH allele (or the genome) can comprise one or more Ig VH gene segments of the second non-human species. The IgH allele (or the genome) can comprise 10 or more Ig VH gene segments of the second non-human species. The IgH allele (or the genome) can comprise all the Ig VH gene segments of the second non-human species. The IgH allele (or the genome) can comprise three or more Ig VD gene segments of the second non-human species. The IgH allele (or the genome) can comprise all the Ig VD gene segments of the second non-human species. The IgH allele (or the genome) can comprise three or more Ig VJ gene segments of the second non-human species. The IgH allele (or the genome) can comprise all the Ig VJ gene segments of the second non-human species. The IgH allele (or the genome) can comprise all the Ig VH gene segments, Ig VD gene segments, and Ig VJ gene segments of the second non-human species. The non-human animal can produce chimeric heavy chain antibodies of the first and second species. The variable region domain of the chimeric heavy chain antibodies can be fully that of the second species. The first species can be a mouse species. The second species can be a bovine species, a shark species, or an alpaca species. The IgH allele (or the genome) can comprise at least one exogenous recombinase site recognition nucleic acid sequence. The at least one exogenous recombinase site recognition nucleic acid sequence can be located upstream of the endogenous nucleic acid encoding the CH2 or CH3 domain of the IgG subclass. The IgH allele (or the genome) can comprise one, two, three, four, five, six, seven, eight, nine, or ten different exogenous recombinase site recognition nucleic acid sequences. The IgH allele (or the genome) can comprise at least three different exogenous recombinase site recognition nucleic acid sequences. The IgH allele (or the genome) can comprise at least five different exogenous recombinase site recognition nucleic acid sequences. Each of the different exogenous recombinase site recognition nucleic acid sequences is located less than 2.5 Mb upstream of an endogenous Ep. Each of the different exogenous recombinase site recognition nucleic acid sequences is located less than 2.0 Mb, less than 1.5 Mb, less than 1.0 Mb, less than 500 kb, or less than 250 kb upstream of an endogenous Ep. Each of the different exogenous recombinase site recognition nucleic acid sequences is located less than 200 kb, less than 100 kb, less than 50 kb, less than 25 kb, or less than 10 kb upstream of an endogenous Ep. Each of the different exogenous recombinase site recognition nucleic acid sequences can be located less than 500 kb upstream of an endogenous Ep. Each of the different exogenous recombinase site recognition nucleic acid sequences can be located less than 250 kb upstream of an endogenous Ep. Each of the different exogenous recombinase site recognition nucleic acid sequences can be located less than 200 kb upstream of an endogenous Ep.

In another aspect, this document features a DNA comprising a genetically modified non-human immunoglobulin heavy chain (IgH) allele, wherein the genetically modified non-human IgH allele lacks one or more nucleic acid sequences encoding at least a portion of one or more endogenous constant domains comprising a CH1 constant domain of an IgG subclass, an IgM constant domain, an IgD constant domain, an IgE constant domain, an IgA constant domain, or any combination thereof. The DNA can be a germline genomic DNA.

The genetically modified non-human IgH allele can lack one or more nucleic acid sequences encoding at least a portion of one or more endogenous constant domains comprising a CH1 constant domain of an IgG subclass. The IgG subclass can comprise an IgG1, IgG2a, IgG2b, IgG2c, IgG3, or IgG4 subclass. The IgG subclass can be the IgG1 subclass. The genetically modified non-human IgH allele can comprise a nucleic acid sequence (Cγ1-ΔCH1) encoding a CH1-truncated IgG1 constant domain (IgG1ΔCH1). The genetically modified non-human IgH allele can comprise a nucleic acid sequence encoding a hinge (H) domain, a CH2 domain, a CH3 domain of the IgG subclass, or any combination thereof. The genetically modified non-human IgH allele can lack one or more nucleic acid sequences encoding at least a portion of one or more endogenous constant domains comprising an IgG2 constant domain, IgG3 constant domain, IgG4 constant domain, or any combination thereof. The genetically modified non-human IgH allele can comprise one or more endogenous enhancers comprising Ep, 3′γ1E, 5′hsR1, 3′RR, or any combination thereof. The genetically modified non-human IgH allele can comprise an Iμ promoter, an Iμ exon, or both. The genetically modified non-human IgH allele can comprise a switch tandem repeat element (Sμ). IgG1 expression can be driven by the Eμ, Iμ promoter, Sμ, or any combination thereof. The genetically modified non-human IgH allele can lack one or more endogenous switch regions comprising Sγ3, Sγ1, Sγ2b, Sγ2c, Sε, Sα, or any combination thereof. The genetically modified non-human IgH allele can comprise the following components (from 5′ to 3′): Eμ, Iμ promoter, Iμ exon, Sμ, Cγ1-ΔCH1, 3′γ1E, 5′hsR1, and 3′RR. The genetically modified non-human IgH allele can comprise a flippase recognition target (frt) site. The genetically modified non-human IgH allele can comprise endogenous V gene segments, D gene segments, J gene segments, or any combination thereof. The genetically modified non-human IgH allele can lack at least one endogenous V gene segments, D gene segments, J gene segments, or any combination thereof. The genetically modified non-human IgH allele can comprise a docking cassette.

The docking cassette can comprise a left and right homology arm, an frt site, an attB site, a promoter, a loxP site, a nucleic acid sequence encoding a selection marker, or any combination thereof. The docking cassette can comprise a nucleic acid sequence encoding a selection marker. The selection marker can comprise geneticin, hydromycin, puromycin, or any combination thereof. The genetically modified non-human IgH allele can encode an IgG heavy chain antibody. The genetically modified non-human IgH allele can comprise exogenous V gene segments, exogenous D gene segments, exogenous J gene segments, or any combination thereof. The exogenous gene segments can be selected from the group consisting of human, mouse, rat, bovine, alpaca, and shark gene segments. The exogenous gene segments can comprise human gene segments. The genetically modified non-human IgH allele can comprise one or more human VH gene segments, one or more human DH gene segments, and one or more human JH gene segments. The genetically modified non-human IgH allele can comprise at least 10, 20, 30, 40, 50, 60, 80, 100, 120, or 126 human VH gene segments. The genetically modified non-human IgH allele can comprise at least 10, 15, 20, 25, or 27 human DH gene segments. The genetically modified non-human IgH allele can comprise at least 1, 2, 3, 4, 5, 6, 7, 8, or 9 human JH gene segments. The genetically modified non-human IgH allele can comprise 126 human VH gene segments, 27 human DH gene segments, and 9 human JH gene segments. The genetically modified non-human IgH allele can comprise one or more bovine gene segments. The one or more bovine gene segments can comprise an L1 exon, an L2 exon of IGHV1-7, a coding segment of IGHD8-2, a coding sequence of IGHJ2-4, a IGH2-4 splice donor, or any combination thereof. The one or more bovine gene segments can comprise IGHD4-1, IGHD5-3, IGHD8-2, IGHD1-3, IGHD7-3, IGHD7-4, IGHD6-3, IGHD3-3, or any combination thereof. The one or more bovine gene segments can comprise a nucleic acid sequence selected from SEQ ID NOs:42-49 and 57. The DNA can comprise one or more human VH gene segments. The DNA can comprise one or more human J_{H} gene segments. The genetically modified non-human IgH allele can comprise one or more alpaca gene segments. The one or more alpaca gene segments can comprise VHH3-1, VHH3-S1, VHH3-S2, VHH3-S9, VHH3-S10, or any combination thereof. The alpaca gene segments can comprise a nucleic acid sequence selected from SEQ ID NOs:50-54. The DNA can comprise one or more human VH gene segments. The DNA can comprise one or more human JH gene segments. The genetically modified non-human IgH allele can comprise one or more shark gene segments. The one or more shark gene segments can comprise VNAR-L38968, VNAR-L38967, or both. The shark gene segments can comprise a nucleic acid sequence selected from SEQ ID NOs:55-56.

The DNA can comprise one or more human VH gene segments. The DNA can comprise one or more human J_{H} gene segments. The genetically modified non-human IgH allele can encode an IgG heavy chain antibody, and the IgG heavy chain antibody can comprise a kappa light chain variable domain, a lambda light chain variable domain, or both. The genetically modified non-human IgH allele can comprise one or more exogenous human lambda light chain (LV) gene segments. The one or more human LV gene segments can comprise CH17-262M19, CH17-329P5, CH17-238D3, CH17-261A15, CH17-264L24, CH17-117C7, RP11-1040J16, CH17-320F4, or any combination thereof. The genetically modified non-human IgH allele can comprise one or more exogenous human kappa light chain (KV) gene segments. The one or more human KV gene segments can comprise CH17-272M2, CH17-405H5, CH17-140P2, CH17-13E7, CH17-84J8, CH17-53L15, or any combination thereof.

The DNA can comprise one or more human VH gene segments. The DNA can comprise one or more human JH gene segments.

In another aspect, this document features a genetically modified cell comprising a DNA comprising a genetically modified non-human immunoglobulin heavy chain (IgH) allele, wherein the genetically modified non-human IgH allele lacks one or more nucleic acid sequences encoding at least a portion of one or more endogenous constant domains comprising a CH1 constant domain of an IgG subclass, an IgM constant domain, an IgD constant domain, an IgE constant domain, an IgA constant domain, or any combination thereof. The DNA can be a germline genomic DNA. The genetically modified non-human IgH allele can lack one or more nucleic acid sequences encoding at least a portion of one or more endogenous constant domains comprising a CH1 constant domain of an IgG subclass. The IgG subclass can comprise an IgG1, IgG2a, IgG2b, IgG2c, IgG3, or IgG4 subclass. The IgG subclass can be the IgG1 subclass. The genetically modified non-human IgH allele can comprise a nucleic acid sequence (Cγ1-ΔCH1) encoding a CH1-truncated IgG1 constant domain (IgG1ΔCH1). The genetically modified non-human IgH allele can comprise a nucleic acid sequence encoding a hinge (H) domain, a CH2 domain, a CH3 domain of the IgG subclass, or any combination thereof. The genetically modified non-human IgH allele can lack one or more nucleic acid sequences encoding at least a portion of one or more endogenous constant domains comprising an IgG2 constant domain, IgG3 constant domain, IgG4 constant domain, or any combination thereof. The genetically modified non-human IgH allele can comprise one or more endogenous enhancers comprising Eμ, 3′γ1E, 5′hsR1, 3′RR, or any combination thereof. The genetically modified non-human IgH allele can comprise an Iμ promoter, an Iμ exon, or both. The genetically modified non-human IgH allele can comprise a switch tandem repeat element (Sμ). IgG1 expression can be driven by the Eμ, Iμ promoter, Sμ, or any combination thereof. The genetically modified non-human IgH allele can lack one or more endogenous switch regions comprising Sγ3, Sγ1, Sγ2b, Sγ2c, SE, Sa, or any combination thereof. The genetically modified non-human IgH allele can comprise the following components (from 5′ to 3′): Eμ, Iμ promoter, Iμ exon, Sμ, Cγ1-ΔCH1, 3′γ1E, 5′hsR1, and 3′RR. The genetically modified non-human IgH allele can comprise a flippase recognition target (frt) site. The genetically modified non-human IgH allele can comprise endogenous V gene segments, D gene segments, J gene segments, or any combination thereof. The genetically modified non-human IgH allele can lack at least one endogenous V gene segments, D gene segments, J gene segments, or any combination thereof. The genetically modified non-human IgH allele can comprise a docking cassette. The docking cassette can comprise a left and right homology arm, an frt site, an attB site, a promoter, a loxP site, a nucleic acid sequence encoding a selection marker, or any combination thereof. The docking cassette can comprise a nucleic acid sequence encoding a selection marker. The selection marker can comprise geneticin, hydromycin, puromycin, or any combination thereof. The genetically modified non-human IgH allele can encode an IgG heavy chain antibody. The genetically modified non-human IgH allele can comprise exogenous V gene segments, exogenous D gene segments, exogenous J gene segments, or any combination thereof. The exogenous gene segments can be selected from the group consisting of human, mouse, rat, bovine, alpaca, and shark gene segments. The exogenous gene segments can comprise human gene segments. The genetically modified non-human IgH allele can comprise one or more human VH gene segments, one or more human DH gene segments, and one or more human JH gene segments. The genetically modified non-human IgH allele can comprise at least 10, 20, 30, 40, 50, 60, 80, 100, 120, or 126 human VH gene segments. The genetically modified non-human IgH allele can comprise at least 10, 15, 20, 25, or 27 human DH gene segments. The genetically modified non-human IgH allele can comprise at least 1, 2, 3, 4, 5, 6, 7, 8, or 9 human JH gene segments. The genetically modified non-human IgH allele can comprise 126 human VH gene segments, 27 human DH gene segments, and 9 human JH gene segments. The genetically modified non-human IgH allele can comprise one or more bovine gene segments. The one or more bovine gene segments can comprise an L1 exon, an L2 exon of IGHV1-7, a coding segment of IGHD8-2, a coding sequence of IGHJ2-4, a IGH2-4 splice donor, or any combination thereof. The one or more bovine gene segments can comprise IGHD4-1, IGHD5-3, IGHD8-2, IGHD1-3, IGHD7-3, IGHD7-4, IGHD6-3, IGHD3-3, or any combination thereof. The one or more bovine gene segments can comprise a nucleic acid sequence selected from SEQ ID NOs:42-49 and 57. The DNA can comprise one or more human VH gene segments. The DNA can comprise one or more human JH gene segments. The genetically modified non-human IgH allele can comprise one or more alpaca gene segments. The one or more alpaca gene segments can comprise VHH3-1, VHH3-S1, VHH3-S2, VHH3-S9, VHH3-S10, or any combination thereof. The alpaca gene segments can comprise a nucleic acid sequence selected from SEQ ID NOs:50-54. The DNA can comprise one or more human VH gene segments. The DNA can comprise one or more human JH gene segments. The genetically modified non-human IgH allele can comprise one or more shark gene segments. The one or more shark gene segments can comprise VNAR-L38968, VNAR-L38967, or both. The shark gene segments can comprise a nucleic acid sequence selected from SEQ ID NOs:55-56. The DNA can comprise one or more human VH gene segments. The DNA can comprise one or more human JH gene segments. The genetically modified non-human IgH allele can encode an IgG heavy chain antibody, and the IgG heavy chain antibody can comprise a kappa light chain variable domain, a lambda light chain variable domain, or both. The genetically modified non-human IgH allele can comprise one or more exogenous human lambda light chain (LV) gene segments. The one or more human LV gene segments can comprise CH17-262M19, CH17-329P5, CH17-238D3, CH17-261A15, CH17-264L24, CH17-117C7, RP11-1040J16, CH17-320F4, or any combination thereof. The genetically modified non-human IgH allele can comprise one or more exogenous human kappa light chain (KV) gene segments. The one or more human KV gene segments can comprise CH17-272M2, CH17-405H5, CH17-140P2, CH17-13E7, CH17-84J8, CH17-53L15, or any combination thereof. The DNA can comprise one or more human VH gene segments. The DNA can comprise one or more human JH gene segments. The cell can be a non-human animal cell. The cell can be a mammalian cell. The mammalian cell can be a mouse, rat, bovine, alpaca, cat, dog, rabbit, pig, monkey, or chimpanzee cell. The cell can be a mouse cell. The cell can be a shark cell. The cell can be a human cell. The cell can be a stem cell. The stem cell can be an embryonic stem cell (ESC) or induced pluripotent stem cells (iPSCs). The cell can be a B cell.

In another aspect, this document features a genetically modified non-human animal, wherein the genetically modified non-human animal comprises a cell of the preceding paragraph. The non-human animal can be mammal. The mammal can be a mouse, rat, bovine, alpaca, cat, dog, rabbit, pig, monkey, or chimpanzee. The non-human animal can be a mouse. The genetically modified non-human animal can comprise a cell expressing an IgG heavy chain antibody. The IgG heavy chain antibody can be secreted into the serum of the genetically modified non-human animal. The IgG heavy chain antibody can be a CH1-truncated IgG1 heavy chain antibody (IgG1ΔCH1). The IgG heavy chain antibody can lack a light chain. The IgG heavy chain antibody can comprise a hinge domain, CH2 domain, a CH3 domain, or any combination thereof. The cell expressing the IgG heavy chain antibody can be a cell that does not express an IgM antibody, an IgD antibody, an IgE antibody, an IgG3 antibody, an IgG2b antibody, an IgG2c antibody, an IgA antibody, or any combination thereof. The IgG heavy chain antibody can be a human IgG heavy chain antibody. The IgG heavy chain antibody can comprise an exogenous variable domain selected from the group consisting of human, mouse, rat, bovine, alpaca, and shark variable domains. The IgG heavy chain antibody can comprise a kappa light chain variable domain, a lambda light chain variable domain, or both.

In another aspect, this document features a method for preparing a genetically modified non-human animal. The method comprises (a) deleting one or more nucleic acid sequences from a non-human immunoglobulin heavy chain (IgH) allele, wherein the deleted one or more nucleic acid sequences encode at least a portion of one or more endogenous constant domains comprising a CH1 constant domain of an IgG subclass, an IgM constant domain, an IgD constant domain, an IgE constant domain, an IgA constant domain, or any combination thereof, thereby generating a genetically modified non-human IgH allele in a germline genomic DNA; (b) implanting a cell comprising the germline genomic DNA into a blastocyst; (c) implanting the blastocyst into a pseudo-pregnant non-human animal to obtain a chimeric non-human animal; (d) crossing the chimeric non-human animal to a wild-type non-human animal to produce offspring; (e) screening the offspring for heterozygosity; and (f) identifying the genetically modified non-human animal carrying the deletion of the one or more nucleic acid sequences and capable of producing a heavy chain antibody. The genetically modified non-human animal can be the genetically modified non-human animal of the preceding paragraph. Deleting the one or more nucleic acid sequences can comprise using a CRISPR/Cas genome editing system. The CRISPR/Cas genome editing system can comprise at least one guide RNA (gRNA) targeting an endogenous heavy-chain C region gene and a Cas protein. The Cas protein can comprise a Cas9 protein. The deleted one or more nucleic acid sequences can encode the CH1 constant domain of IgG1, the IgG3 constant domain, the IgM constant domain, and the IgD constant domain. The deleted one or more nucleic acid sequences can encode the IgG2 constant domain and the IgA constant domain.

Deleting the nucleic acid sequence can comprise removing a selection marker from the non-human IgH allele using transient expression of Flp recombinase. The deleted one or more nucleic acid sequences can encode the CH1 constant domain of the IgG subclass, the IgM constant domain, the IgD constant domain, the IgE constant domain, and the IgA constant domain. The method can comprise deleting a nucleic acid sequence from the non-human IgH allele, wherein the nucleic acid sequence comprises endogenous V gene segments, D gene segments, J gene segments or any combination thereof. The method can comprise inserting a docking cassette. The method can comprise contacting the docking cassette with a bacterial artificial chromosome (BAC), wherein the BAC comprises a nucleic acid sequence comprising exogenous V_{H}, D_{H}, and J_{H} gene segments. The method can comprise inserting the exogenous gene segments into the docketing cassette. The exogenous gene segments can be human gene segments.

In another aspect, this document features a genetically modified non-human animal, wherein the genetically modified non-human animal was prepared using the method of the preceding paragraph.

In another aspect, this document features a method for preparing a germline genomic DNA, wherein the method comprises deleting one or more nucleic acid sequences from a non-human immunoglobulin heavy chain (IgH) allele, wherein the deleted one or more nucleic acid sequences encode at least a portion of one or more endogenous constant domains comprising a CH1 constant domain of an IgG subclass, an IgM constant domain, an IgD constant domain, an IgE constant domain, an IgA constant domain, or any combination thereof, thereby generating a genetically modified non-human IgH allele in the germline genomic DNA. The germline genomic DNA can comprise a DNA comprising the genetically modified non-human IgH allele. The genetically modified non-human IgH allele can lack one or more nucleic acid sequences encoding at least a portion of one or more endogenous constant domains comprising a CH1 constant domain of an IgG subclass. The IgG subclass can comprise an IgG1, IgG2a, IgG2b, IgG2c, IgG3, or IgG4 subclass. The IgG subclass can be the IgG1 subclass. The genetically modified non-human IgH allele can comprise a nucleic acid sequence (Cγ1-ΔCH1) encoding a CH1-truncated IgG1 constant domain (IgG1ΔCH1). The genetically modified non-human IgH allele can comprise a nucleic acid sequence encoding a hinge (H) domain, a CH2 domain, a CH3 domain of the IgG subclass, or any combination thereof. The genetically modified non-human IgH allele can lack one or more nucleic acid sequences encoding at least a portion of one or more endogenous constant domains comprising an IgG2 constant domain, IgG3 constant domain, IgG4 constant domain, or any combination thereof. The genetically modified non-human IgH allele can comprise one or more endogenous enhancers comprising Eμ, 3′γ1E, 5′hsR1, 3′RR, or any combination thereof. The genetically modified non-human IgH allele can comprise an Iμ promoter, an Iμ exon, or both. The genetically modified non-human IgH allele can comprise a switch tandem repeat element (Sμ). IgG1 expression can be driven by the Eμ, Iμ promoter, Sμ, or any combination thereof. The genetically modified non-human IgH allele can lack one or more endogenous switch regions comprising Sγ3, Sγ1, Sγ2b, Sγ2c, Sε, Sa, or any combination thereof. The genetically modified non-human IgH allele can comprise the following components (from 5′ to 3′): Eμ, Iμ promoter, Iμ exon, Sμ, Cγ1-ΔCH1, 3′γ1E, 5′hsR1, and 3′RR. The genetically modified non-human IgH allele can comprise a flippase recognition target (frt) site. The genetically modified non-human IgH allele can comprise endogenous V gene segments, D gene segments, J gene segments, or any combination thereof. The genetically modified non-human IgH allele can lack at least one endogenous V gene segments, D gene segments, J gene segments, or any combination thereof. The genetically modified non-human IgH allele can comprise a docking cassette. The docking cassette can comprise a left and right homology arm, an frt site, an attB site, a promoter, a loxP site, a nucleic acid sequence encoding a selection marker, or any combination thereof. The docking cassette can comprise a nucleic acid sequence encoding a selection marker. The selection marker can comprise geneticin, hydromycin, puromycin, or any combination thereof. The genetically modified non-human IgH allele can encode an IgG heavy chain antibody. The genetically modified non-human IgH allele can comprise exogenous V gene segments, exogenous D gene segments, exogenous J gene segments, or any combination thereof. The exogenous gene segments can be selected from the group consisting of human, mouse, rat, bovine, alpaca, and shark gene segments. The exogenous gene segments can comprise human gene segments. The genetically modified non-human IgH allele can comprise one or more human VH gene segments, one or more human DH gene segments, and one or more human JH gene segments. The genetically modified non-human IgH allele can comprise at least 10, 20, 30, 40, 50, 60, 80, 100, 120, or 126 human VH gene segments. The genetically modified non-human IgH allele can comprise at least 10, 15, 20, 25, or 27 human DH gene segments. The genetically modified non-human IgH allele can comprise at least 1, 2, 3, 4, 5, 6, 7, 8, or 9 human JH gene segments. The genetically modified non-human IgH allele can comprise 126 human VH gene segments, 27 human DH gene segments, and 9 human JH gene segments. The genetically modified non-human IgH allele can comprise one or more bovine gene segments. The one or more bovine gene segments can comprise an L1 exon, an L2 exon of IGHV1-7, a coding segment of IGHD8-2, a coding sequence of IGHJ2-4, a IGH2-4 splice donor, or any combination thereof. The one or more bovine gene segments can comprise IGHD4-1, IGHD5-3, IGHD8-2, IGHD1-3, IGHD7-3, IGHD7-4, IGHD6-3, IGHD3-3, or any combination thereof. The one or more bovine gene segments can comprise a nucleic acid sequence selected from SEQ ID NOs:42-49 and 57. The DNA can comprise one or more human VH gene segments. The DNA can comprise one or more human JH gene segments. The genetically modified non-human IgH allele can comprise one or more alpaca gene segments. The one or more alpaca gene segments can comprise VHH3-1, VHH3-S1, VHH3-S2, VHH3-S9, VHH3-S10, or any combination thereof. The alpaca gene segments can comprise a nucleic acid sequence selected from SEQ ID NOs:50-54. The DNA can comprise one or more human VH gene segments. The DNA can comprise one or more human JH gene segments. The genetically modified non-human IgH allele can comprise one or more shark gene segments. The one or more shark gene segments can comprise VNAR-L38968, VNAR-L38967, or both. The shark gene segments can comprise a nucleic acid sequence selected from SEQ ID NOs:55-56. The DNA can comprise one or more human VH gene segments. The DNA can comprise one or more human JH gene segments. The genetically modified non-human IgH allele can encode an IgG heavy chain antibody, and the IgG heavy chain antibody can comprise a kappa light chain variable domain, a lambda light chain variable domain, or both. The genetically modified non-human IgH allele can comprise one or more exogenous human lambda light chain (LV) gene segments. The one or more human LV gene segments can comprise CH17-262M19, CH17-329P5, CH17-238D3, CH17-261A15, CH17-264L24, CH17-117C7, RP11-1040J16, CH17-320F4, or any combination thereof. The genetically modified non-human IgH allele can comprise one or more exogenous human kappa light chain (KV) gene segments. The one or more human KV gene segments can comprise CH17-272M2, CH17-405H5, CH17-140P2, CH17-13E7, CH17-84J8, CH17-53L15, or any combination thereof. The DNA can comprise one or more human VH gene segments. The DNA can comprise one or more human JH gene segments. The IgG constant domain can comprise a constant domain of an IgG subclass. The IgG subclass can comprise an IgG1, IgG2a, IgG2b, IgG2c, IgG3, or IgG4 subclass.

In another aspect, this document features a method of producing an IgG heavy-chain antibody in a genetically modified non-human animal. The method comprises (a) administering an antigen to the genetically modified non-human animal of any of the preceding paragraphs; (b) isolating one or more B cells from the genetically modified non-human animal; (c) isolating mRNA from the one or more B cells; and (d) producing the IgG heavy-chain antibody. The genetically modified non-human animal can comprise a DNA comprising a genetically modified non-human immunoglobulin heavy chain (IgH) allele, wherein the genetically modified non-human IgH allele lacks one or more nucleic acid sequences encoding at least a portion of one or more endogenous constant domains comprising a CH1 constant domain of an IgG subclass, an IgM constant domain, an IgD constant domain, an IgE constant domain, an IgA constant domain, or any combination thereof. The DNA can be a germline genomic DNA. The genetically modified non-human IgH allele can lack one or more nucleic acid sequences encoding at least a portion of one or more endogenous constant domains comprising a CH1 constant domain of an IgG subclass. The IgG subclass can comprise an IgG1, IgG2a, IgG2b, IgG2c, IgG3, or IgG4 subclass. The IgG subclass can be the IgG1 subclass. The genetically modified non-human IgH allele can comprise a nucleic acid sequence (Cγ1-ΔCH1) encoding a CH1-truncated IgG1 constant domain (IgG1ΔCH1). The genetically modified non-human IgH allele can comprise a nucleic acid sequence encoding a hinge (H) domain, a CH2 domain, a CH3 domain of the IgG subclass, or any combination thereof. The genetically modified non-human IgH allele can lack one or more nucleic acid sequences encoding at least a portion of one or more endogenous constant domains comprising an IgG2 constant domain, IgG3 constant domain, IgG4 constant domain, or any combination thereof. The genetically modified non-human IgH allele can comprise one or more endogenous enhancers comprising Eμ, 3′γ1E, 5′hsR1, 3′RR, or any combination thereof. The genetically modified non-human IgH allele can comprise an Iμ promoter, an Iμ exon, or both. The genetically modified non-human IgH allele can comprise a switch tandem repeat element (Sμ). IgG1 expression can be driven by the Eμ, Iμ promoter, Sμ, or any combination thereof. The genetically modified non-human IgH allele can lack one or more endogenous switch regions comprising Sγ3, Sγ1, Sγ2b, Sγ2c, SE, Sa, or any combination thereof. The genetically modified non-human IgH allele can comprise the following components (from 5′ to 3′): Eμ, Iμ promoter, Iμ exon, Sμ, Cγ1-ΔCH1, 3′γ1E, 5′hsR1, and 3′RR. The genetically modified non-human IgH allele can comprise a flippase recognition target (frt) site. The genetically modified non-human IgH allele can comprise endogenous V gene segments, D gene segments, J gene segments, or any combination thereof. The genetically modified non-human IgH allele can lack at least one endogenous V gene segments, D gene segments, J gene segments, or any combination thereof. The genetically modified non-human IgH allele can comprise a docking cassette. The docking cassette can comprise a left and right homology arm, an frt site, an attB site, a promoter, a loxP site, a nucleic acid sequence encoding a selection marker, or any combination thereof. The docking cassette can comprise a nucleic acid sequence encoding a selection marker. The selection marker can comprise geneticin, hydromycin, puromycin, or any combination thereof. The genetically modified non-human IgH allele can encode an IgG heavy chain antibody. The genetically modified non-human IgH allele can comprise exogenous V gene segments, exogenous D gene segments, exogenous J gene segments, or any combination thereof. The exogenous gene segments can be selected from the group consisting of human, mouse, rat, bovine, alpaca, and shark gene segments. The exogenous gene segments can comprise human gene segments. The genetically modified non-human IgH allele can comprise one or more human VH gene segments, one or more human DH gene segments, and one or more human JH gene segments. The genetically modified non-human IgH allele can comprise at least 10, 20, 30, 40, 50, 60, 80, 100, 120, or 126 human VH gene segments. The genetically modified non-human IgH allele can comprise at least 10, 15, 20, 25, or 27 human DH gene segments. The genetically modified non-human IgH allele can comprise at least 1, 2, 3, 4, 5, 6, 7, 8, or 9 human JH gene segments. The genetically modified non-human IgH allele can comprise 126 human VH gene segments, 27 human DH gene segments, and 9 human JH gene segments. The genetically modified non-human IgH allele can comprise one or more bovine gene segments. The one or more bovine gene segments can comprise an L1 exon, an L2 exon of IGHV1-7, a coding segment of IGHD8-2, a coding sequence of IGHJ2-4, a IGH2-4 splice donor, or any combination thereof. The one or more bovine gene segments can comprise IGHD4-1, IGHD5-3, IGHD8-2, IGHD1-3, IGHD7-3, IGHD7-4, IGHD6-3, IGHD3-3, or any combination thereof. The one or more bovine gene segments can comprise a nucleic acid sequence selected from SEQ ID NOs:42-49 and 57. The DNA can comprise one or more human VH gene segments. The DNA can comprise one or more human JH gene segments. The genetically modified non-human IgH allele can comprise one or more alpaca gene segments. The one or more alpaca gene segments can comprise VHH3-1, VHH3-S1, VHH3-S2, VHH3-S9, VHH3-S10, or any combination thereof. The alpaca gene segments can comprise a nucleic acid sequence selected from SEQ ID NOs:50-54. The DNA can comprise one or more human VH gene segments. The DNA can comprise one or more human JH gene segments. The genetically modified non-human IgH allele can comprise one or more shark gene segments. The one or more shark gene segments can comprise VNAR-L38968, VNAR-L38967, or both. The shark gene segments can comprise a nucleic acid sequence selected from SEQ ID NOs:55-56. The DNA can comprise one or more human VH gene segments. The DNA can comprise one or more human JH gene segments. The genetically modified non-human IgH allele can encode an IgG heavy chain antibody, and the IgG heavy chain antibody can comprise a kappa light chain variable domain, a lambda light chain variable domain, or both. The genetically modified non-human IgH allele can comprise one or more exogenous human lambda light chain (LV) gene segments. The one or more human LV gene segments can comprise CH17-262M19, CH17-329P5, CH17-238D3, CH17-261A15, CH17-264L24, CH17-117C7, RP11-1040J16, CH17-320F4, or any combination thereof. The genetically modified non-human IgH allele can comprise one or more exogenous human kappa light chain (KV) gene segments. The one or more human KV gene segments can comprise CH17-272M2, CH17-405H5, CH17-140P2, CH17-13E7, CH17-84J8, CH17-53L15, or any combination thereof. The DNA can comprise one or more human VH gene segments. The DNA can comprise one or more human JH gene segments. The method can comprise sequencing the mRNA isolated from the one or more B cells. The method can comprise identifying a clonal type based on the mRNA sequence. The method can comprise performing a phylogenetic analysis of the clonal type. The IgG heavy-chain antibody can be a humanized IgG heavy-chain antibody. The IgG heavy-chain antibody can be a IgG heavy-chain antibody comprising a human variable region and a non-human constant region.

In another aspect, this document features an IgG heavy chain antibody, wherein the IgG heavy chain antibody is produced by the method of the preceding paragraph.

In another aspect, this document features a recombinant vector system comprising at least one nucleic acid construct encoding a CRISPR/Cas genome editing system comprising a Cas protein and at least one guide RNA (gRNA), wherein the Cas protein and at least one gRNA form a complex that deletes one or more nucleic acid sequences from a non-human immunoglobulin heavy chain (IgH) allele, wherein the deleted one or more nucleic acid sequences encode at least a portion of one or more endogenous constant domains comprising a CH1 constant domain of an IgG subclass, an IgM constant domain, an IgD constant domain, an IgE constant domain, an IgA constant domain, or any combination thereof.

In another aspect, this document features an antibody comprising a variable region comprising (a) SEQ ID NO:4, SEQ ID NO: 10, and SEQ ID NO: 19, or (b) SEQ ID NO:5, SEQ ID NO:11, and SEQ ID NO:20. The antibody can bind to a SARS-CoV2 spike polypeptide. The antibody can be a heavy chain antibody. The antibody can be a singly domain antibody.

In another aspect, this document features an antibody comprising a variable region comprising (a) SEQ ID NO:4, SEQ ID NO:10, and SEQ ID NO:19, expect that SEQ ID NO:19 lacks the first C residue and the last W residue, or (b) SEQ ID NO:5, SEQ ID NO:11, and SEQ ID NO:20, expect that SEQ ID NO:20 lacks the first C residue and the last W residue. The antibody can bind to a SARS-CoV2 spike polypeptide. The antibody can be a heavy chain antibody. The antibody can be a singly domain antibody.

Unless otherwise defined, all technical and scientific terms used herein have the same meaning as commonly understood by one of ordinary skill in the art to which this disclosure pertains. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-1D illustrate production of heavy chain only antibodies from Singularity Musculus mice. (FIG. 1A) The genomic structure of the Igh locus of a wild-type mouse. Mouse V_{H}, D_{H}, and J_{H}, and CH genes are indicated with dark or light boxes, along with the intronic enhancer Ep and super-enhancer 3′RR in ovals. (FIG. 1B) The engineered Singularity Musculus (SM) allele where all other CH genes as well as the CH1 exon of IgG1 are deleted. (FIG. 1C) Tetrameric mouse IgG1 produced from the WT allele. (FIG. 1D) CH1-truncated heavy chain only IgG1 are produced from the Singularity Musculus allele, from which nanobodies can be derived.

FIG. 2 illustrates an exemplary genomic locus of mouse Igh. Shown is a schematic map of the ˜220kb CH region containing the indicated regulatory elements.

FIG. 3A-3E illustrate generation of the Singularity Musculus allele. FIG. 3A shows the mouse wild type Igh locus. FIG. 3B shows a constant region of mouse Igh locus. The region to be deleted in the first round of engineering is indicated in dashed box FIG. 3C shows deletion of Ighm-Ighg1 CH1 exon via CRISPR mediated NHEJ. The region to be deleted in the second round of engineering is indicated in dashed box. FIG. 3D shows deletion of Ighg2b-Igha exons 1-3 via CRISPR mediate HDR. FIG. 3E shows removal of selection cassette by expression of Flp recombinase.

FIG. 4 illustrates exemplary genomic structures of wild-type mouse Igh allele and engineered Singularity Musculus and Singularity HyperDock alleles.

FIGS. 5A-5D illustrate the generation of the Singularity HyperDock allele. FIG. 5A shows a Singularity Musculus allele. (FIG. 5B) The Singularity HyperDock allele is generated by deleting all mouse V_{H}, D_{H}, J_{H} genes (2.58 Mb) and inserting a docking cassette for sequential RMCE via CRISPR mediated HDR. FIG. 5C shows Synteny validation of the Singularity HyperDock allele via expression of Flp recombinase. FIG. 5D shows removal of the selection marker via expression of ΦC31 recombinase.

FIGS. 6A-6B illustrate production of human-mouse chimeric heavy chain only antibodies from exemplary Singularity Sapiens mice. FIG. 6A is a schematic of an exemplary human VH-mouse IgG1-ΔCH1 chimeric antibody that can be used to generate a human VH nanobody. FIG. 6B shows exemplary versions of Singularity Sapiens mice produced by sequential introduction of human V, D, J genes onto the Singularity HyperDock allele. Human VH-CH1-truncated heavy chain only IgG1 will be produced from the Singularity Sapiens mice, from which human VH nanobodies can be derived.

FIGS. 7A-7D illustrate the generation of the Singularity Sapiens allelic series (SSV1-3). The Singularity Sapiens allelic series is generated by inserting engineered human IGH BAC1-3 via sequential RMCE using a list of heterospecific lox sites to swap alternating selection cassettes (neo and hyg) upon expression of Cre recombinase.

FIGS. 8A-8D are schematics of a Singularity Sapiens allelic series (SSV4-5) showing sequential integration of human IGH-BAC4 and IGH-BACS via RMCE into a clone containing human IGH-BAC1, human IGH-BAC2, and human IGH-BAC3, followed by removal of the selection marker cassette with via expression of ΦC31 recombinase.

FIG. 9 illustrates human IGH BACs based on human genome GRCh38/hg38 assembly GENCODE Genes Track (Version 36, Oct 2020) of the IGH gene locus showing the human gene segments for variable heavy (IGHV), diversity heavy (IGHD), joining heavy (IGHJ), and constant heavy IGHM and IGHD. Five BAC constructs (hIGH BAC1-5, boundaries marked in dashed boxes) carrying human IGHV, IGHD, and IGHJgene segments were engineered with the corresponding source BACs (solid boxes) via recombineering. The engineered BACs were then used to reconstruct the entire human V-D-J genomic region into the Singularity HyperDock allele via RMCE as described herein. The numbers of V, D, and J gene segments included in each engineered BAC construct are indicated.

FIG. 10 illustrates an example of BAC recombineering. Source BACs are modified by bacterial homologous recombination (recombineering) to incorporate the appropriate selectable markers and recombination sites at the desired positions. Shown is an engineering process of hIgH-BAC1.

FIG. 11 illustrates VH exon validation, showing a PCR-based validation of Singularity Sapiens (SSV4) containing 37 functional human VH exons integrated at the IGH locus. PCR results were run on a Qiagen Qiaxel DNA High resolution cartridge. Top and bottom bands denoted Qiagen QX alignment marker 15 bp/3 kb (Cat #929522) that was run alongside Qiagen QX size marker 100 bp-2.5 kb (Cat #929559). The PCR products were verified by sanger sequencing to match the corresponding VH genes.

FIG. 12 illustrates an exemplary method of complex BAC recombineering. Source BACs are sequentially modified by bacterial homologous recombination (recombineering) to incorporate the appropriate selectable markers and recombination sites at the desired positions. An example is shown for the engineering process for hIGH-BACS from three sourced BACs.

FIGS. 13A-13B illustrate engineering of a mutant mouse lacking the Kappa light chain. FIG. 13A is a schematic showing deletion of the mouse IG Kappa and insertion of a docking site via CRISPR mediated HDR. FIG. 13B is genotyping PCR results confirming IGK HyperDock/KO allele in F1 mice.

FIGS. 14A-14B illustrate engineering of a mutant mouse lacking the Lambda light chain. FIG. 14A is a schematic showing deletion of the entire mouse IG Lambda locus via CRISPR mediated NHEJ. FIG. 14B is a PCR result confirming the generation of the IGL KO allele in ES cells.

FIGS. 15A-15D illustrate that Singularity Musculus mice produce HcAbs of CH1 truncated IgG1 only. Schematics of the Igh locus in WT (FIG. 15A) and SM (FIG. 15B) mice. Validation of Singularity Musculus mice is shown by RT-PCR (spleens) (FIG. 15C) and Western blots (plasma) (FIG. 15D).

FIGS. 16A-16D illustrate that Singularity Sapiens mice produce human-mouse chimeric heavy chain IgG1s. FIG. 16A shows a Singularity Musculus allele. FIG. 16B shows a Singularity Sapiens V1 allele, which contains all human J_{H}, all human D_{H}, and 3 human VH genes. (FIG. 16C) RT-PCR shows the specific expression of human-mouse chimeric IgG1ΔCH1 transcripts in Singularity Sapiens V1 mice. FIG. 16D shows sequencing validated the production of human-mouse chimeric transcripts (SEQ ID NO:36).

FIGS. 17A-17B. FIG. 17A is a schematic of an exemplary human VH-mouse IgG1-ΔCH1 chimeric antibody that can be used to generate a human VH nanobody. FIG. 17B shows Western blot of IgM and IgG1 in immunized WT and Singularity Sapiens mice (SSV1).

FIGS. 18A-18B illustrate spleen morphology and IgM and IgG expression in B cells of in Singularity Musculus mice. FIG. 18A shows spleens of wild type and Singularity Musculus mice. FIG. 18B shows a flow cytometry analysis of splenocytes demonstrating the absence of IgM but normal IgG expression in CD19 positive B cells.

FIG. 19A-19B illustrate B cell markers in Singularity Sapiens mice. FIG. 19A shows a flow cytometry analysis demonstrating the presence of IgM⁺ IgD⁺ B cells in wildtype mice but their absence in Singularity Sapiens mice (SSV2). FIG. 19B shows a flow cytometry analysis demonstrating the differential abundance of IgG1+B cells in wildtype mice and in Singularity Sapiens mice (SSV2).

FIGS. 20A-20B illustrate that Singularity Musculus mice mount robust humoral immune responses upon antigen challenge. FIG. 20A shows ELISA results of plasma samples from pre-bleed wild-type and Singularity Musculus animals. FIG. 20B shows ELISA results of plasma samples from Day 28 terminal bleed of the same animals immunized with SARS-CoV2 Spike Active Trimer protein (SAT) as compared to a commercial control antibody against SARS-CoV2 Spike protein S1 subunit (S1 mAb Control).

FIG. 21A-21B illustrate that Singularity Musculus mice and Singularity Sapiens mice mount robust humoral immune responses upon a variety of antigen challenges. FIG. 21A shows ELISA results of plasma samples from Day 51 terminal bleed wildtype (WT), Singularity Musculus (SM), and Singularity Sapiens (SSV1) animals upon antigen challenge to SAT. FIG. 21B shows ELISA results of plasma samples from Day 51 terminal bleed animals immunized with human PD-L1 as compared to a commercial human PD-L1 antibody.

FIG. 22 is a schematic illustrating IgG1 transcripts of WT and Singularity Musculus mice and primer locations for 5′RACE amplification for next generation sequencing analysis.

FIGS. 23A-23C illustrate that Singularity Musculus mice exhibit comparable antibody diversity as in the wild-type mice. VH diversity (FIG. 23A); J_{H} diversity (FIG. 23B); and CDR3 length diversity (FIG. 23C) of all clonotypes identified from two wild type and two Singularity Musculus mice immunized with SAT are shown.

FIGS. 24A-24C illustrate IGHV diversity of clonotypes identified in WT and SM mice. FIGS. 24A and B shows IGHV usage in SM mice immunized with the indicated antigens. (FIG. 24C) More IGHV segments are accessible to SM mice than WT mice.

FIGS. 25A-25B illustrate IGHJ utilization in WT and SM mice. FIG. 25A shows IGHJ usage in SM mice immunized with the indicated antigens. (FIG. 25B) Different usage of certain IGHJ segments in SM mice compared to WT mice was observed.

FIGS. 26A-26B illustrate CDR3 length distribution in WT and SM mice. FIG. 26A shows the distribution of CDR3 length among clonotypes in SM and WT mice in response to the indicated antigen. FIG. 26B shows the average CDR3 length observed in SM and WT mice.

FIG. 27 illustrates somatic hypermutation in the Singularity Musculus mice. The histograms show the number of amino acid changes at each position in the heavy-chain variable region, as compared to the corresponding germline sequence, for the top 100 most abundant nanobody clonotypes identified from one naive and three Singularity Musculus mice immunized with SAT. VH residue position numbering is based on the IMGT scheme. Most significant changes occurred in the CDR regions.

FIG. 28 illustrates a flow chart for an exemplary NGS-guided, single cell-independent nanobody discovery process.

FIG. 29 illustrates a phylogenetic tree of selected clonotypes identified by next generation sequencing of HcAb repertoire of Singularity Musculus mice immunized with SAT. Top ranked (based on abundance) clonotypes were selected for each immunized animal for high throughput synthesis, cloning, expression, and ELISA screening for antigen affinity followed by competitive ELISA for inhibitor (neutralizing) activity for spike-ACE2 receptor binding. Antigen-specific clones are indicated in grey shading, and neutralizing clones are indicated with an asterisk.

FIGS. 30A-30B illustrate vector used for expressing nanobodies. FIG. 30A shows the plasmid map of the pFUSE-hIgG1-Fc2 expression vector and the restriction sites (EcoRI and NcoI) for inserting VH sequence. FIG. 30B shows an exemplary Nb-human Fc fusion that can be generated from the pFUSE-hIgG1-Fc2 expression vector.

FIG. 31 illustrates ELISA screens for binders in immunized WT and SM mice. The numbers of clonotypes screened and binders identified from WT and SM mice after immunization with indicated antigens are provided. Binding results (ELISA results OD₄₅₀) for each clonotype are provided.

FIG. 32 contains pie charts from data in FIG. 31 showing the proportion of binders having the indicated binding affinity obtained from WT and SM mice. Each chart shows the proportion of binders as determined by ELISA of nanobody binding to the indicated antigen.

FIGS. 33A-33B illustrate exemplary antibody structures under unreduced and reduced conditions of WT IgG1s and Nb-human Fc fusions (FIG. 33A), and confirmation of size reduction with SDS-PAGE gels of a S1 mAb control (WT tetrameric IgG1) and purified SAT nanobody-Fc fusions (heavy chain only IgG1) (FIG. 33B). The expressed Nb-Fc human fusions are homodimers.

FIGS. 34A-34B illustrate SDS-PAGE gels of purified SAT human nanobody-human Fc fusions (heavy chain only IgG1). FIG. 34A shows gel run under non-reduced conditions.

FIG. 34B shows gel run under reduced conditions. The expressed human Nb-human Fc fusions are observed as homodimers.

FIG. 35 illustrates size exclusion chromatography of two human nanobody-human Fc fusion proteins. Purified human Nb-human Fc fusion proteins against SAT antigen were run through a size exclusion column to assess protein aggregation.

FIGS. 36A-36B illustrate characterization of purified SAT Nb-human Fc fusions for antigen binding affinity and SARS-CoV2 neutralization potency against a RBD Nb-Fc control (HAb8-S). FIG. 36A shows EC₅₀ values for binding affinity. FIG. 36B shows IC₅₀ values for neutralization potency.

FIGS. 37A-37B illustrate phylogenetic relations (FIG. 37A) and somatic hypermutation analysis (FIG. 37B) of closely related VH sequences identified using two SARS-CoV2 neutralizing nanobodies (indicated with asterisk). Closely related, low abundance clonotypes were identified for secondary screening for high affinity and high potency nanobodies. The sequences of the nanobody clones in FIG. 37B from top to bottom are set forth in SEQ ID NOs:25-35, respectively.

FIG. 38 is a table presenting binding kinetics of mouse and human SAT nanobody-human Fc fusion molecules. Binding of mouse and human Nb-human Fc fusion proteins to the recombinant SAT protein was assayed by Bio-Layer Interferometry (BLI) using the Octet. These results demonstrate that the engineered mice provided herein can be used to obtain high affinity mouse nanobodies and high affinity human nanobodies.

FIG. 39 illustrates binding kinetics of exemplary human nanobody-human Fc fusion molecules. Sensograms obtained by BLI of purified human Nb-human Fc fusion proteins in the presence of recombinant SAT.

FIG. 40 illustrates melting peaks of human nanobody-human Fc fusion molecules. Melting curves of purified human SAT Nb-human Fc fusion proteins were generated via pFUSE-hIgG1-Fc2 expression vector in Expi293F cells. These results demonstrate that the human Nb-human Fc molecules can exhibit thermos-stability similar to that of known natural nanobodies.

FIGS. 41A-41B illustrate cell binding assay results of mouse nanobody-human Fc fusion molecules and human nanobody-human Fc fusion molecules. FIG. 41A is an exemplary result showing positive control and negative control of HEK293 expressing SARS-Cov2 Spike proteins (top panel) or HEK293 (bottom panel) incubated in the presence of purified mouse or human Nb-human Fc fusion proteins. Cell binding was assessed using fluorescent secondary antibody against the Fc region of the Nb-Fc molecule. FIG. 41B shows summary results of the cell binding data for mouse and human Nb-human Fc fusion proteins.

FIG. 42 contains graphs showing the cell binding results for all Nb-human Fc fusions of FIG. 41A-41B. Top panel, mouse Nb-human Fcs; bottom panel, human Nb-human Fcs.

FIGS. 43A-43B illustrates an exemplary construction of a Singularity Sapiens-L allelic series designed to include human VL segments. FIG. 43A shows RAG1/RAG2-mediated recombination signal sequences for 12RSS (12 nt spacer) and 23RSS (23 nt spacer) associated with the variable segments at the human loci for IGH, IGK (kappa), and IGL (lambda). (FIG. 43B) A Singularity Sapiens DJ dock allele containing all human D_{H} and J_{H} segments is used as a platform to integrate via sequential RCME a series of BACs containing human variable light chain segments from the human IG Lambda locus of chromosome 22.

The resulting Singularity Sapiens VL-containing allele can produce antibodies that contain a human variable light chain segment contiguous with a human D_{H} segment and a human J_{H} segment followed by a mouse constant region (e.g., a mouse IgG1ΔCH1 region).

FIGS. 44A-44B illustrate an exemplary construction of two distinct sets of Singularity Sapiens-K allelic series expressing human VK segments. FIG. 44A is a schematic showing that a Singularity Hyperdock allele can be used as a platform to integrate via sequential RCME a series of hIGKVJ-BACs containing human VK and JK segments from the human IG Kappa locus on chromosome 2. The resulting Singularity Sapiens VK-JK containing allele can produce antibodies that contain a human variable Kappa segment contiguous with a human Kappa J segment followed by a mouse constant region (e.g., a mouse IgG1ΔCH1 region). FIG. 44B is a schematic showing that a Singularity Sapiens allele containing all human J_{H} segments can be used as a platform to integrate via sequential RCME a series of engineered hIGKV-BACs containing human VK segments from the human IG Kappa locus of chromosome 2. The resulting Singularity Sapiens VK-J_{H}-containing allele can produce antibodies that contain a human variable Kappa segment contiguous with a human J_{H} segment followed by a mouse constant region (e.g., a mouse IgG1ΔCH1 region).

FIGS. 45A-45C illustrate an exemplary engineering design of a Singularity Longhorn. FIG. 45A is a schematic of a genetic construct (Longhorn VDJ) that contains bovine DNA sequences (Bos Taurus) assembled synthetically to include a promoter, a 5′UTR segment, an L1 exon, an intron, an L2 exon of IGHV1-7, the coding segment of IGHD8-2, the coding sequence of IGHJ2-4, and the IGH2-4 splice donor. FIG. 45B is a schematic showing this synthetic construct flanked by disparate loxP elements and a hygromycin selection marker. The construct was integrated into an Igh locus of a Singularity HyperDock allele via RCME to create a Singularity Longhorn allele. FIG. 45C shows a PCR confirmation of mice harboring the Singularity Longhorn allele.

FIG. 46 illustrates an exemplary engineering design of a Singularity Minotaur. Shown is the schematic of a genetic construct (Minotaur DH array) that contains DNA sequences assembled synthetically to include bovine DH segments (e.g., 8 of the longest cow IGVDs, shown inside boxes). To ensure that VDJ recombination occurs, sequences upstream and downstream of the original human IGVD (each labeled below the corresponding bovine IGVD) that contain 12RSS signals were included. This synthetic construct (Minotaur DH array) can be integrated into the Igh locus of a Singularity Sapiens allele (e.g. SSV5) containing any appropriate number (or all) of the human VHs, all human DHs, and all human JHs by, e.g., CRISPR/Cas9 targeting, to replace the human IGVD locus with the synthetic Minotaur DH array.

FIGS. 47A-47B illustrate an exemplary engineering design of a Singularity Sapacos.

FIG. 47A is a schematic of a genetic construct (Sapacos VHH array) containing 5 V_{H}Hs of the alpaca (Vicugna pacos) designed to use human VH elements as genetic scaffolds. Individual VHH elements was grafted onto a framework of a selective human VH that includes an upstream promoter (e.g., a 250 bp upstream promoter) containing regulatory elements (e.g., a TATA box, an octamer, and a heptamer), a leader exon 1, an intron, a leader exon 2, and recombination signal sequences (e.g., 23RSS). FIG. 47B is a schematic showing that this synthetic construct (Sapacos VHH array) containing flanking disparate lox elements and a selection marker can be integrated into the Igh locus of a Singularity Sapiens allele containing all human VD and VJ elements via RMCE.

FIGS. 48A-48B illustrate an exemplary engineering design of a Singularity Savnars. FIG. 48A is a schematic of a genetic construct (Savnars VNAR array) containing two germline VNARs from the nurse shark designed to use human VH elements as genetic scaffolds. Individual VNAR elements was grafted onto a framework of a selective human VH that includes an upstream promotor (e.g., a 250 bp upstream promoter) containing regulatory elements (e.g., a TATA box, an octamer, and a heptamer), a leader exon 1, an intron, a leader exon 2, and recombination signal sequences (e.g., 23RSS). FIG. 48B is a schematic showing that this synthetic construct (Savnars VNAR array) containing flanking disparate lox elements and a selection marker can be integrated into the Igh locus of the Singularity Sapiens allele containing all human VD and VJ elements via RMCE.

### DETAILED DESCRIPTION

This document relates to genetically modified or engineered non-human animals (e.g., genetically modified or engineered mice) that produce heavy chain antibodies (e.g., mouse heavy chain antibodies, humanized heavy chain antibodies, or chimeric heavy chain antibodies) and methods of making the same. For example, this document provides genetically engineered non-human animals of a particular species (e.g., mouse species) that produce heavy chain antibodies of that same species (e.g., mouse heavy chain antibodies). In another example, this document provides genetically engineered non-human animals (e.g., genetically engineered mice) that produce chimeric heavy chain antibodies (e.g., human-mouse chimeric heavy chain antibodies, bovine-human-mouse chimeric heavy chain antibodies, alpaca-human-mouse chimeric heavy chain antibodies, or shark-human-mouse chimeric heavy chain antibodies).

In some cases, heavy chain antibodies obtained or identified from a genetically engineered non-human animal (e.g., a genetically engineered mouse) provided herein can be used to generate single domain antibodies such as mouse single domain antibodies, non-mouse single domain antibodies, humanized single domain antibodies, human single domain antibodies, or chimeric single domain antibodies (e.g., bovine-human chimeric single domain antibodies, alpaca-human chimeric single domain antibodies, or shark-human chimeric single domain antibodies).

This document also relates generally to nanobody compositions and other sources of nanobody compositions from these genetically modified mice. The compositions described herein can be used to treat or prevent a disease or disorder.

As described herein, this document provides methods for producing mammalian single domain antibodies (also known as nanobodies) in vivo. For example, a modified mouse endogenous IgH allele can be constructed such that the constant region C_{H} contains only a CH1-truncated IgG1 gene (IgG1ΔCH1), with all of the other Ig classes or subtypes removed, resulting in the production of only heavy chain IgG1 antibodies. With this modification, the IgG1-ΔCH1 gene is repositioned immediately downstream of the Eμ enhancer, Iμ promoter, Iμ exon, and Sp switch repeat region, with other regulatory elements including the γ1E, 5′hsR1, 3′RR and 3′CBE enhancers kept intact at the endogenous IgH allele. As a result, constitutive high-level expression of IgG1-based heavy chain antibodies (IgG1 HCAb) can be achieved instead of inducible expression from the native regulatory elements for each Ig subtypes, and the entire VH repertoire becomes accessible to produce IgG1 HCAb irrespective of the type of antigens. The engineered non-human (e.g., mouse) endogenous IgH allele described herein can be termed Singularity and can be further modified by removing all non-human (e.g., mouse) endogenous variable exons and introducing a docking site to allow for the replacement of variable exons from human or other mammalian species (or combinations thereof) to produce chimeric antibodies based on the IgG1 HCAb platform, which can be used to derive species-specific single domain antibodies. A highly efficient method is provided herein to introduce long genomic DNA fragments sequentially onto the docking site. As demonstrated herein, these methods can result in the successful generation of a Singularity Sapiens allele that contains 91 human VH exons, to maximize possible antibody diversity. In some cases, variable exons from the IgK and IgL (VK, VL) alleles can be used instead of (or in addition to) VH exons to generate light-chain based single domain antibodies. In a similar fashion, genetic elements corresponding to V_{H} segments, diversity D_{H}, and/or junction JH (or combinations thereof) from other species can be designed and synthesized and placed into the Singularity allele to produce heavy chain antibodies that can be used to make single domain antibodies having unique properties.

The engineered non-human animals (e.g., mice) described herein can exhibit normal B cell development and mount strong humoral immune response upon antigen challenge. A high-throughput sequence-driven approach described herein can be used to produce Ig (e.g., IgG1) HCAb that exhibit high affinities to immunizing antigens. Upon completion of antigen immunization, the entire Ig repertoire can be amplified from lymphoid organs (e.g., a spleen) and subjected to next generation sequencing (NGS) to obtain clonotypes for phylogenetic analyses. Candidate clonotypes can be codon-optimized, synthesized, cloned into expression vectors, and expressed as nanobody-Fc fusions and/or nanobodies in 96-well format. Supematants can be used for ELISA screening to identify antigen specific heavy chain antibodies and/or nanobodies for large scale production, purification, and/or characterization. The purified nanobodies, nanobody-Fc fusions, and/or heavy chain antibodies can exhibit high levels of thermostability, antigen affinity, cell binding, and blocking activities.

As described herein, non-human animals (e.g., mice) can be designed to produce heavy chain only antibodies (HCAbs). In some cases, gene editing (e.g., CRISPR/Cas9) can be used to edit the endogenous IgH allele to generate a Singularity allele (e.g., a Singularity Musculus allele) that contains only an IgG gene (e.g., an Ighg1 gene) in the constant region that encodes a CH1-truncated IgG (e.g., an IgG1-ΔCH1). In some cases, all endogenous genes encoding other antibody isotypes (IgM, IgD, IgE, and IgA) and IgG subtypes (IgG2b, IgG2c, and IgG3) can be eliminated. In some cases, one or more endogenous regulatory elements can be maintained to allow for efficient and faithful transcription of the mutant Ighg1 gene from the endogenous IgH allele. Class switch recombination can thus be abolished in these Singularity non-human animals (e.g., mice) to avoid any potential mechanisms that may compromise the expression of the IgG1-ΔCH1, and to facilitate antibody discovery and purification. The resulting Singularity non-human animals (e.g., Singularity Musculus mice) can be viable and fertile without obvious abnormalities, and they can mount robust humoral immune responses upon antigen challenge and produce IgG1-ΔCH1 heavy chain antibodies with high affinities. Since knowledge of heavy chain-light chain pairing is not required, the much faster and cost-effective NGS-driven antibody discovery pipeline from bulk RNA-sequencing (RNA-seq) analysis of splenocytes described herein can be used to identify antigen-specific monoclonal heavy chain antibodies, where an entire process (e.g., antigen immunization, B cell isolation, bulk sequencing of antibody repertoires, antibody sequence clonotyping, high throughput cloning, expression, and antigen binding assays) can be accomplished within 3 months.

As also described herein, engineered non-human animals (e.g., mice) can be designed to produce human and/or chimeric heavy chain antibodies that can be used to identify therapeutic nanobodies. For example, a Singularity allele (e.g., a Singularity Musculus allele) can be further edited to generate a Singularity HyperDock allele that lacks all mouse VDJ(H) genes and harbors docking sites for sequential introduction of DNA fragments using recombinase-mediated cassette exchange (RMCE). In some cases, clones (e.g., BAC clones) containing human VDJ (H) fragments can be engineered by bacterial homologous recombination (recombineering) to incorporate alternating selection cassettes and heterospecific lox sites with overlapping genomic fragments trimmed off. In some cases, engineered BACs can be used for sequential RMCE to assemble the human VDJ genes upstream of the mouse IgH Ep enhancer in a stepwise fashion. This can result in the generation of a series of Singularity Sapiens alleles (e.g., SSV1-SSV5) with increasing VH diversity until a complete reconstruction of the entire human VDJ genomic region is obtained.

In a similar fashion, a series of Singularity non-human animals (e.g., Singularity mice) can be generated to enable the production of species-specific nanobodies of unique properties for various diagnostic and therapeutic applications.

### Definitions

As used herein, the term “antibody” refers to a molecule that specifically binds to, or is immunologically reactive with, a particular antigen and includes at least the variable domain of a heavy chain and/or light chain, and in some cases can include at least the variable domains of a heavy chain and of a light chain of an immunoglobulin. Antibodies and antigen-binding fragments, variants, or derivatives thereof include, without limitation, polyclonal, monoclonal, multispecific, human, humanized, primatized, or chimeric antibodies, heteroconjugate antibodies (e.g., bi- tri- and quad-specific antibodies, diabodies, triabodies, and tetrabodies), single-domain antibodies (sdAb), epitope-binding fragments, e.g., Fab, Fab′ and F(ab′)₂, Fd, Fvs, single-chain Fvs (scFv), recombinant IgG (rlgG), single-chain antibodies (e.g., heavy chain antibodies or light chain antibodies), disulfide-linked Fvs (sdFv), fragments including either a VL or VH domain, fragments produced by an Fab expression library, and anti-idiotypic (anti-Id) antibodies. Antibody molecules described herein can be of any type (e.g., IgG, IgE, IgM, IgD, IgA, and IgY), class (e.g., IgG1, IgG2, IgG3, IgG4, IgA1 and IgA2), or subclass of immunoglobulin molecule. Moreover, unless otherwise indicated, the term “monoclonal antibody” (mAb) is meant to include both intact molecules as well as antibody fragments (such as, for example, Fab and F(ab′)2 fragments) that are capable of specifically binding to a target protein. Fab and F(ab′)2 fragments lack the Fc fragment of an intact antibody. The term “inhibitory antibody” refers to antibodies that are capable of binding to a target antigen and inhibiting or reducing its function and/or attenuating one or more signal transduction pathways mediated by the antigen. For example, inhibitory antibodies may bind to and block a ligand-binding domain of a receptor, or to extracellular regions of a transmembrane protein. Inhibitory antibody molecules that enter a cell may block the function of an enzyme antigen or signaling molecule antigen. Inhibitory antibodies inhibit or reduce antigen function and/or attenuate one or more antigen-mediated signal transduction pathway by at least 10% (e.g., 10%, 20%, 30%, 40%, 50%, 60%, 70%, 80%, 90%, 95%, 98% or more). The term “agonist antibody” refers to antibodies that are capable of binding to a target antigen and increasing its activity or function, e.g., increasing or activating one or more signal transduction pathways mediated by the antigen. For example, an agonist antibody may bind to and agonize an extracellular region of a transmembrane protein. Agonist antibody molecules that enter a cell may increase the function of an enzyme antigen or signaling molecule antigen. Agonist antibodies activate or increase antigen function and/or one or more antigen-mediated signal transduction pathway by at least 10% (e.g., 10%, 20%, 30%, 40%, 50%, 60%, 70%, 80%, 90%, 95%, 98% or more).

As used herein, the term “antigen” refers to a molecule capable of being bound by an antibody or a T cell receptor (TCR) if presented by MHC molecules. The term “antigen,” as used herein, also encompasses T-cell epitopes. A T-cell epitope is recognized by a T-cell receptor in the context of a MHC class I, present on all cells of the body except erythrocytes, or class II, present on immune cells and in particular antigen presenting cells. This recognition event leads to activation of T-cells and subsequent effector mechanisms such as proliferation of the T-cells, cytokine secretion, perform secretion etc. An antigen is additionally capable of being recognized by the immune system and/or being capable of inducing a humoral immune response and/or cellular immune response leading to the activation of B- and/or T-lymphocytes. This may, however, require that, at least in certain cases, the antigen contains or is linked to a TH cell epitope and is given in adjuvant. An antigen can have one or more epitopes (B- and T-epitopes). The specific reaction referred to above is meant to indicate that the antigen will preferably react, typically in a highly selective manner, with its corresponding antibody or TCR and not with the multitude of other antibodies or TCRs which may be evoked by other antigens. Antigens as used herein may also be mixtures of several individual antigens. Antigens, as used herein, include but are not limited to allergens, self-antigens, haptens, cancer antigens (i.e., tumor antigens) and infectious disease antigens as well as small organic molecules such as drugs of abuse (like nicotine) and fragments and derivatives thereof. Furthermore, antigens used for the present disclosure can be peptides, proteins, domains, carbohydrates, alkaloids, lipids or small molecules such as, for example, steroid hormones and fragments and derivatives thereof, autoantibody and cytokine itself.

“Antigen” also refers to a molecule (e.g., a peptide, protein, or non-peptide) containing one or more epitopes (either linear, conformational, or both) that will stimulate a host's immune system to make a humoral and/or cellular antigen-specific response. The term is used interchangeably with the term “immunogen.” Normally, a B-cell epitope will include at least about 5 amino acids but can be as small as 3-4 amino acids. A T-cell epitope, such as a CTL epitope, will include at least about 7-9 amino acids, and a helper T-cell epitope at least about 12-20 amino acids. Normally, an epitope will include between about 7 and 15 amino acids, such as, 9, 10, 12, or 15 amino acids. The term includes polypeptides which include modifications, such as deletions, additions, and substitutions (generally conservative in nature) as compared to a native sequence, so long as the protein maintains the ability to elicit an immunological response, as defined herein. These modifications may be deliberate, as through site-directed mutagenesis, or may be accidental, such as through mutations of hosts which produce the antigens.

The term “antigen-binding fragment,” as used herein, refers to one or more fragments of an immunoglobulin that retain the ability to specifically bind to a target antigen. The antigen-binding function of an immunoglobulin can be performed by fragments of a full-length antibody. The antibody fragments can be a Fab, F(ab′)₂, scFv, SMIP, diabody, a triabody, an affibody, a nanobody, an aptamer, or a domain antibody. Examples of binding fragments encompassed by the term “antigen-binding fragment” of an antibody include, but are not limited to: (i) a Fab fragment, a monovalent fragment consisting of the VL, VH, CL, and CH1 domains; (ii) a F(ab′)₂ fragment, a bivalent fragment including two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb (Ward et al., Nature, 341:544-546 (1989)) including VH and VL domains; (vi) a dAb fragment that consists of a VH domain; (vii) a dAb that consists of a VH or a VL domain; (viii) an isolated complementarity determining region (CDR); and (ix) a combination of two or more isolated CDRs which may optionally be joined by a synthetic linker. Furthermore, although the two domains of the Fv fragment, VL and VH, are coded for by separate genes, they can be joined, using recombinant methods, by a linker that enables them to be made as a single protein chain in which the VL and VH regions pair to form monovalent molecules (known as single chain Fv (scFv)). These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in certain cases, by chemical peptide synthesis procedures known in the art.

As use herein, the term “antigenic formulation” or “antigenic composition” refers to a preparation which, when administered to a subject, e.g. a mammal, will induce an immune response.

As used herein, the term “biological sample” refers to a specimen (e.g., blood, blood component (e.g., serum or plasma), urine, saliva, amniotic fluid, cerebrospinal fluid, tissue (e.g., a biopsy), pancreatic fluid, chorionic villus sample, and cells) isolated from a subject.

As used herein, a “combination therapy” or “administered in combination” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In other embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be affected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination may be administered by intravenous injection while a second therapeutic agent of the combination may be administered orally.

As used herein, the terms “effective amount,” “therapeutically effective amount,” and a “sufficient amount” of a composition described herein refer to a quantity sufficient to, when administered to a subject, including a mammal (e.g., a human), effect beneficial or desired results, including effects at the cellular level, tissue level, or clinical results, and, as such, an “effective amount” or synonym thereto depends upon the context in which it is being applied.

For example, in the context of treating cancer it is an amount of the composition sufficient to achieve a treatment response as compared to the response obtained without administration of the composition, antibody, vector construct, viral vector, or cell. The amount of a given composition described herein that will correspond to such an amount will vary depending upon various factors, such as the given agent, the pharmaceutical formulation, the route of administration, the type of disease or disorder, the identity of the subject (e.g., age, sex, weight) or host being treated, and the like, but can nevertheless be routinely determined by one skilled in the art. Also, as used herein, a “therapeutically effective amount” of a composition described herein is an amount that results in a beneficial or desired result in a subject as compared to a control. As defined herein, a therapeutically effective amount of a composition described herein may be readily determined by one of ordinary skill by routine methods known in the art. Dosage regimen may be adjusted to provide the optimum therapeutic response.

As used herein, the terms “heavy chain antibody,” “heavy-chain antibody,” “heavy chain-only antibody,” and “HCAb” can be used interchangeably and refer to an antibody that lacks the light chains typically found in conventional antibodies. A heavy chain antibody can be any antibody derived from the immunoglobulin heavy chain (IgH) locus, such as antibodies comprising one or more heavy chain constant domains. For example, a heavy chain antibody can be an antibody comprising one light chain variable domain VL and one or more heavy chain constant domains.

A used herein, the term “hybrid” or “chimeric” refers to a molecule (e.g., protein or VLP) that contains portions thereof, from at least two different proteins. For example, a hybrid influenza HA protein refers to a protein comprising at least a portion of an influenza HA protein (for example a portion containing one or more antigenic determinants) and portions of a heterologous protein (e.g., the cytoplasmic and/or transmembrane domain of a different influenza protein or a different viral protein, for example an RSV or VSV protein). It will be apparent that a hybrid molecule as described herein can include full-length proteins fused to additional heterologous polypeptides (full length or portions thereof) as well as portions of proteins fused to additional heterologous polypeptides (full length or portions thereof). It will also be apparent that the hybrids can include wild-type sequences or mutant sequences in any one, some or all of the heterologous domains.

As used herein, the terms “increasing” and “decreasing” refer to modulating that results in, respectively, greater or lesser amounts, of function, expression, or activity of a metric relative to a reference. For example, subsequent to administration of an antibody described herein, the amount of a marker of a metric (e.g., cancer cell death or DNA methylation of a target site) as described herein may be increased or decreased in a subject by at least 5%, 10%, 15%, 20%, 25%, 30%, 35%, 40%, 45%, 50%, 55%, 60%, 65%, 70%, 75%, 80%, 85%, 90%, 95% or 98% or more relative to the amount of the marker prior to administration. Generally, the metric is measured subsequent to administration at a time that the administration has had the recited effect, e.g., at least one week, one month, 3 months, or 6 months, after a treatment regimen has begun.

An “immunological response” to an antigen or composition is the development in a subject of a humoral and/or a cellular immune response to an antigen present in the composition of interest. For purposes of this document, a “humoral immune response” refers to an immune response mediated by antibody molecules, while a “cellular immune response” is one mediated by T-lymphocytes and/or other white blood cells. One important aspect of cellular immunity involves an antigen-specific response by cytolytic T-cells (CTLs). CTLs have specificity for peptide antigens that are presented in association with proteins encoded by the major histocompatibility complex (MHC) and expressed on the surfaces of cells.

CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+ T-cells.

Hence, an immunological response may include one or more of the following effects: the production of antibodies by B-cells; and/or the activation of suppressor T-cells and/or γδ T-cells directed specifically to an antigen or antigens present in the composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art.

An “immunogenic composition” is a composition that comprises an antigenic molecule where administration of the composition to a subject results in the development in the subject of a humoral and/or a cellular immune response to the antigenic molecule of interest.

As used herein, the term “multivalent” refers to a compound which has multiple antigenic proteins against multiple types or strains of infectious agents, such as antigen, antibody, or virus-like particle (VLP).

As used herein, a “particle-forming polypeptide” can be derived from a particular viral protein, including a full-length or near full-length viral protein, as well as a fragment thereof, or a viral protein with internal deletions, which has the ability to form VLPs under conditions that favor VLP formation. Accordingly, the polypeptide may comprise the full-length sequence, fragments, truncated and partial sequences, as well as analogs and precursor forms of the reference molecule. The term therefore includes deletions, additions and substitutions to the sequence, so long as the polypeptide retains the ability to form a VLP. Thus, the term includes natural variations of the specified polypeptide since variations in coat proteins often occur between viral isolates. The term also includes deletions, additions and substitutions that do not naturally occur in the reference protein, so long as the protein retains the ability to form a VLP. Preferred substitutions are those which are conservative in nature, i.e., those substitutions that take place within a family of amino acids that are related in their side chains. Specifically, amino acids are generally divided into four families: (1) acidic-aspartate and glutamate; (2) basic-lysine, arginine, histidine; (3) non-polar-alanine, valine, leucine, isoleucine, proline, phenylalanine, methionine, tryptophan; and (4) uncharged polar-glycine, asparagine, glutamine, cysteine, serine threonine, tyrosine. Phenylalanine, tryptophan, and tyrosine are sometimes classified as aromatic amino acids.

As used herein, the terms “light chain variable region” and “heavy chain variable region” refer to the variable binding region from an antibody light and heavy chain, respectively. The variable binding regions are made up of discrete, well-defined sub-regions known as “complementarity determining regions” (CDRs) and “framework regions” (FRs), generally comprising in order FR1-CDR1-FR2-CDR2-FR3-CDR3-FR4 from amino-terminus to carboxyl-terminus. In one embodiment, the FRs are humanized. The term “CL” refers to an “immunoglobulin light chain constant region” or a “light chain constant region,” i.e., a constant region from an antibody light chain. The term “CH” refers to an “immunoglobulin heavy chain constant region” or a “heavy chain constant region,” which is further divisible, depending on the antibody isotype into CH1, hinge, CH2, and CH3 (for IgA, IgD, and IgG), or CH1, CH2, CH3, and CH4 domains (for IgE and IgM).

As used herein, a “pharmaceutical composition” or “pharmaceutical preparation” is a composition or preparation having pharmacological activity or other direct effect in the mitigation, treatment, or prevention of disease, and/or a finished dosage form or formulation thereof and which is indicated for human use.

As used herein, the term “reference” refers to a level, expression level, copy number, sample, or standard that is used for comparison purposes. For example, a reference sample can be obtained from a healthy individual (e.g., an individual who does not have cancer). A reference level can be the level of expression of one or more reference samples. For example, an average expression (e.g., a mean expression or median expression) among a plurality of individuals (e.g., healthy individuals, or individuals who do not have cancer) can be a reference level. In other instances, a reference level can be a predetermined threshold level, e.g., based on functional expression as otherwise determined, e.g., by empirical assays.

As used herein, the terms “subject” and “patient” refer to an animal (e.g., a mammal, such as a human). A subject to be treated according to the methods described herein may be one who has been diagnosed with a particular condition, or one at risk of developing such conditions. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

“Treatment” and “treating,” as used herein, refer to the medical management of a subject with the intent to improve, ameliorate, stabilize (i.e., not worsen), prevent or cure a disease, pathological condition, or disorder. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to minimizing or partially or completely inhibiting the development of the associated disease, pathological condition, or disorder), and supportive treatment (treatment employed to supplement another therapy). Treatment also includes diminishment of the extent of the disease or condition; preventing spread of the disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

It should be understood that for all numerical bounds describing some parameter in this document, such as “about,” “at least,” “less than,” and “more than,” the description also necessarily encompasses any range bounded by the recited values. Accordingly, for example, the description “at least 1, 2, 3, 4, or 5” also describes, inter alia, the ranges 1-2, 1-3, 1-4, 1-5, 2-3, 2-4, 2-5, 3-4, 3-5, and 4-5, et cetera.

It should also be understood that the terms “a” and “an” as used herein refer to “one or more” of the enumerated components unless otherwise indicated. The use of the alternative (e.g., “or”) should be understood to mean either one, both, or any combination thereof of the alternatives.

### Singularity Non-Human Animals (e.g., Singularity Mice)

This document provides genetically engineered non-human animals (e.g., non-human mammals such as mice) for producing antibodies (e.g., heavy chain antibodies such as mouse heavy chain antibodies or chimeric heavy chain antibodies). For example, genetically engineered non-human animals for producing heavy chain antibodies can be non-human animals having (e.g., engineered to have) humanized IgG heavy chains. In some cases, non-human animals for producing heavy chain antibodies (e.g., heavy chain antibodies such as mouse heavy chain antibodies or chimeric heavy chain antibodies) can be non-human animals whose genomes have (e.g., are genetically engineered to have) one or more disruptions in an endogenous nucleic acid sequence encoding an CH1 domain of an IgG1 C-region gene (e.g., C_{γ1}). In some cases, a non-human animal (e.g., a mouse) can be designed to produce heavy chain antibodies (e.g., heavy chain antibodies such as mouse heavy chain antibodies or chimeric heavy chain antibodies) that lack CH1 domains and that lack light chains. Also provided herein are methods and materials for making and using non-human animals described herein.

In some cases, one or more genetic germline modifications can be carried out to create a non-human animal described herein. The genetic modification(s) can cause the non-human animal (e.g., mouse) to express IgG heavy chain antibodies and secrete the IgG heavy chain antibodies into its serum. In some cases, the IgG heavy chain antibodies can be humanized. For example, the variable region of an IgG heavy chain antibody can be a human variable region and the constant region of the IgG heavy chain antibody can be a mouse constant region. In some cases, a non-human animal (e.g., mouse) described herein can be designed to produce IgG1 heavy chain antibodies, IgG2 heavy chain antibodies, IgG3 heavy chain antibodies, or IgG4 heavy chain antibodies. In some cases, a non-human animal (e.g., mouse) described herein can be designed to produce any combination of two or more of (a) IgG1 heavy chain antibodies, (b) IgG2 heavy chain antibodies, (c) IgG3 heavy chain antibodies, and (d) IgG4 heavy chain antibodies.

In some cases, a non-human animal provided herein can be designed to have a deletion of nucleic acid encoding a CH1 domain of an IgG C-region (e.g., a CH1 domain of an IgG1 C-region, a CH1 domain of an IgG2a C-region, a CH1 domain of an IgG2b C-region, and/or a CH1 domain of an IgG3 C-region). The CH1 domain can contain multiple exons. In some cases, exon 1 of a CH1 domain of an IgG C-region can be deleted such that the engineered non-human animal (e.g., mouse) produces IgGΔCH1 heavy antibodies.

When making one or more genetic modifications to delete all or part of the nucleic acid encoding a CH1 domain (e.g., a CH1 domain of an IgG1 C-region, a CH1 domain of an IgG2a C-region, a CH1 domain of an IgG2b C-region, and/or a CH1 domain of an IgG3 C-region) such that the engineered non-human animal produces IgGΔCH1 heavy chain antibodies, the endogenous nucleic acid encoding a hinge domain, a heavy chain CH2 domain, and a heavy chain CH3 domain can remain intact. For example, to make a mouse that produces IgG1ΔCH1 heavy chain antibodies, the genome of that mouse can lack exon 1 (and/or additional portions) of the CH1 domain of IgG1 while retaining the endogenous mouse nucleic acid needed to express the hinge domain, a heavy chain CH2 domain, and a heavy chain CH3 domain of IgG1, thereby resulting in a mouse that is capable of producing IgG1ΔCH1 heavy chain antibodies.

Additional endogenous nucleic acid components that can be deleted from the genome of a non-human animal (e.g. a mouse) to create a non-human animal provided herein include, without limitation, the introns and/or exons of the IgM constant domains (e.g., the μ constant domain locus), the introns and/or exons of the IgD constant domains (e.g., the 6 constant domain locus), the introns and/or exons of the IgE constant domains (e.g., the E constant domain locus), and/or the introns and/or exons of the IgA constant domains (e.g., the a constant domain locus). For example, a non-human animal provided herein can be designed to lack the introns and exons of the IgM constant domains (e.g., the μ constant domain locus), the introns and exons of the IgD constant domains (e.g., the 6 constant domain locus), the introns and exons of the IgE constant domains (e.g., the E constant domain locus), and the introns and exons of the IgA constant domains (e.g., the a constant domain locus).

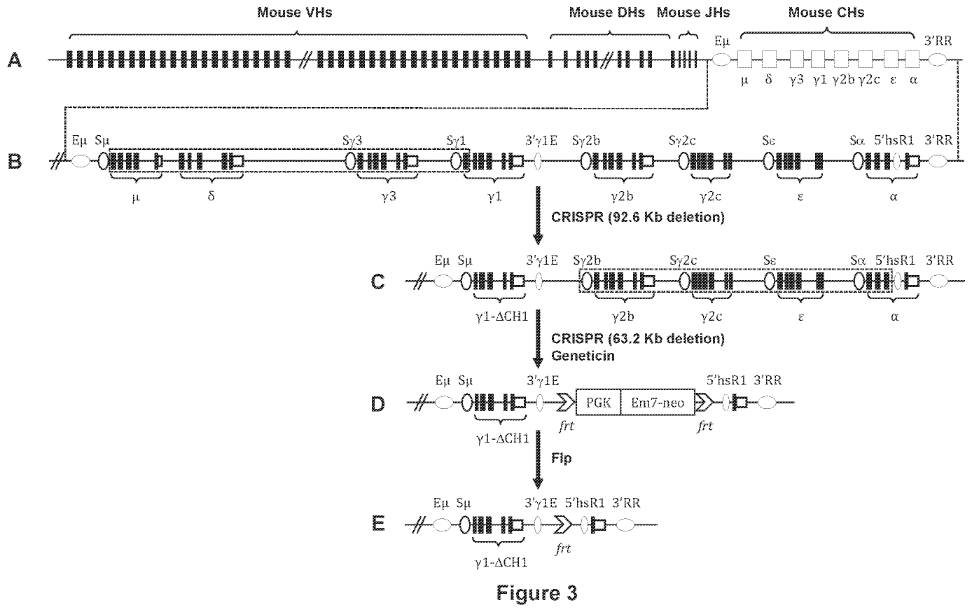

In some cases, when designing a non-human animal (e.g. a mouse) to produce only IgG1ΔCH1 heavy chain antibodies, the genome of that non-human animal can be designed to lack (in addition to lacking endogenous introns and/or exons of the μ constant domain locus, endogenous introns and/or exons of the 6 constant domain locus, endogenous introns and/or exons of the E constant domain locus, and endogenous introns and/or exons the of α constant domain locus) the endogenous (if endogenously present) introns and/or exons of the Igγ3 constant domains (e.g., the γ3 constant domain locus), the endogenous (if endogenously present) introns and/or exons of the Igγ2a constant domains (e.g., the γ2a constant domain locus), the endogenous (if endogenously present) introns and/or exons of the Igγ2b constant domains (e.g., the γ2b constant domain locus), and the endogenous (if endogenously present) introns and/or exons of the Igγ2c constant domains (e.g., the γ2c constant domain locus). An example of a genetic engineering approach to create a mouse that produces only IgG1ΔCH1 heavy chain antibodies is set forth in FIGS. 3A-3E.

In some cases, when designing a non-human animal (e.g. a mouse) to produce only IgG2aΔCH1 heavy chain antibodies, the genome of that non-human animal can be designed to lack (in addition to lacking endogenous introns and/or exons of the μ constant domain locus, endogenous introns and/or exons of the 6 constant domain locus, endogenous introns and/or exons of the E constant domain locus, and endogenous introns and/or exons the of a constant domain locus) the endogenous (if endogenously present) introns and/or exons of the Igγ3 constant domains (e.g., the γ3 constant domain locus), the endogenous (if endogenously present) introns and/or exons of the Igγ1 constant domains (e.g., the γ1 constant domain locus), the endogenous (if endogenously present) introns and/or exons of the Igγ2b constant domains (e.g., the γ2b constant domain locus), and the endogenous (if endogenously present) introns and/or exons of the Igγ2c constant domains (e.g., the γ2c constant domain locus).

In some cases, when designing a non-human animal (e.g. a mouse) to produce only IgG2bACH1 heavy chain antibodies, the genome of that non-human animal can be designed to lack (in addition to lacking endogenous introns and/or exons of the μ constant domain locus, endogenous introns and/or exons of the 6 constant domain locus, endogenous introns and/or exons of the E constant domain locus, and endogenous introns and/or exons the of a constant domain locus) the endogenous (if endogenously present) introns and/or exons of the Igγ3 constant domains (e.g., the γ3 constant domain locus), the endogenous (if endogenously present) introns and/or exons of the Igγ2a constant domains (e.g., the γ2a constant domain locus), the endogenous (if endogenously present) introns and/or exons of the Igγ1 constant domains (e.g., the γ1 constant domain locus), and the endogenous (if endogenously present) introns and/or exons of the Igγ2c constant domains (e.g., the γ2c constant domain locus).

In some cases, when designing a non-human animal (e.g. a mouse) to produce only IgG2ϵACH1 heavy chain antibodies, the genome of that non-human animal can be designed to lack (in addition to lacking endogenous introns and/or exons of the μ constant domain locus, endogenous introns and/or exons of the 6 constant domain locus, endogenous introns and/or exons of the E constant domain locus, and endogenous introns and/or exons the of a constant domain locus) the endogenous (if endogenously present) introns and/or exons of the Igγ3 constant domains (e.g., the γ3 constant domain locus), the endogenous (if endogenously present) introns and/or exons of the Igγ2a constant domains (e.g., the γ2a constant domain locus), the endogenous (if endogenously present) introns and/or exons of the Igγ2b constant domains (e.g., the γ2b constant domain locus), and the endogenous (if endogenously present) introns and/or exons of the Igγ1 constant domains (e.g., the γ1 constant domain locus).

In some cases, when designing a non-human animal (e.g. a mouse) to produce only IgG3ΔCH1 heavy chain antibodies, the genome of that non-human animal can be designed to lack (in addition to lacking endogenous introns and/or exons of the μ constant domain locus, endogenous introns and/or exons of the 6 constant domain locus, endogenous introns and/or exons of the E constant domain locus, and endogenous introns and/or exons the of a constant domain locus) the endogenous (if endogenously present) introns and/or exons of the Igγ1 constant domains (e.g., the γ1 constant domain locus), the endogenous (if endogenously present) introns and/or exons of the Igγ2a constant domains (e.g., the γ2a constant domain locus), the endogenous (if endogenously present) introns and/or exons of the Igγ2b constant domains (e.g., the γ2b constant domain locus), and the endogenous (if endogenously present) introns and/or exons of the Igγ2c constant domains (e.g., the γ2c constant domain locus).

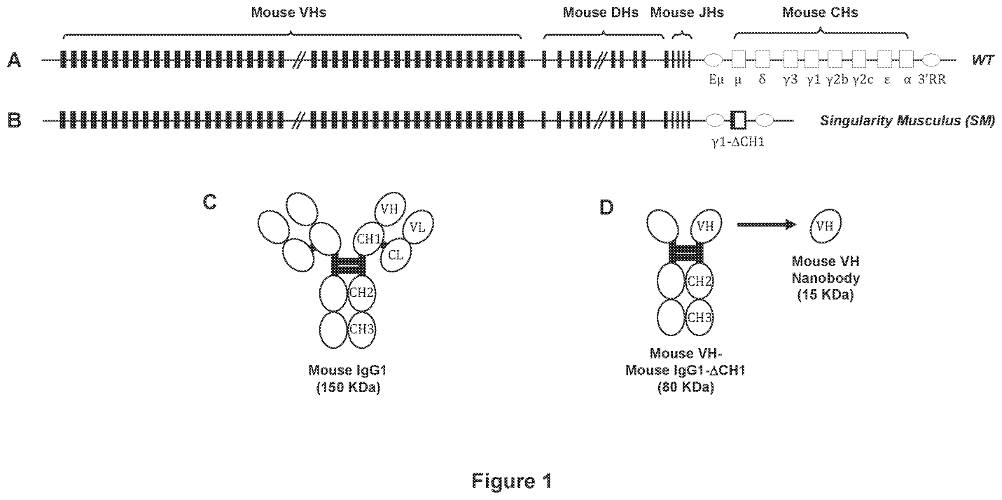

As described herein, retaining and/or creating new positioning for certain endogenous enhancer or regulatory elements of a non-human animal can result in a non-human animal (e.g., mouse) provided herein that produces effectively large collections of and amounts of diverse heavy chain antibodies (e.g., heavy chain antibodies such as mouse heavy chain antibodies or chimeric heavy chain antibodies). For example, non-human animals (e.g., mice) provided herein can be designed to retain the μ enhancer (Eμ), the μ switch region (Sμ), and/or the μ promoter containing I-exon (Iμ) that are endogenously found upstream of the nucleic acid encoding the IgM constant domains. In some cases, non-human animals (e.g., mice) provided herein can be designed such that the retained endogenous Eμ, Sμ, and/or Iμ elements are in a genomic position such that the first nucleic acid sequence downstream of the retained Eμ, Sμ, and/or Iμ elements that encodes a full-length endogenous Ig constant domain is one that encodes a CH2 domain (e.g., nucleic acid that encodes a full length IgG1 CH2 domain, nucleic acid that encodes a full length IgG2a CH2 domain, nucleic acid that encodes a full length IgG2b CH2 domain, nucleic acid that encodes a full length IgG2c CH2 domain, or nucleic acid that encodes a full length IgG3 CH2 domain). An example of this genomic configuration is set forth in FIG. 1B and FIG. 3C where the nucleic acids of the endogenous mouse Eμ, Sμ, and Iμ elements are repositioned to be upstream of the nucleic acid encoding the endogenous IgG1 CH2 domain.

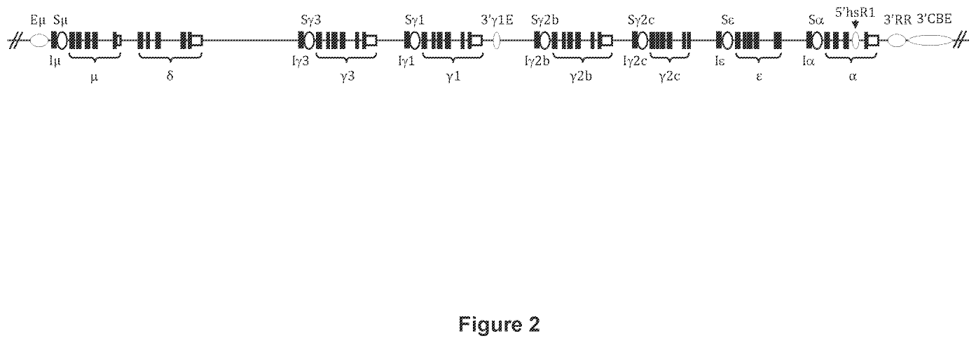

In another example, non-human animals (e.g., mice) provided herein can be designed to retain the 3′RR and/or 3′CBE elements that are endogenously found downstream of the nucleic acid encoding the IgA constant domains. In some cases, non-human animals (e.g., mice) provided herein can be designed such that the retained endogenous 3′RR and/or 3′CBE elements are in a genomic position such that the first nucleic acid sequence upstream of the retained 3′RR and/or 3′CBE elements that encodes a full-length endogenous Ig CH2 constant domain is one that encodes an IgG CH2 domain (e.g., nucleic acid that encodes a full length IgG1 CH2 domain, nucleic acid that encodes a full length IgG2a CH2 domain, nucleic acid that encodes a full length IgG2b CH2 domain, nucleic acid that encodes a full length IgG2c CH2 domain, or nucleic acid that encodes a full length IgG3 CH2 domain). An example of this genomic configuration is set forth in FIG. 2B and FIG. 3E where the nucleic acid of the endogenous mouse 3′RR element is repositioned to be downstream of the nucleic acid encoding the endogenous IgG1 CH2 domain such that no other nucleic acid encoding a full length IgG CH2 domain is located between nucleic acid encoding the endogenous IgG1 CH2 domain and the nucleic acid of the endogenous mouse 3′RR element.

In some cases, non-human animals (e.g., mice) provided herein can be designed to retain the 3′γ1E element that is endogenously found between, for example, the IgG1 and IgG2b loci. In some cases, non-human animals (e.g., mice) provided herein can be designed such that the retained endogenous 3′γ1E element is in a genomic position such that nucleic acid encoding two, one, or no full-length endogenous Ig CH2 domains is located between the retained endogenous 3′γ1E element and a retained endogenous 3′RR element and/or a retained endogenous 3′CBE element. An example of this genomic configuration is set forth in FIG. 3E where the nucleic acid of the endogenous mouse 3′γ1E element is repositioned to be upstream of a retained endogenous 3′RR element such that no other nucleic acid encoding a full length IgG CH2 domain is located between the endogenous mouse 3′γ1E element and the endogenous 3′RR element.

In some cases, non-human animals (e.g., mice) provided herein can be designed to retain the 5′hsR1 element that is endogenously found within the IgA constant domain locus. In some cases, non-human animals (e.g., mice) provided herein can be designed such that the retained endogenous 5′hsR1 element is in a genomic position such that the first nucleic acid sequence upstream of the retained 5′hsR1 element that encodes a full-length endogenous Ig CH2 constant domain is one that encodes an IgG CH2 domain (e.g., nucleic acid that encodes a full length IgG1 CH2 domain, nucleic acid that encodes a full length IgG2a CH2 domain, nucleic acid that encodes a full length IgG2b CH2 domain, nucleic acid that encodes a full length IgG2c CH2 domain, or nucleic acid that encodes a full length IgG3 CH2 domain). An example of this genomic configuration is set forth in FIG. 3E where the nucleic acid of the endogenous mouse 5′hsR1 element is repositioned to be downstream of the nucleic acid encoding the endogenous IgG1 CH2 domain such that no other nucleic acid encoding a full length IgG CH2 domain is located between nucleic acid encoding the endogenous IgG1 CH2 domain and the nucleic acid of the endogenous mouse 5′hsR1 element.

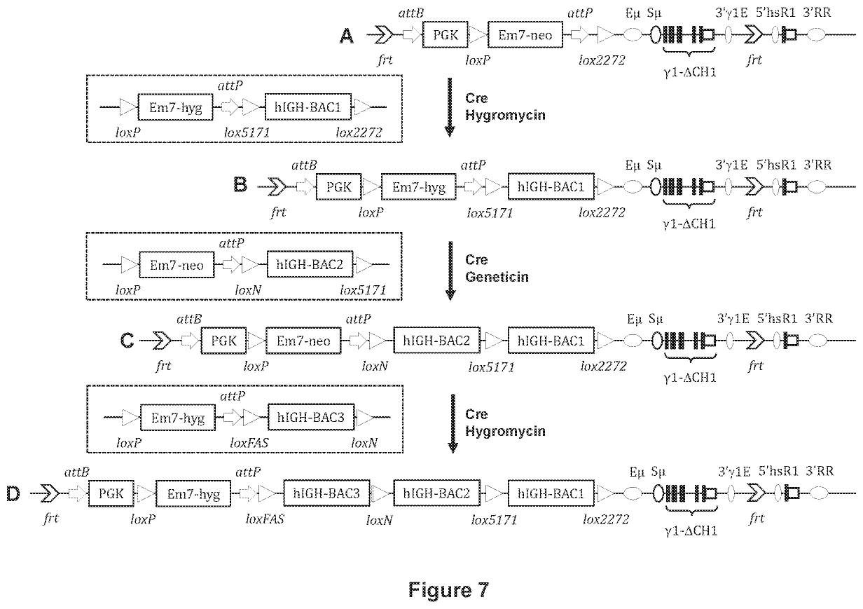

In some cases, non-human animals (e.g., mice) provided herein can be designed to have (a) a variable region locus (e.g., a mouse variable region locus, a non-mouse variable region locus, a human variable region locus, or a chimeric variable region locus such as a bovine-human chimeric variable region locus, an alpaca-human chimeric variable region locus, or a shark-human chimeric variable region locus) followed by (b) an endogenous Eμ element and/or an endogenous element Iμ and/or an endogenous Sp element followed by (c) nucleic acid that encodes endogenous IgG hinge, CH2, and CH3 domains in the absence of the endogenous CH1 domain for that IgG followed by (e) an endogenous 3′γ1E element, an endogenous 3′RR element, and an endogenous 3′CBE element, while lacking the endogenous nucleic acid that encodes at least one full-length CH2 or CH3 domain of each of IgM, IgD, IgE, and IgA. An example of this genomic configuration is set forth in FIG. 3E. See, also, FIGS. 7, 8, 43B, 44, 45B, 47B, and 48B.

In some cases, instead of retaining an endogenous enhancer or regulatory element as described herein, one or more exogenous enhancer or regulatory elements can be engineered into the non-human animal (e.g., mouse). For example, in some cases, a mouse can be designed as described herein where the endogenous mouse Ep element is removed and replaced with a human Ep element.

In some cases, an engineered non-human animal provided herein can be designed to have a variable region locus that is the endogenous variable region locus of that non-human animal. For example, an engineered mouse provided herein can be designed to have the endogenous mouse variable region locus. An example of an IgH locus of such an engineered mouse is set forth in FIG. 1B.

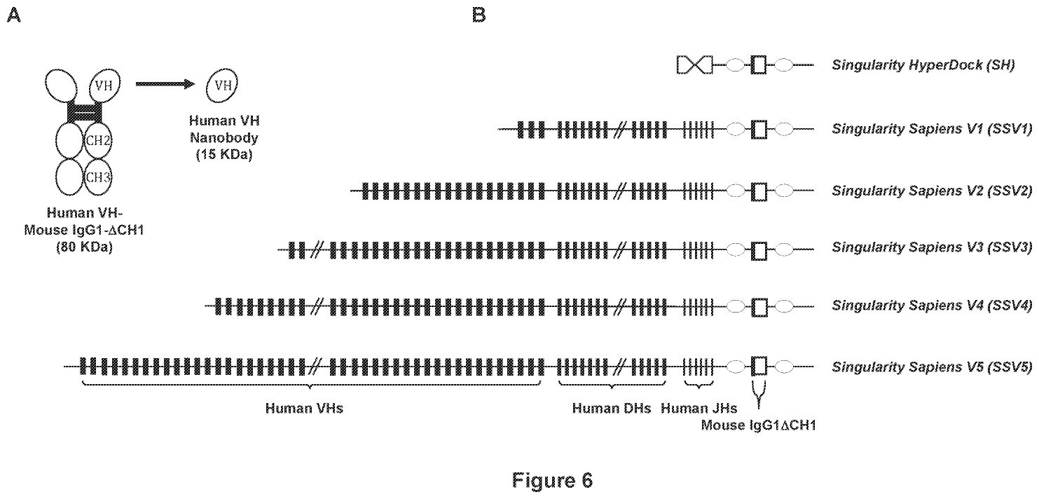

In some cases, an engineered non-human animal provided herein can be designed to have a variable region locus that is not endogenous to that non-human animal. For example, an engineered mouse provided herein can be designed to have a non-mouse variable region locus (e.g., a human variable region locus, an alpaca variable region locus, a shark variable region locus, a bovine variable region locus, a goat variable region locus, a sheep variable region locus, a dog variable region locus, a cat variable region locus, a rat variable region locus, a chicken variable region locus, or a rabbit variable region locus). An example of an IgH locus of such an engineered mouse is set forth in FIG. 6B.

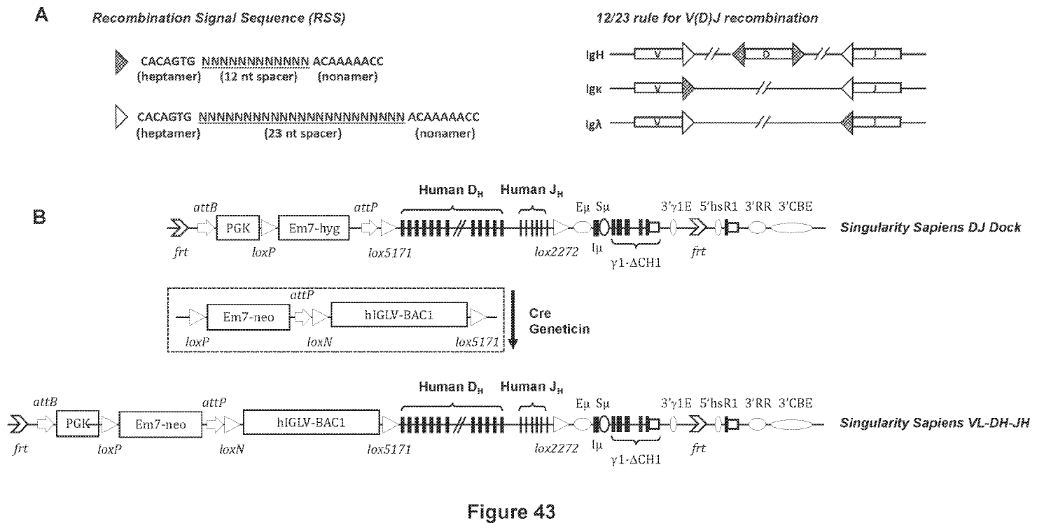

In some cases, an engineered non-human animal provided herein can be designed to have a variable region locus that is not endogenous to that non-human animal such that it includes variable region components from two or more different species that are different from that of the non-human animal. For example, an engineered mouse provided herein can be designed to have a non-mouse variable region locus that includes variable region components of human and alpaca, human and bovine, human and shark, shark and bovine, alpaca and bovine, human and goat, human and sheep, human and dog, human and cat, human and rat, human and chicken, or human and rabbit. Examples of IgH loci of such engineered mice are set forth in FIGS. 43, 44, 47, and 48.

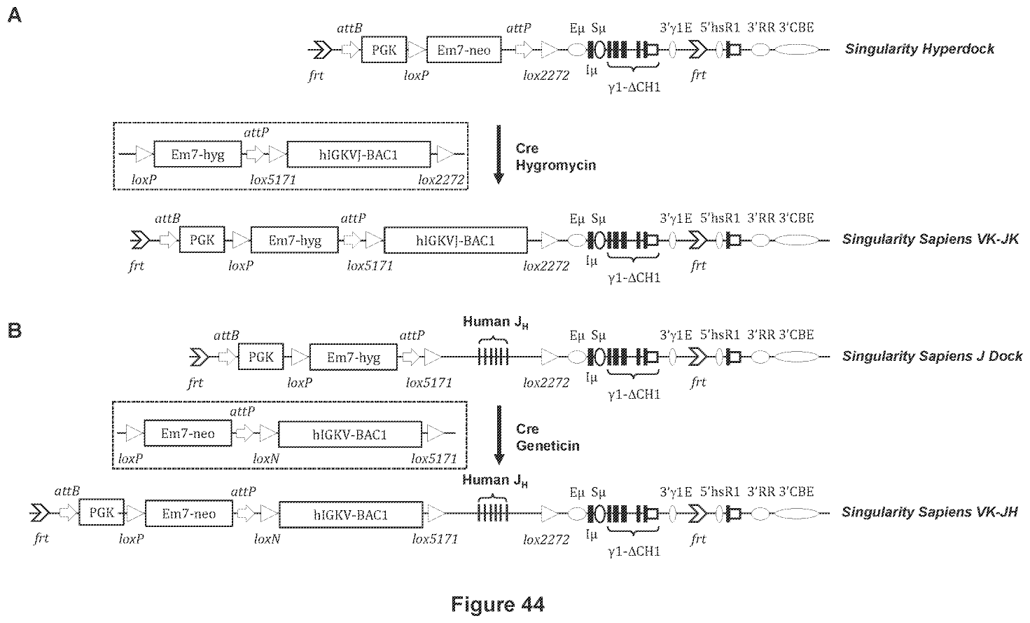

In some cases, an engineered non-human animal provided herein can be designed to have a variable region locus that is a variable region locus of a light chain (e.g., a kappa light chain locus variable region or a lambda light chain locus variable region) as opposed to a variable region locus of a heavy chain. For example, an engineered mouse provided herein can be designed to have a variable region locus of a light chain locus (e.g., a human variable region locus of a kappa or lambda light chain). Examples of IgH loci of such engineered mice are set forth in FIGS. 43B, 44A, and 44B.

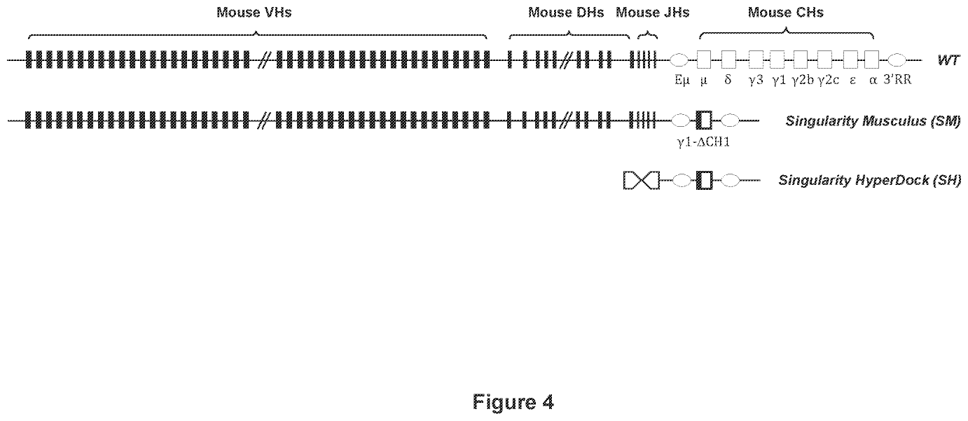

This document also provides engineered non-human animals that can be used to create a non-human animal provided herein that produce antibodies (e.g., heavy chain antibodies lacking CH1 domains). For example, this document provides engineered non-human animals that lack the entire set of exons of the endogenous variable region of the heavy chain locus and contain a cloning nucleic acid segment in its location upstream of the already engineered constant region as described herein. An example of an IgH locus of such an engineered mouse is set forth in FIGS. 4, 5D, and 6B, which can be referred to as a non-human Singularity HyperDock animal or a Singularity HyperDock mouse. Any appropriate cloning nucleic acid segment can be used to make a non-human Singularity HyperDock animal (e.g., a Singularity HyperDock mouse) provided herein. For example, a cloning nucleic acid segment designed to contain one, two, three, four, or more recombinase site recognition sequences (see, e.g., Table 1) can be used to make a non-human Singularity HyperDock animal (e.g., a Singularity HyperDock mouse) provided herein. In some cases, a non-human Singularity HyperDock animal (e.g., a Singularity HyperDock mouse) provided herein lacks the ability to produce any Ig heavy chains.

TABLE 1
Exemplary recombination site
recognition sequences.

| | 13 bp | 8 bp | 13 bp |
|---|---|---|---|
| | Recognition | Spacer | Recognition |
| Lox Site | Region | Region | Region |
| loxP | ATAACTTCGTATA | GCATACAT | TATACGAAGTTAT |
| (Wildtype) | | | |
| lox2272 | ATAACTTCGTATA | GgATACIT | TATACGAAGTTAT |
| lox5171 | ATAACTTCGTATA | GtAcACAT | TATACGAAGTTAT |
| loxN | ATAACTTCGTATA | GtATACcT | TATACGAAGTTAT |
| loxFAS | ATAACTTCGTATA | GaAaggta | TATACGAAGTTAT |
| lox2372 | ATAACTTCGTATA | GgATACcT | TATAGGRAGTTAT |
| M2 | ATAACTTCGTATA | tggTttcT | TATACGAAGTTAT |
| M3 | ATAACTTCGTATA | taATACcg | TATACGAAGTTAT |
| M7 | ATAACTTCGTATA | ttcTAtcT | TATACGAAGTTAT |
| M11 | ATAACtTCGTATA | agATAgaa | TATACGAAGTTAT |
| lox | ATAACTTCGTATA | GCATACAT | TATACGAAcggta |
| lox73 | taccgTTCGTATA | GCATACAT | TATACGAAGTTAT |
| lox72 | taccgTTCGTATA | GCATACAT | TATACGAAcggTa |
| lox511 | ATAACTTCGTATA | GtATACAT | TATACGAAGTTAT |
| loxBri | ATAACTTCGTATA | aacTAtAC | TATACGAAGTTAT |

Any appropriate method can be used to make a non-human animal provided herein (e.g., a non-human animal designed to produce heavy chain antibodies such as IgG1ΔCH1 heavy chain antibodies as described herein and a non-human Singularity HyperDock animal such as a Singularity HyperDock mouse describe herein). For example, gene editing techniques (e.g., CRISPR/Cas gene editing, TALEN gene editing, and/or zinc finger-based gene editing), recombination techniques (e.g., sequential Recombinase Mediated Cassette Exchange (RMCE)), and combinations thereof can be used to make a non-human animal provided herein. In some cases, the genetic engineering techniques described in the Examples can be used to make a non-human animal provided herein.

This document also provides human nanobodies, humanized nanobodies, heavy chain antibodies lacking CH1 domains (e.g., fully mouse heavy chain antibodies lacking CH1 domains), and chimeric heavy chain antibodies (e.g., human-mouse chimeric heavy chain antibodies with or without CH1 domains). For example, this document provides fully human nanobodies produced or derived from a non-human animal described herein. As another example, this document provides fully mouse heavy chain antibodies lacking CH1 domains. As another example, this document provides chimeric heavy chain antibodies. Such chimeric heavy chain antibodies can lack CH1 domains as described herein. In some cases, chimeric heavy chain antibodies provided herein (e.g., IgGΔCH1 heavy chain antibodies) can contain one or more variable region components that are human, alpaca, shark, bovine, goat, sheep, dog, cat, rat, chicken, or rabbit and constant region components of a different species (e.g., a mouse). For example, chimeric heavy chain antibodies provided herein (e.g., IgGΔCH1 heavy chain antibodies) can have a human variable region and a mouse constant region. In some cases, a chimeric heavy chain antibody provided herein (e.g., an IgGΔCH1 heavy chain antibody) can have an alpaca variable region and a mouse constant region. In some cases, a chimeric heavy chain antibody provided herein (e.g., an IgGΔCH1 heavy chain antibody) can have a shark variable region and a mouse constant region. In some cases, a chimeric heavy chain antibody provided herein (e.g., an IgGΔCH1 heavy chain antibody) can have a bovine variable region and a mouse constant region. In some cases, a chimeric heavy chain antibody provided herein (e.g., an IgGΔCH1 heavy chain antibody) can have a variable region at least partially of alpaca and at least partially of human and a mouse constant region. In some cases, a chimeric heavy chain antibody provided herein (e.g., an IgGΔCH1 heavy chain antibody) can have a variable region at least partially of shark and at least partially of human and a mouse constant region. In some cases, a chimeric heavy chain antibody provided herein (e.g., an IgGΔCH1 heavy chain antibody) can have a variable region at least partially of bovine and at least partially of human and a mouse constant region.

Human nanobodies, humanized nanobodies, heavy chain antibodies lacking CH1 domains (e.g., fully mouse heavy chain antibodies lacking CH1 domains), and chimeric heavy chain antibodies (e.g., human-mouse chimeric heavy chain antibodies with or without CH1 domains) provided herein can be obtained using any appropriate method. For example, heavy chain antibodies provided herein can be obtained from the plasma of a non-human animal provided herein. In some cases, human nanobodies, humanized nanobodies, heavy chain antibodies lacking CH1 domains (e.g., fully mouse heavy chain antibodies lacking CH1 domains), and chimeric heavy chain antibodies (e.g., human-mouse chimeric heavy chain antibodies with or without CH1 domains) provided herein can be obtained using a nucleic acid vector designed to express a nanobody or heavy chain antibody based or derived from a heavy chain antibody produced by a non-human animal provided herein. For example, a human-mouse IgGΔCH1 heavy chain antibody produced by a non-human animal provided herein can be identified as having the ability to bind a target antigen of interest (e.g., a SARS-CoV-2 antigen) and can be sequenced. That sequence can be used to design a nucleic acid vector having the ability to express that same human-mouse IgGΔCH1 heavy chain antibody or the human variable region of that same human-mouse IgGΔCH1 heavy chain antibody as a human nanobody. In some cases, that sequence can be used to design a nucleic acid vector that can express a fully human full length heavy chain antibody that can be used by itself or that can be combined with a fully human light chain to create full tetrameric antibodies.

In some cases, a non-human animal provided herein can be immunized with an antigen of interest (e.g., a SARS-CoV-2 antigen) such that the non-human animal produces antibodies against that antigen. Nucleic acid encoding the produced heavy chain antibodies (e.g., a heavy chain antibody lacking a CH1 domain) can be isolated. For example, amplification techniques such as PCR or 5′RACE can be used to obtain large collections of nucleic acid encoding at least part of the variable region (e.g., one or more CDRs, all three CDRs, or the entire variable region) of different heavy chain antibodies that the non-human animal produced. The isolated nucleic acid sequences can be cloned (with or without prior sequencing) into expression vectors to express the obtained nucleic acid sequences in the context of any appropriate type of antibody (e.g., a nanobody, a heavy chain antibody, or a full antibody) that can be assessed for the desired properties (e.g., binding properties, neutralization properties, and/or solubility properties). Those nucleic acid sequences having the ability to encode an antibody having the desired properties can be used to create any type of antibody such as a nanobody.

Plasma comprising nanobodies can be collected from a subject, or from a non-human animal having a humanized immune system which may have been immunized with an antigen described herein. The nanobodies from a non-human animal having a humanized immune system can be used in treatment of a human subject in need thereof.

In some cases, plasma comprising chimeric heavy chain antibodies that can be used to generate nanobodies as described herein can be collected from a subject, or from a non-human animal provided herein (e.g., a non-human animal having a humanized immune system) which may have been immunized with an antigen as described herein.

Plasma comprising nanobodies can be collected, e.g., via plasmapheresis. In some cases, plasma comprising chimeric heavy chain antibodies described herein can be collected, e.g., via plasmapheresis. Plasma can be collected from the same subject multiple times, for example, multiple times each a given period of time after an immunization, multiple times after an immunization, multiple times in between immunizations, or any combination thereof.

Plasma can be collected from the non-human animal or from a human subject described herein any suitable amount of time following an immunization, for example the first immunization, the most recent immunization, or an intermediate immunization. Plasma can be collected at least 1, at least 2, at least 3, at least 4, at least 5, at least 6, at least 7, at least 8, at least 9, at least 10, at least 15, at least 20, at least 21, at least 22, at least 23, at least 24, at least 25, at least 26 at least 27, at least 28, at least 29, or at least 30 days, or more, after an immunization. In some embodiments, plasma is collected at most 2, at most 3, at most 4, at most 5, at most 6, at most 7, at most 8, at most 9, at most 10, at most 15, at most 20, at most 21, at most 22, at most 23, at most 24, at most 25, at most 26, at most 27, at most 28, at most 29, at most 30, at most at most 35, at most 42, at most 49, or at most 56 days after an immunization. In some embodiments, plasma is collected about 1, 2, 3, 4, 5, 6, 7, 8, 9, 10, 11, 12, 14, 16, 17, 18, 20, 21, 22, 23, 24, 25, 26, 27, 28, 29, 30, 31, 32, 33, 34, 35, 36, 37, 38, 39, 40, 41, or 42 days, or more after an immunization. In some embodiments, a composition described herein can include plasma collected after administration of the immunogenic composition/antigen described herein.

Plasma can be frozen (e.g., stored or transported frozen). In some embodiments, plasma is maintained fresh, or antibodies (e.g., heavy chain antibodies or nanobodies) are purified from fresh plasma.

Nanobodies are purified from plasma using techniques known to those of skill in the art, e.g., by affinity purification. In some cases, chimeric heavy chain antibodies described herein can be purified from plasma using any appropriate technique such as by affinity purification.

In some cases, methods for producing a protein provided herein (e.g., a human nanobody, a humanized nanobody, a fully mouse heavy chain antibody lacking CH1, or a chimeric heavy chain antibody with or without CH1) can involve expression in mammalian cells, although recombinant proteins can also be produced using insect cells, yeast, bacteria, or other cells under the control of appropriate promoters. In some cases, an antibody (e.g., a heavy chain antibody or nanobody) provided herein can be recombinantly produced in prokaryotic hosts such as E. coli, Bacillus brevis, Bacillus subtilis, Bacillus megaterium, Lactobacillus zeae/casei, or Lactobacillus paracasei. In some cases, an antibody (e.g., a heavy chain antibody or nanobody) provided herein can be recombinantly produced in eukaryotic hosts such as yeast (e.g., Pichia pastoris, Saccharomyces cerevisiae, Hansenula polymorpha, Schizosaccharomyces pombe, Schwanniomyces occidentalis, Kluyveromyces lactis, or Yarrowia lipolytica), filamentous fungi of the genera Trichoderma (e.g., T. reesei) and Aspergillus (e.g., A. niger and A. oryzae), protozoa such as Leishmania tarentolae, insect cells, or mammalian cells (e.g., mammalian cell lines such as Chinese hamster ovary (CHO) cells, Per.C6 cells, mouse myeloma NSO cells, baby hamster kidney (BHK) cells, or human embryonic kidney cell line HEK293). See, for example, the Frenzel et al. reference (Front Immunol., 4:217 (2013)). Mammalian expression vectors may comprise nontranscribed elements such as an origin of replication, a suitable promoter and enhancer, and other 5′ or 3′ flanking nontranscribed sequences, and 5′ or 3′ nontranslated sequences such as necessary ribosome binding sites, a polyadenylation site, splice donor and acceptor sites, and termination sequences. DNA sequences derived from the SV40 viral genome, for example, SV40 origin, early promoter, enhancer, splice, and polyadenylation sites may be used to provide the other genetic elements required for expression of a heterologous DNA sequence.

Appropriate cloning and expression vectors for use with bacterial, fungal, yeast, and mammalian cellular hosts are described in Green & Sambrook, Molecular Cloning: A Laboratory Manual (Fourth Edition), Cold Spring Harbor Laboratory Press (2012) and can be used to produce an antibody (e.g., a human nanobody, a humanized nanobody, a fully mouse heavy chain antibody lacking CH1, or a chimeric heavy chain antibody with or without CH1) provided herein.

Various mammalian cell culture systems can be employed to express and manufacture a recombinant protein or antibody (e.g., a human nanobody, a humanized nanobody, a fully mouse heavy chain antibody lacking CH1, or a chimeric heavy chain antibody with or without CH1) provided herein. Examples of mammalian expression systems that can be used include, without limitation, CHO cells, COS cells, HeLA, and BHK cell lines. Processes of host cell culture for production of protein therapeutics that can be used are described in, e.g., Zhou and Kantardjieff (Eds.), Mammalian Cell Cultures for Biologics Manufacturing (Advances in Biochemical Engineering/Biotechnology), Springer (2014). Purification of protein therapeutics is described in Franks, Protein Biotechnology: Isolation, Characterization, and Stabilization, Humana Press (2013); and in Cutler, Protein Purification Protocols (Methods in Molecular Biology), Humana Press (2010). Formulation of protein therapeutics is described in Meyer (Ed.), Therapeutic Protein Drug Products: Practical Approaches to formulation in the Laboratory, Manufacturing, and the Clinic, Woodhead Publishing Series (2012). Compositions described herein may include a vector, such as a viral vector, e.g., a lentiviral vector, adenoviral or adeno-associated virus encoding a recombinant protein. In some embodiments, a vector, e.g., a viral vector, may comprise a nucleic acid encoding a recombinant protein. In some cases, the processes described herein can be designed to comply with the standards defined for Good Manufacturing Practices (GMP) that will involve several quality controls and an adequate infrastructure and separation of activities to avoid cross-contamination. Finally, the compositions may be labeled and distributed worldwide.

In some embodiments, a therapeutic nanobody preparation described herein can be produced by immunizing, with an antigen described herein, a non-human animal having a humanized immune system. In some cases, a therapeutic nanobody preparation described herein can be produced by immunizing an engineered non-human animal described herein with an antigen of interest as described herein.

A non-human animal having a humanized immune system may be an ungulate, for example, a donkey, a goat, a horse, a cow, or a pig; a rodent, e.g., a rabbit, rat, or a mouse. In some embodiments, a non-human animal having a humanized immune system is a cow (bovine). In some embodiments, a non-human animal having a humanized immune system is a chicken. The non-human animal has a humanized immune system, e.g., its immune system comprises a humanized immunoglobulin gene locus, or multiple humanized immunoglobulin gene loci. In some embodiments, the humanized immunoglobulin gene locus comprises a germ line sequence of human immunoglobulin, allowing the non-human animal to produce humanized antibodies (e.g., fully human antibodies). In some embodiments, a non-human animal with a humanized immune system of the disclosure comprises non-human B cells with a humanized immunoglobulin gene locus. The humanized immunoglobulin gene locus undergoes VDJ recombination during B cell development, thereby allowing for generation of B cells with great diversity of antigen binging specificity. Upon immunization with one or more immunogenic compositions described herein, a plurality of B cell clones respond to their respective cognate antigens, leading to the generation of polyclonal antibodies with a plurality of binding specificities.

A non-human animal provided herein can be any type of non-human animal. For example, a non-human animal designed to express chimeric heavy chain antibodies can be an ungulate, for example, a donkey, a goat, a horse, a cow, or a pig; a rodent, e.g., a rabbit, rat, or a mouse. In some embodiments, a non-human animal provided herein (e.g., a non-human animal designed to express chimeric heavy chain antibodies) can be a cow (bovine). In some embodiments, a non-human animal provided herein (e.g., a non-human animal designed to express chimeric heavy chain antibodies) can be a chicken.

In some embodiments, immunizing a non-human animal of the disclosure with one or more immunogenic compositions described herein activates at least 1, at least 2, at least 3, at least 4, at least 5, at least 6, at least 7, at least 8, at least 9, at least 10, at least 11, at least 12, at least 13, at least 14, at least 15, at least 20, at least 25, at least 30, at least 35, at least 40, at least 50, at least 60, at least 70, at least 80, at least 90, or at least 100 non-human B cell clones in the non-human animal. In some embodiments, immunizing a non-human animal of the disclosure with one or more immunogenic compositions described herein leads to production of polyclonal antiserum that comprises antibodies (e.g., chimeric heavy chain antibodies) that specifically bind at least 1, at least 2, at least 3, at least 4, at least 5, at least 6, at least 7, at least 8, at least 9, at least 10, at least 11, at least 12, at least 13, at least 14, at least 15, at least 20, at least 25, at least 30, at least 35, at least 40, at least 50, at least 60, at least 70, at least 80, at least 90, or at least 100 antigens of the immunogenic composition described herein.

Various techniques for modifying the genome of non-human animals (e.g., non-human animals for immunization) can be employed to develop an animal capable of producing antibodies (e.g., humanized antibodies, fully mouse heavy chain antibodies, or chimeric heavy chain antibodies). A non-human animal can be a transgenic animal, for example, a transgenic animal comprising all or a substantial portion of the humanized immunoglobulin gene locus or loci. A non-human animal can be a transchromosomal animal, for example, a non-human animal that comprises a human artificial chromosome or a yeast artificial chromosome.

A humanized immunoglobulin gene locus can be present on a vector, for example, a human artificial chromosome or a yeast artificial chromosome (YAC). A human artificial chromosome (HAC) comprising the humanized immunoglobulin gene locus can be introduced into an animal. A vector (e.g., HAC) can contain the germline repertoire of the human antibody heavy chain genes (from human chromosome 14) and the human antibody light chain genes, for example, one or both of kappa (from human chromosome 2) and lambda (from human chromosome 22). The HAC can be transferred into cells of the non-human animal species, and the transgenic animals can be produced by somatic cell nuclear transfer. The transgenic animals can also be bred to produce non-human animals comprising the humanized immunoglobulin gene locus.

In some embodiments, a humanized immunoglobulin gene locus is integrated into the non-human animal's genome. For example, techniques comprising homologous recombination or homology-directed repair can be employed to modify the animal's genome to introduce the human nucleotide sequences. Tools such as CRISPR/Cas, TALEN, and zinc finger nucleases can be used to target integration.

Methods of generating non-human animals having humanized immune systems (e.g., non-human animals for immunization having humanized immune systems) have been disclosed. For example, a human artificial chromosome can be generated and transferred into a cell that comprises additional genomic modifications of interest (e.g., deletions of endogenous non-human immune system genes), and the cell can be used as a nuclear donor to generate a transgenic non-human animal.

In some embodiments, the humanized immune system comprises one or more human antibody heavy chains, wherein each gene encoding an antibody heavy chain is operably linked to a class switch regulatory element. Operably linked can mean that a first DNA molecule (e.g., heavy chain gene) is joined to a second DNA molecule (e.g., class switch regulatory element), wherein the first and second DNA molecules are arranged so that the first DNA molecule affects the function of the second DNA molecule. The two DNA molecules may or may not be part of a single contiguous DNA molecule and may or may not be adjacent. For example, a promoter is operably linked to a transcribable DNA molecule if the promoter is capable of affecting the transcription or translation of the transcribable DNA molecule.

In some embodiments, the humanized immune system comprises one or more human antibody light chains. In some embodiments, the humanized immune system comprises one or more human antibody surrogate light chains.

In some embodiments, the humanized immune system comprises an amino acid sequence that is derived from the non-human animal, for example, a constant region, such as a heavy chain constant region or a part thereof. In some embodiments, a humanized immune system comprises an IgG (e.g., an IgG1) heavy chain constant region from the non-human animal (for example, an ungulate-derived IgG (e.g., IgG1) heavy chain constant region). In some embodiments, at least one class switch regulatory element of the genes encoding the one or more human antibody heavy chains is replaced with a non-human (e.g., ungulate-derived) class switch regulatory element, for example, to allow antibody class switching when antibodies are raised against antigens and/or epitopes of the disclosure within the non-human animal.

A humanized immunoglobulin gene locus can comprise non-human elements that are incorporated for compatibility with the non-human animal. In some embodiments, a non-human element can be present in a humanized immunoglobulin gene locus to reduce recognition by any remaining elements of the non-human animal's immune system. In some embodiments, an immunoglobulin gene can be partly replaced with an amino acid sequence from the non-human animal. In some embodiments, a non-human regulatory element can be present in a humanized immunoglobulin gene locus to facilitate expression and regulation of the locus within the non-human animal.

A humanized immunoglobulin gene locus can comprise a human DNA sequence. A humanized immunoglobulin gene locus can be codon optimized to facilitate expression of the encompassed genes (e.g., antibody genes) in the non-human animal.

A non-human animal having a humanized immune system (e.g., a non-human animal for immunization having a humanized immune system) can comprise or can lack endogenous non-human immune system components. In some embodiments, a non-human animal with a humanized immune system can lack non-human antibodies (e.g., lack the ability to produce non-human antibodies). A non-human animal with a humanized immune system can lack, for example, one or more non-human immunoglobulin heavy chain genes, one or more non-human immunoglobulin light chain genes, or a combination thereof.

A non-human animal with a humanized immune system (e.g., a non-human animal for immunization having a humanized immune system) can retain, for example, non-human immune cells. A non-human animal with a humanized immune system can retain non-human innate immune system components (e.g., cells, complement, antimicrobial peptides, etc.). In some embodiments, a non-human animal with a humanized immune system can retain non-human T cells. In some embodiments, a non-human animal with a humanized immune system can retain non-human B cells. In some embodiments, a non-human animal with a humanized immune system can retain non-human antigen-presenting cells. In some embodiments, a non-human animal with a humanized immune system can retain non-human antibodies.

In some embodiments, the non-human animal having a humanized immune system (e.g., a non-human animal for immunization having a humanized immune system) comprises any feature or any combination of features or any methods of making as disclosed in U.S.

Patent Application Publication No. 2017/0233459, which is hereby incorporated by reference in its entirety. In some embodiments, the non-human animal having a humanized immune system (e.g., a non-human animal for immunization having a humanized immune system) comprises any feature or any combination of features or any methods of making as disclosed in Kuroiwa et al., Nat. Biotechnol., 27(2):173-81 (2009); Matsushita et al., PLos ONE, 9(3):e90383 (2014); Hooper et al., Sci. Transl. Med., 6(264):264ra162 (2014); Matsushit et al., PLoS ONE, 10(6): e0130699 (2015); Luke et al., Sci. Transl. Med., 8(326):326ra21 (2016); Dye et al., Sci. Rep., 6:24897 (2016); Gardner et al., J. Virol., 91(14) (2017); Stein et al., Antiviral Res., 146:164-173 (2017); Silver, Clin. Infect. Dis., 66(7):1116-1119 (2018); Beigel et al., Lancet Infect. Dis., 18(4):410-418 (2018); Luke et al., J Inf Dis., 218(suppl_5):S636-S648 (2018), each of which is hereby incorporated by reference in its entirety.

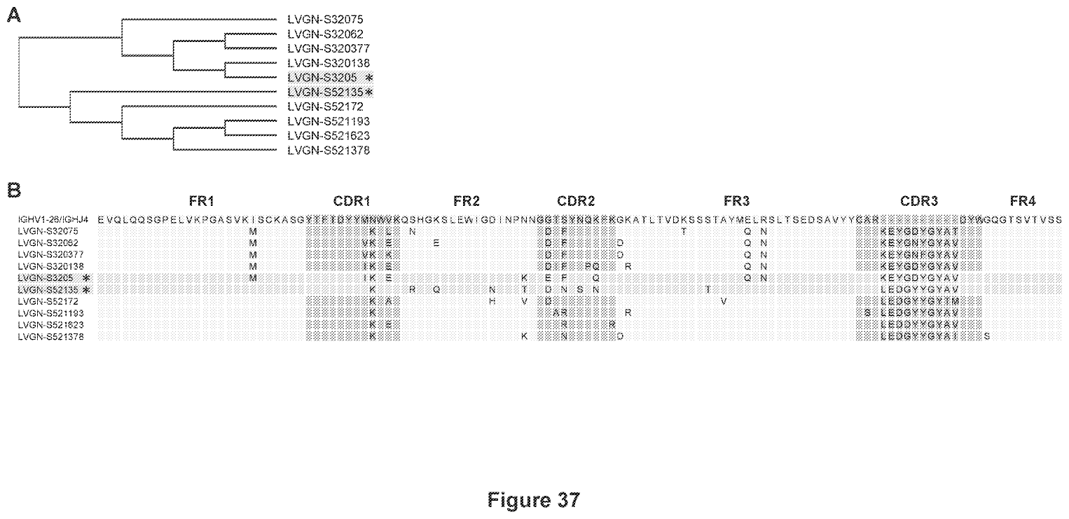

This document also provides antibodies (e.g., nanobodies or heavy chain antibodies) that include the CDRs described herein (e.g., as described in Table 2, FIG. 37, or SEQ ID NOs:1-24). Such antibodies can be configured to be a human antibody, a humanized antibody, or a mouse antibody. In some cases, an antibody (e.g., nanobodies or heavy chain antibodies) provided herein can include the CDRs as described herein (e.g., as described in Table 2, FIG. 37, or SEQ ID NOs:1-24) and can be a monoclonal antibody (e.g., a monoclonal nanobody or monoclonal heavy chain antibody).

In some cases, an antibody (e.g., a nanobody or heavy chain antibody) provided herein can include three CDRs. The first CDR can be selected from the group consisting of SEQ ID NOs:1-7 or SEQ ID NOs:1-7 with one, two, or three amino acid modifications (e.g., additions, deletions, or substitutions). The second CDR can be selected from the group consisting of SEQ ID NOs:8-15 or SEQ ID NOs:8-15 with one, two, or three amino acid modifications (e.g., additions, deletions, or substitutions). The third CDR can be selected from the group consisting of SEQ ID NOs:16-24 or SEQ ID NOs:16-24 with one, two, or three amino acid modifications (e.g., additions, deletions, or substitutions).

TABLE 2
Exemplary CDRs of heavy chain antibodies
having the ability to bind to a SARS-
CoV2 Spike antigen.

| Client | | SEQ | | SEQ | | SEQ |
|---|---|---|---|---|---|---|
| Clone | | ID | | ID | | ID |
| Name | CDR1 | NO | CDR2 | NO | CDR3 | NO |
| LVGN-S32075 | YTFTDYY | 1 | GDTFYNQ | 8 | CARKEYG | 16 |
| | MKWLK | | KFK | | DYGYATD | |
| | | | | | YW | |
| LVGN-S32062 | YTFTDYY | 2 | GDTFYNQ | 8 | CARKEYG | 17 |
| | VKWEK | | KFK | | NYGYAVD | |
| | | | | | YW | |
| LVGN-S320377 | YTFTDYY | 3 | GDTFYNQ | 8 | CARKEYG | 18 |
| | VKWKK | | KFK | | NFGYAVD | |
| | | | | | YW | |
| LVGN-S320138 | YTFTDYY | 4 | GDIFYNP | 9 | CARKEYG | 19 |
| | IKWEK | | QFK | | DYGYAVD | |
| | | | | | YW | |
| LVGN-S3205 | YTFTDYY | 4 | GETFYNQ | 10 | CARKEYG | 19 |
| | IKWEK | | QFK | | DYGYAVD | |
| | | | | | YW | |
| LVGN-S52135 | YTFTDYY | 5 | GDTNYSQ | 11 | CARLEDG | 20 |
| | MKWVK | | NFK | | YYGYAVD | |
| | | | | | YW | |
| LVGN-S52172 | YTFTDYY | 6 | GDTSYNQ | 12 | CARLEDG | 21 |
| | MKWAK | | KFK | | YYGYTMD | |
| | | | | | YW | |
| LVGN-S521193 | YTFTDYY | 5 | GGARYNQ | 13 | CSRLEDG | 22 |
| | MKWVK | | KFK | | YYGYAVD | |
| | | | | | YW | |
| LVGN-S521623 | YTFTDYY | 7 | GGTRYNQ | 14 | CARLEDD | 23 |
| | MKWEK | | KFR | | YYGYAVD | |
| | | | | | YW | |
| LVGN-S521378 | YTFTDYY | 5 | GGTNYNQ | 15 | CARLEDG | 24 |
| | MKWVK | | KFK | | YYGYAID | |
| | | | | | YW | |

In some cases, an antibody (e.g., a nanobody or heavy chain antibody) provided herein can be or can have a heavy chain variable domain having a CDR1 with the amino acid sequence of SEQ ID NO:1, a CDR2 with the amino acid sequence of SEQ ID NO:8, and a CDR3 with the amino acid sequence of SEQ ID NO: 16. In some cases, an antibody (e.g., a nanobody or heavy chain antibody) provided herein can be or can have a heavy chain variable domain having a CDR1 with the amino acid sequence of SEQ ID NO:2, a CDR2 with the amino acid sequence of SEQ ID NO:8, and a CDR3 with the amino acid sequence of SEQ ID NO:17. In some cases, an antibody (e.g., a nanobody or heavy chain antibody) provided herein can be or can have a heavy chain variable domain having a CDR1 with the amino acid sequence of SEQ ID NO:3, a CDR2 with the amino acid sequence of SEQ ID NO:8, and a CDR3 with the amino acid sequence of SEQ ID NO:18. In some cases, an antibody (e.g., a nanobody or heavy chain antibody) provided herein can be or can have a heavy chain variable domain having a CDR1 with the amino acid sequence of SEQ ID NO:4, a CDR2 with the amino acid sequence of SEQ ID NO:9, and a CDR3 with the amino acid sequence of SEQ ID NO:19. In some cases, an antibody (e.g., a nanobody or heavy chain antibody) provided herein can be or can have a heavy chain variable domain having a CDR1 with the amino acid sequence of SEQ ID NO:4, a CDR2 with the amino acid sequence of SEQ ID NO:10, and a CDR3 with the amino acid sequence of SEQ ID NO:19. In some cases, an antibody (e.g., a nanobody or heavy chain antibody) provided herein can be or can have a heavy chain variable domain having a CDR1 with the amino acid sequence of SEQ ID NO:5, a CDR2 with the amino acid sequence of SEQ ID NO: 11, and a CDR3 with the amino acid sequence of SEQ ID NO:20. In some cases, an antibody (e.g., a nanobody or heavy chain antibody) provided herein can be or can have a heavy chain variable domain having a CDR1 with the amino acid sequence of SEQ ID NO:6, a CDR2 with the amino acid sequence of SEQ ID NO:12, and a CDR3 with the amino acid sequence of SEQ ID NO:21. In some cases, an antibody (e.g., a nanobody or heavy chain antibody) provided herein can be or can have a heavy chain variable domain having a CDR1 with the amino acid sequence of SEQ ID NO:5, a CDR2 with the amino acid sequence of SEQ ID NO:13, and a CDR3 with the amino acid sequence of SEQ ID NO:22. In some cases, an antibody (e.g., a nanobody or heavy chain antibody) provided herein can be or can have a heavy chain variable domain having a CDR1 with the amino acid sequence of SEQ ID NO:7, a CDR2 with the amino acid sequence of SEQ ID NO:14, and a CDR3 with the amino acid sequence of SEQ ID NO:23. In some cases, an antibody (e.g., a nanobody or heavy chain antibody) provided herein can be or can have a heavy chain variable domain having a CDR1 with the amino acid sequence of SEQ ID NO:5, a CDR2 with the amino acid sequence of SEQ ID NO:15, and a CDR3 with the amino acid sequence of SEQ ID NO:24.

In some cases, a CDR3 shown in Table 2 can lack the first C residue and can lack the last W residue.

As indicated herein, the amino acid sequences described herein can include amino acid modifications (e.g., the articulated number of amino acid modifications). Such amino acid modifications can include, without limitation, amino acid substitutions, amino acid deletions, amino acid additions, and combinations thereof. In some cases, an amino acid modification can be made to improve the binding and/or contact with an antigen and/or to improve a functional activity of an antibody (e.g., a nanobody or a heavy chain antibody) provided herein. In some cases, an amino acid substitution within an articulated sequence identifier can be a conservative amino acid substitution. For example, conservative amino acid substitutions can be made by substituting one amino acid residue for another amino acid residue having a similar side chain. Families of amino acid residues having similar side chains can include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine).

In some cases, an amino acid substitution within an articulated sequence identifier can be a non-conservative amino acid substitution. Non-conservative amino acid substitutions can be made by substituting one amino acid residue for another amino acid residue having a dissimilar side chain. Examples of non-conservative substitutions include, without limitation, substituting (a) a hydrophilic residue (e.g., serine or threonine) for a hydrophobic residue (e.g., leucine, isoleucine, phenylalanine, valine, or alanine); (b) a cysteine or proline for any other residue; (c) a residue having a basic side chain (e.g., lysine, arginine, or histidine) for a residue having an acidic side chain (e.g., aspartic acid or glutamic acid); and (d) aresidue having a bulky side chain (e.g., phenylalanine) for glycine or other residue having a small side chain.

Methods for generating an amino acid sequence variant (e.g., an amino acid sequence that includes one or more modifications with respect to an articulated sequence identifier) can include site-specific mutagenesis or random mutagenesis (e.g., by PCR) of a nucleic acid encoding the antibody or fragment thereof. See, for example, Zoller, Curr. Opin. Biotechnol. 3: 348-354 (1992). Both naturally occurring and non-naturally occurring amino acids (e.g., artificially-derivatized amino acids) can be used to generate an amino acid sequence variant provided herein.

This document also provides pharmaceutical compositions or pharmaceutical formulations that can include any of the antibodies (e.g., nanobodies or heavy chain antibodies) provided herein. Any of the pharmaceutical compositions or pharmaceutical formulations may also include additional cells or cellular components.

As described herein, an antigen can be administered to a non-human animal provided herein to produce antibodies (e.g., heavy chain antibodies such as chimeric heavy chain antibodies). In some embodiments, the antigen is an endogenous antigen or self-antigen to the subject (e.g., the mammal, for example, a human, cow, horse, non-human primate, rabbit, goat, sheep, dog, pig, mouse, rat).

In some embodiments, the antigen is a lipid. In some embodiments, the lipid is a membrane lipid and soluble lipid. Examples of membrane lipids include, but is not limited to, diacylglycerol (DAG), phosphatidic acid (PA), phosphatidylserine (PS), phosphatidylinositol (PtdIns), phosphatidylethanolamine (PE), phosphatidylcholine (PtC), phosphatidylglycerol (PG), sphingomyelin, phosphorylcholine (PC), and cardiolipin.

Examples of soluble lipids include, but is not limited to, low-density lipoprotein (LDL), malondialdehyde-LDL (MDA-LDL), oxidized LDL (oxLDL), advanced glycation end products-LDL (AGE-LDL), MDA, and lysophosphatidylcholine (LPC).

In some embodiments, the antigen is associated with an immune cell (e.g., the antigen is a cell surface protein on an immune cell). Examples of immune cells include, but is not limited to, peripheral blood mononuclear cells (PBMCs), macrophages, T cells, dendritic cells, neutrophils, and monocytes.

In some embodiments, the antigen is a peptide, protein, lipid, molecule, or other biological compound that binds with an immune cell.

In some embodiments, the antigen is associated with an injured cell, a dead cell, or a dying cell. Cell injury and/or death can be caused by any underlying pathology, such as apoptosis, necrosis, ischemia, etc.

In some embodiments, the antigen is another immunoglobin, such as, for example IgG.

In some embodiments, the antigen can be an antigen listed in Table 3.

TABLE 3
Examples of antigens that can be used to immunize a non-human animal provided herein.

| | | | | | | # of | | Average |
|---|---|---|---|---|---|---|---|---|
| | | | | | | Clonotypes | # of | CDR3 |
| | | Sample | Total Raw | Successfully | Overlapped | with >=5 | IGHVs | length |
| Antigen | Genotype | ID | Reads | Aligned | and Aligned | reads | mapped | (# a.a.) |
| SARS-CoV2 | WT | 342 | 2,291,736 | 2,017,892 | 1,643,418 | 1,316 | 79 | 13.8 |
| Spike | | | | | | | | |
| SARS-CoV2 | WT | 345 | 2,148,017 | 1,828,914 | 1,516,225 | 2,611 | 99 | 13.3 |
| Spike | | | | | | | | |
| SARS-CoV2 | SM | 263 | 2,330,741 | 2,051,048 | 1,794,151 | 8,097 | 103 | 13.3 |
| Spike | | | | | | | | |
| SARS-CoV2 | SM | 320 | 2,503,240 | 2,040,036 | 1,794,759 | 11,565 | 108 | 13.6 |
| Spike | | | | | | | | |
| SARS-CoV2 | SM | 521 | 2,500,000 | 1,045,815 | 903,252 | 30,242 | 109 | 13.7 |
| Spike | | | | | | | | |
| PD-L1 | WT | 718-1 | 1,672,139 | 1,474,961 | 1,328,428 | 2,063 | 90 | 14.3 |
| PD-L1 | WT | 718-2 | 1,252,533 | 1,147,322 | 1,035,530 | 1,151 | 87 | 14.4 |
| PD-L1 | SM | 719-1 | 1,911,053 | 1,506,500 | 1,403,299 | 17,864 | 113 | 13.9 |
| PD-L1 | SM | 719-2 | 2,593,888 | 1,944,486 | 1,880,851 | 12,516 | 111 | 13.9 |
| Rabbit IgG | WT | 988 | 1,260,250 | 699,909 | 641,341 | 2,011 | 94 | 14.0 |
| Rabbit IgG | SM | 989 | 4,045,040 | 2,095,584 | 2,019,297 | 47,959 | 121 | 14.0 |
| Rabbit IgG | SM | 990 | 3,964,810 | 2,121,189 | 2,049,181 | 59,718 | 121 | 13.9 |
| Rabbit IgG | SM | 991 | 4,367,973 | 2,217,479 | 2,145,265 | 45,563 | 123 | 13.9 |
| Rat IgG | SM | 992 | 1,789,898 | 845,743 | 809,492 | 30,691 | 118 | 13.8 |
| Rat IgG | SM | 993 | 2,338,614 | 1,076,919 | 1,038,454 | 40,024 | 122 | 14.0 |
| Rat IgG | SM | 1024 | 2,027,019 | 1,031,155 | 983,144 | 37,096 | 120 | 14.0 |
| Goat IgG | SM | 1043 | 1,338,913 | 1,017,750 | 971,713 | 8,999 | 105 | 14.1 |
| Goat IgG | SM | 1044 | 1,448,252 | 1,031,304 | 987,794 | 6,276 | 111 | 14.1 |

This document also provides methods for treating or preventing a disease or disorder. In some embodiments, an antibody (e.g., a nanobody or heavy chain antibody) provided herein can be used for the treatment or prevention of a disease or a disorder. For example, the disease or disorder can be an inflammatory disease, an autoimmune disease, a cardiovascular disease, or a neurodegenerative disease. In some embodiments, the disease or disorder can be diabetes, systemic lupus erythematosus, multiple sclerosis, rheumatoid arthritis, scleroderma, Crohn's disease, ulcerative colitis, mixed connective tissue disease, Sjogren syndrome, or polymyositis, dermatomyositis.

In some cases, a composition comprising an antibody (e.g., a nanobody or heavy chain antibody) provided herein can be intended to be used in the prophylaxis and/or treatment of a disease or disorder (e.g., an autoimmune disease or an inflammatory disorder). Accordingly, this document further provides a pharmaceutical formulation, which comprises an antibody (e.g., a nanobody or heavy chain antibody) provided herein and a pharmaceutically acceptable carrier therefor. The pharmaceutical formulations may be prepared by conventional techniques, e.g. as described in Remington: The Science and Practice of Pharmacy 2005, Lippincott, Williams & Wilkins.

The pharmaceutically acceptable carriers can be either solid or liquid. Solid form preparations include powders, tablets, pills, capsules, cachets, suppositories, and dispersible granules. A solid carrier can be one or more excipients which may also act as diluents, flavoring agents, solubilizers, lubricants, suspending agents, binders, preservatives, wetting agents, tablet disintegrating agents, or an encapsulating material.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like, as, for example, described elsewhere (Gervasi et al., Eur. J. Pharmaceutics and Biopharmaceutics, 131:8-24 (2018)).

Examples of pharmaceutically acceptable carriers that can be used to make a pharmaceutical composition provided herein include, without limitation, water, lactic acid, citric acid, sodium chloride, sodium citrate, sodium succinate, sodium phosphate, a surfactant (e.g., polysorbate 20, polysorbate 80, or poloxamer 188), dextran 40, or a sugar (e.g., sorbitol, mannitol, sucrose, dextrose, or trehalose), and combinations thereof.

Other ingredients that can be included within a pharmaceutical composition provided herein include, without limitation, amino acids such as glycine or arginine, antioxidants such as ascorbic acid, methionine, or ethylenediaminetetraacetic acid (EDTA), anticancer agents such as enzalutamide, imanitib, gefitinib, erlotini, sunitinib, lapatinib, nilotinib, sorafenib, temsirolimus, everolimus, pazopanib, crizotinib, ruxolitinib, axitinib, bosutinib, cabozantinib, ponatinib, regorafenib, ibrutinib, trametinib, perifosine, bortezomib, carfilzomib, batimastat, ganetespib, obatoclax, navitoclax, taxol, paclitaxel, or bevacizumab, and combinations thereof.

In some cases, an antibody (e.g., a nanobody or heavy chain antibody) provided herein can be formulated for parenteral administration and may be presented in unit dose form in ampoules, pre-filled syringes, small volume infusion or in multi-dose containers, optionally with an added preservative. The compositions may take such forms as suspensions, solutions, or emulsions in oily or aqueous vehicles, for example solutions in aqueous polyethylene glycol. Examples of oily or non-aqueous carriers, diluents, solvents or vehicles include propylene glycol, polyethylene glycol, vegetable oils, and injectable organic esters, and may contain agents such as preserving, wetting, emulsifying or suspending, stabilizing and/or dispersing agents. In some cases, the formulation can comprise about 0.5% to 75% by weight of the active ingredient(s) with the remainder consisting of suitable pharmaceutical excipients as described herein.

The compositions provided herein can be administered concurrently, simultaneously, or together with a pharmaceutically acceptable carrier or diluent, especially and preferably in the form of a pharmaceutical composition thereof, whether by oral, rectal, or parenteral (including subcutaneous) route, in an effective amount.

This document also provides pharmaceutical compositions comprising B cells (e.g., B cells isolated from a non-human animal provided herein), monoclonal antibodies (e.g., a monoclonal heavy chain antibody or a monoclonal nanobody), and/or polyclonal antibodies (e.g., a polyclonal heavy chain antibody or a polyclonal nanobody). Such pharmaceutical compositions may comprise an adjuvant, a buffer, salts or a combination hereof.

An adjuvant is a pharmacological and/or immunological agent that modifies the effect of other agents. In some embodiments, adjuvants may be added to the compositions to modify the immune response by boosting it such as to give a higher amount of antibodies and/or a longer lasting protection, thus minimizing the amount of injected antigenic material. Adjuvants may also be used to enhance the efficacy of a composition by helping to subvert the immune response to particular cell types of the immune system, for example by activating the T cells instead of antibody-secreting B cells dependent on the type of the composition. In one embodiment, the composition can comprise at least one adjuvant. In another embodiment, the adjuvant can be aluminum based. Aluminum adjuvants may be aluminum phosphate, aluminum hydroxide, amorphous aluminum hydroxyphosphate sulfate and/or a combination hereof. Other adjuvants may be included as well.

In another embodiment, a composition described herein can comprise at least one buffer. In one embodiment, the buffer can be PBS and/or histidine based. In another embodiment, the buffer can have a pH between 6.0 and 7.5. In an embodiment, the buffer can be isotonic such as a buffer of 0.6%-1.8% NaCl.

An emulsifier (also known as an “emulgent”) is a substance that stabilizes an emulsion by increasing its kinetic stability. One class of emulsifiers is known as “surface active agents,” or surfactants. Polysorbates are a class of emulsifiers used in some pharmaceuticals and food preparation. Common brand names for polysorbates include Alkest, Canarcel, and Tween. Some examples of polysorbates are Polysorbate 20, Polysorbate 40, Polysorbate 60, Polysorbate 80. In one embodiment, a composition provided herein can comprise an emulsifier such as one of the above described polysorbates. In one embodiment, the composition can comprise 0.001-0.02% polysorbate 80. Other polysorbates or emulsifiers may be used as described herein as well.

In some cases, a pharmaceutical composition provided herein can comprise an antibody (e.g., a nanobody or heavy chain antibody) provided herein and a pharmaceutically acceptable carrier. In some cases, a pharmaceutical composition provided herein can comprise an antibody (e.g., a nanobody or heavy chain antibody) provided herein, a pharmaceutically acceptable carrier, and a buffer. In some cases, a pharmaceutical composition provided herein can comprise an antibody (e.g., a nanobody or heavy chain antibody) provided herein, a pharmaceutically acceptable carrier, and an emulsifier. In some cases, a pharmaceutical composition provided herein can comprise an antibody (e.g., a nanobody or heavy chain antibody) provided herein, a pharmaceutically acceptable carrier, and an adjuvant. In some cases, a pharmaceutical composition provided herein can comprise an antibody (e.g., a nanobody or heavy chain antibody) provided herein, a pharmaceutically acceptable carrier, a buffer, and an adjuvant. In some cases, a pharmaceutical composition provided herein can comprise an antibody (e.g., a nanobody or heavy chain antibody) provided herein, a pharmaceutically acceptable carrier, an emulsifier, and an adjuvant. In some cases, a pharmaceutical composition provided herein can comprise an antibody (e.g., a nanobody or heavy chain antibody) provided herein, a pharmaceutically acceptable carrier, an emulsifier, a buffer, and an adjuvant.

Any appropriate methods can be used to design and construct the nucleic acid and polypeptide agents described herein. Generally, recombinant methods can be used. See, in general, Smales & James (Eds.), Therapeutic Proteins: Methods and Protocols (Methods in Molecular Biology), Humana Press (2005); and Crommelin, Sindelar & Meibohm (Eds.), Pharmaceutical Biotechnology: Fundamentals and Applications, Springer (2013). Methods of designing, preparing, evaluating, purifying and manipulating nucleic acid compositions are described in Green and Sambrook (Eds.), Molecular Cloning: A Laboratory Manual (Fourth Edition), Cold Spring Harbor Laboratory Press (2012).

This document also provides methods for administering a composition (e.g., a pharmaceutical composition provided herein) containing an antibody (e.g., a heavy chain antibody or nanobody) provided herein to a mammal (e.g., a human) to treat a disease or disorder. For example, a composition (e.g., a pharmaceutical composition provided herein) containing one or more antibodies provided herein can be administered to a mammal (e.g., a human) having an inflammatory disease to treat that mammal. In some cases, a composition (e.g., a pharmaceutical composition provided herein) containing one or more antibodies provided herein can be administered to a mammal (e.g. a human) to reduce severity of an inflammatory disease within the mammal and/or to increase the survival of the mammal suffering from an inflammatory disease compared to a mammal (e.g. a human) who was not administered the composition.

The compositions described herein can be administered to a subject by any mode of delivery, including, for example, by parenteral injection (e.g. subcutaneously, intraperitoneally, intravenously, intramuscularly, or to the interstitial space of a tissue), or by rectal, oral (e.g. tablet, spray), vaginal, topical, transdermal (see, e.g., International PCT Patent Application Publication No. WO99/27961) or transcutaneous (see, e.g., International PCT Patent Application Publication No. WO02/074244 and International PCT Patent Application Publication No. WO02/064162), intranasal (see, e.g., International PCT Patent Application Publication No. WO03/028760), ocular, aural, pulmonary or other mucosal administration. Multiple doses can be administered by the same or different routes.

The compositions (e.g., a composition containing a heavy chain antibody and/or nanobody produced from a non-human animal provided herein) provided herein can be administered prior to, concurrent with, or subsequent to delivery of other therapeutics. Also, the site of administration may be the same or different as other therapeutics that are being administered.

Dosage treatment with a composition provided herein may be a single dose schedule or a multiple dose schedule. A multiple dose schedule is one in which a primary course of vaccination may be with 1-10 separate doses, followed by other doses given at subsequent time intervals, chosen to maintain and/or reinforce the immune response, for example at 1-4 months for a second dose, and if needed, a subsequent dose(s) after several months. The dosage regimen will also, at least in part, be determined by the potency of the modality, the delivery employed, the need of the subject and be dependent on the judgment of the practitioner.

The above described embodiments can be combined to achieve the aforementioned functional characteristics. This is also illustrated by the below examples which set forth exemplary combinations and functional characteristics achieved.

### EXAMPLES

### Example 1—Genetic Engineering of Singularity mice

### Genetic Engineering Procedures

The LVGN-YF ES cell line (40, XY) was established using blastocysts isolated from crosses of 129S6 and C57BL/6N mice and was used for all genetic engineering. This F1 hybrid ES cell line exhibited robust germline competence after multiple rounds of genetic modifications, and the use of a F1 hybrid ES cell line also allowed for use of strain-specific SNPs to identify sequential genetic modifications that occurred on the same chromosome. ES cells were cultured in Knockout DMEM supplemented with 20% ES cell-qualified fetal bovine serum (FBS), 0.1 mM MEM non-essential amino acids, 0.1 mM 2-mercaptoethanol, 1 mM sodium pyruvate, 2 mM GlutaMAX-I supplement, 100 units/mL penicillin-streptomycin, 25 nM MEK inhibitor PD98059 (Sigma), 3 nM GSK-3 inhibitor CH1R99021 (Sigma), and 1,000 units/mL mouse leukemia inhibitory factor (LIF, Sigma) on feeder cells. The hygro^{R}/neo^{R}/puro^{R} triple-resistant feeder cell line LVGN-SHNPL was engineered from the SNL76/7 feeder cell line and was maintained in Knockout DMEM supplemented with 10% ES cell-qualified FBS, 0.1 mM MEM non-essential amino acids, 0.1 mM 2-mercaptoethanol, 1 mM sodium pyruvate, 2 mM GlutaMAX-I supplement, and 100 units/mL penicillin-streptomycin. Feeder cells were prepared by treating the proliferating cells for 3 hours with 10 mg/mL Mitomycin C (Sigma).

All transfections were done either with lipofection using Lipofectamine LTX (ThermoFisher), or with electroporation using a Bio-Rad Gene Pulser II apparatus. For lipofection, 0.1-1×10⁶ dissociated ES cells were mixed with 0.5-2.5 μg plasmid DNA-lipofectamine complex in Opti-MEM following instructions provided by the manufacturer and cultured overnight in growth medium, and antibiotic selection was applied 24 hours later as needed. For electroporation, 0.5-1.5×10⁷ ES cells were mixed with 10-30 μg plasmid and/or BAC DNA in PBS and electroporated at 250V/500 μF in a 4-mm gap cuvette and cultured overnight in growth medium, and antibiotic selection was applied 24 hours later as needed. The antibiotic concentrations used for selection were 250 μg/mL for Geneticin (G418), 200 μg/mL for Hygromycin B, and 5 μg/mL for Puromycin. For CRISPR-mediated gene editing, Cas9 and sgRNAs were delivered as separate plasmids, which were co-transfected with PGK-puro or PGK-hygro genes and selected for 2 days with the corresponding antibiotics to enrich for transfectants, or were co-transfected with the HDR template and selected for 10-14 days with the corresponding antibiotics to derive stably transfected clones. For removing selection marker cassettes flanked with recombination sites (lox-lox, frt-frt, or attB-attP), ES cells were transiently transfected with plasmids expressing the corresponding recombinase or integrase (Cre, Flp, or ϕC31, respectively) and plated at low density to isolate individual clones. For recombinase-mediated cassette exchange (RMCE), ES cells were co-transfected with the RMCE construct and a Cre expressing plasmid and selected with the corresponding antibiotics for 10-14 days. ES cell colonies were picked into 96-well plates for expansion and genotyping by PCR to screen for the desired mutations followed by Sanger sequencing. Sequence verified positive clones were expanded from 96-well to 24-well then to 6-well plates and cryopreserved.

Positive ES cell lines carrying the engineered mutations were used to produce chimeras following standard procedures. Briefly, blastocysts were isolated from superovulated C57BL/6N females at 3.5 dpc, microinjected with ES cells, and then transferred into the uterus of 2.5 dpc pseudopregnant Swiss Webster females for implantation. High-percentage chimeric males were mated with C57BL/6N females for germline transmission of the engineered mutations. Heterozygous F1 mice were identified with junctional PCR from genomic DNA isolated from biopsies. F1 mice were then intercrossed to generate F2 mice homozygous for the same mutation or crossed to other lines as needed. All engineered mice were maintained in a mixed 129S6 and C57BL/6N

### BACKGROUND

Generation of the Singularity Musculus allele

Igh is one of the largest loci in the mouse genome, spanning several megabases (Mb) near the right end of chromosome 12 q arm. It encodes many disparate elements involved in the generation of virtually endless diversity of antibodies. The locus contains a variable region of more than 2.5 Mb that encodes hundreds of gene segments responsible for much of the antibody diversity and a much smaller 220 kb constant CH region encoding expression for several antibody classes and subtypes (FIG. 1A). The CH region has eight CH genes that encode different Ig isotypes: Cμ (Ighm), Cδ (Ighd), Cγ3 (Ighg3), Cγ1(Ighg1), Cγ2b (Ighg2b), Cγ2a/2c (Ighg2a/2c), Cε (Ighe), and Cα (Igha). Regulatory elements flanking as well as situated throughout this region are involved in class switch recombination (CSR) and timely expression of the isotypes (FIG. 2).

In addition to the above elements, located upstream of each Ig isotype (except for IgD) are the I promoter/exons and S switch regions. The latter is involved in CSR, and the former is involved in germline transcription of its corresponding Ig isotype. Transcription of Ig isotype is highly regulated. In resting B cells, germline transcription (GLT) is restricted to that of Cμ, driven by the Ep enhancer and constitutive Iμ promoter. In activated B cells in response to antigen encounter and cytokine, transcription of the downstream Ig isotype is activated at its I promoter containing the respective response elements. Simultaneous transcription at the Iμ promoter and the downstream I promoter of the activated Ig isotype results in AID-mediated CSR.

A step-by-step process was used to generate a Singularity musculus allele (FIG. 1B). Unlike the normal tetrameric antibodies produced by wildtype (WT) mice (FIG. 1C), mice homozygous for this allele produce only HCAbs (FIG. 1D), the genetic composition and diversity of which derive entirely from the natural immune repertoire of mice.

Generation of the Singularity musculus allele was accomplished via 3 rounds of genetic engineering in LVGN-YF ES cells (FIG. 3). The Igh locus of these ES cells encoded Cγ2c similar to that found in C57BL/6N mice rather than Cγ2a in the BALB/c strain (FIG. 3A). The first round of modification was performed via CRISPR-mediated non-homologous end joining (NHEJ), which deleted a 92.6 Kb genome DNA fragment spanning Cμ, Cδ, Cγ3, and the first exon of Cγ1 (which encodes the CH1 domain of IgG1) (FIG. 3B). The sgRNAs were designed to cut immediately downstream of the Cμ switch region (Sμ) and upstream of the second exon of Cγ1 (which encodes the hinge domain of IgG1), thereby placing the truncated Cγ1 gene under the direct control of Iμ promoter and Sμ, rendering the otherwise cytokine-inducible transcription of IgG1 to become constitutive, as for the case of IgM in WT allele. The second round of modification was performed via CRISPR-mediated homology directed repair (HDR), which removed a 63.2 Kb genomic DNA fragment spanning Cγ2b, Cγ2c, Cε, and the first three exons of Cα, while introducing a selection marker cassette (PGK/Em7-neo) flanked with frt sites (FIG. 3C). CRISPR cut sites were selected to avoid removal of the 3′γ1E element downstream of Cγ1 and the 5′hsR1 element within intron 3 of Ca. The third round of engineering utilized transient expression of Flp recombinase, which removed the selection marker cassette, leaving a single frt site to be used for synteny verification of later modifications (FIG. 3D). Regulatory elements (including Eμ, Iμ, Sμ, 3′γ1E, 5′hsR1, 3′RR, and 3′CBE) were kept intact to allow high-level constitutive transcription of CH1-truncated IgG1 (IgG1ΔCH1) from the endogenous Igh allele (FIGS. 2 and 3E). The resulting Singularity Musculus mice thus only produce HCAbs of the IgG1 subtype; all other antibody classes (IgM, IgD, IgE, and IgA) and IgG subtypes (IgG2b, IgG2c, and IgG3) were eliminated to avoid any potential mechanisms compromising HCAb production and to facilitate nanobody discovery, expression, and purification.

### Generation of the Singularity HyperDock Allele

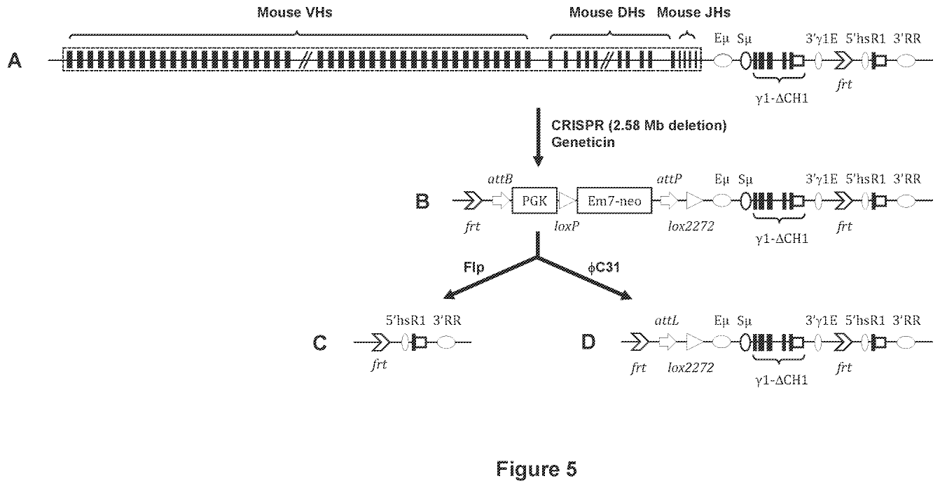

To expand the versatility of the Singularity platform and generate HCAbs from other species, the Singularity HyperDock allele was generated by deleting a 2.58 Mb genomic DNA fragment containing all mouse VH, DH, and J_{H} genes (from upstream of Ighv86-1 to downstream of Ighj4) and inserting a docking cassette for sequential RMCE upstream of Eμ via CRISPR-mediated HDR (FIGS. 4 and 5). The HDR template contained the left homology arm, an frt site, an attB site, a PGK promoter, a loxP site, an Em7-neo cassette, an attP site, and a lox2272 site followed by the right homology arm. The frt site was incorporated to verify the introduced RMCE docking cassette was on the same chromosome (C57BL/6N) as the Singularity Musculus allele upon expression of Flp recombinase. The wild type loxP site, instead of other heterospecific lox sites, was selected to place between the PGK promoter and the Em7-neo cassette to enable highly efficient RMCE events via selection marker swapping. These modifications resulted in a mouse VDJ-null Singularity HyperDock allele that contained RMCE docking sites for sequential introduction of BACs, cloned constructs, or synthetic fragments containing any combinatorial segments of V, D, or J genes of the heavy or light chain alleles from humans or other species (FIG. 5).

Generation of the Singularity Sapiens allelic Series

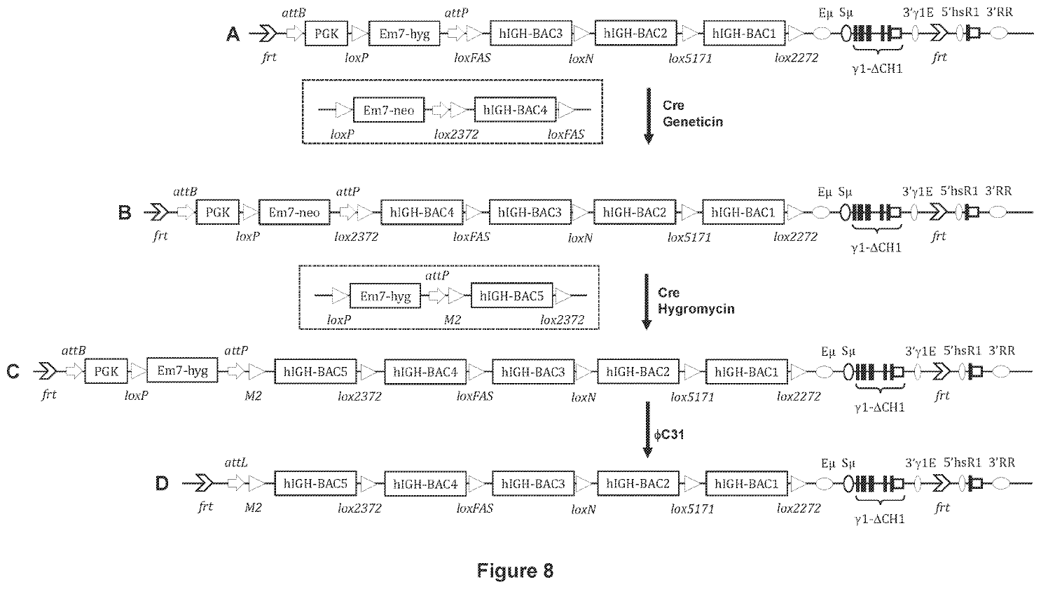

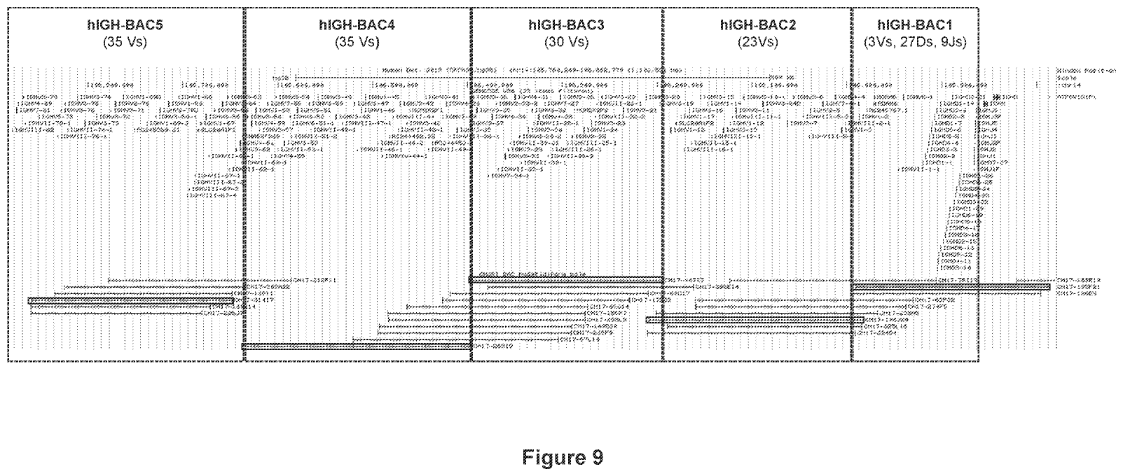

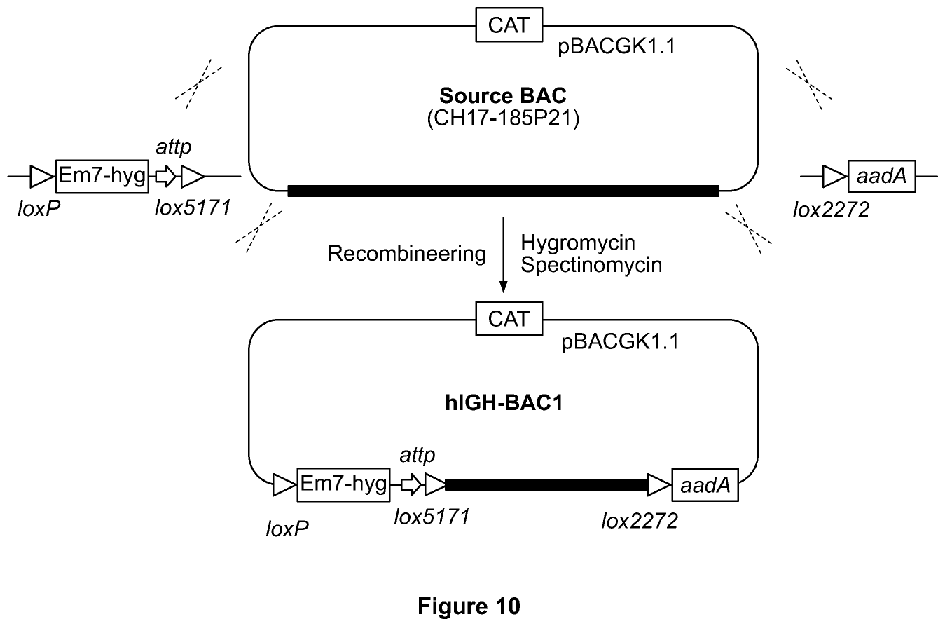

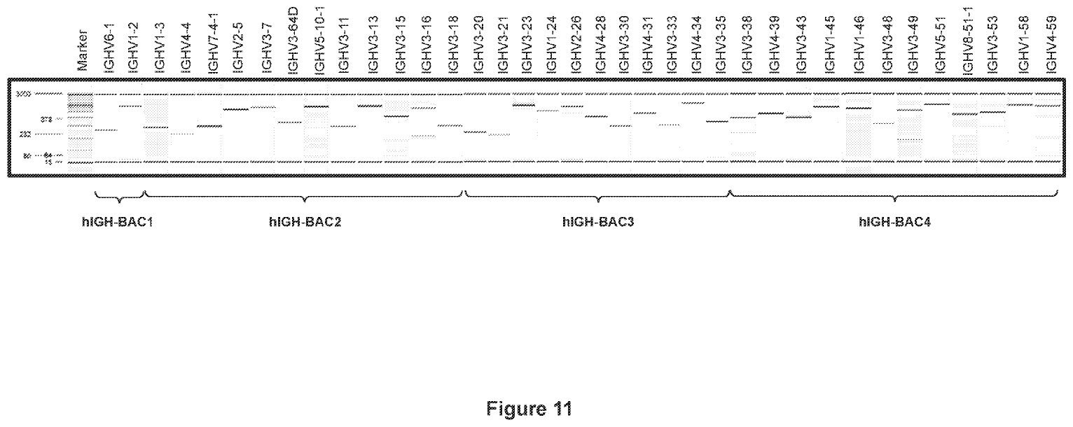

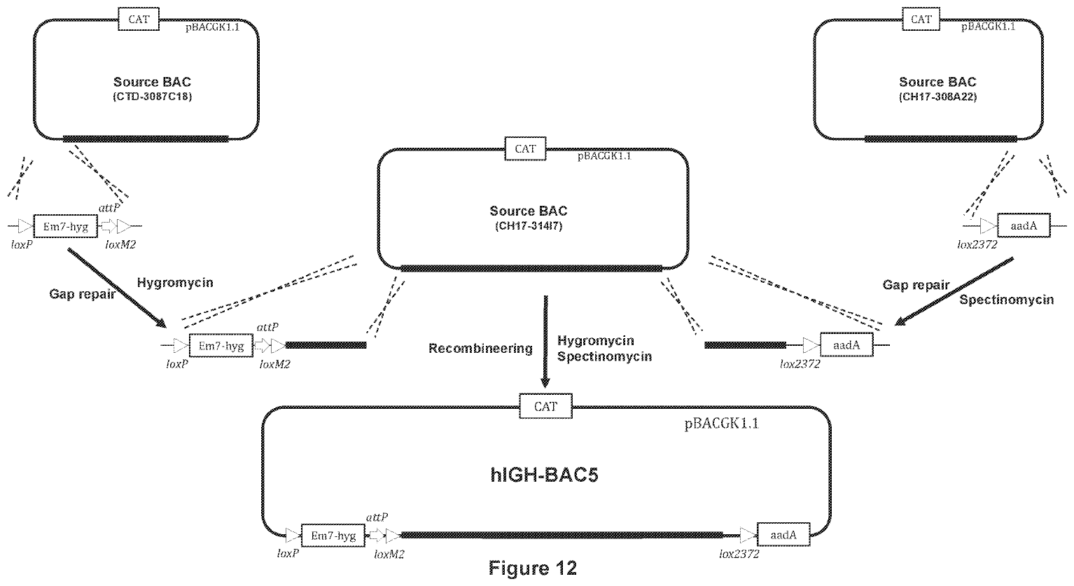

Engineered BACs containing human VH, DH, and J_{H} genes were introduced into the Singularity HyperDock allele to generate the Singularity Sapiens allelic series (FIGS. 6-8). Overlapping IGH BAC clones from the CH17 BAC library and RPC1-11 library (BACPAC resources) (FIG. 9 and Table 4) were modified at both ends by bacterial homologous recombination (recombineering) to incorporate either the Em7-hyg or the Em7-neo cassettes to allow selection marker swapping, while introducing the corresponding heterospecific lox sites (Table 1) flanking the genomic fragment for sequential RMCE. Briefly, a synthetic gBlock (IDT or Twist) containing two 75-150 base pairs (bp) homology arms flanking proper lox site(s) and antibiotic resistance cassette was electroporated into an E. coli strain containing a heat inducible Red recombinase in an electroporation cuvette with 1-mm gap using a Bio-Rad GenePulser II apparatus at 1.75 kV, 25 μF, and 200 ohms. Next, 1.0 mL SOC medium was added to each cuvette and then transferred into a microfuge tube before incubating at 32° C. for 1 hour with shaking (200 rpm). Cells were subsequently plated onto LB agar plate with the corresponding antibiotics. The resulting colonies were screened by PCR with junctional primers followed by Sanger sequencing for verification. The recombineering process is illustrated in FIG. 10 for the first introduced BAC (hIGH-BAC1), which contained three human VH genes (two functional; IGHV1-2 and IGHV6-1), 27 human D_{H} genes, and 9 human J_{H} genes. A loxP-Em7-hyg-attP-lox5171 cassette was introduced via recombineering immediately upstream of the human IGHV1-2 gene at the 5′ end of the original BAC clone, followed by the introduction of a lox2272-aadA cassette immediately downstream of the human IGHJ6 gene at the 3′ end. The engineered BAC was then used for the first round of RMCE (between loxP and lox2272) to introduce the three human VH genes, all human DH genes, and all human J_{H} genes immediately upstream of the mouse Igh intronic enhancer Eμ, while introducing a different heterospecific lox site (lox5171) for the next round of RMCE (FIGS. 7A-7B). Subsequent overlapping BACs were modified in a similar fashion albeit using alternating selection markers (Em7-neo and Em7-hyg) and different heterospecific lox sites, with overlapping fragments trimmed off to assemble the human VDJ genomic region in a stepwise fashion (FIGS. 7C-7D and 8). The original BAC clones and heterospecific lox sites used to reconstruct the complete human VDJ region can be found in Table 4 and Table 1, respectively. The wildtype loxP site, which exhibits high recombination efficiency, was used for each round of RMCE to pair with different heterospecific lox sites. All engineered BACs were confirmed by PacBio SMRT sequencing prior to transfection into ES cells. No mutation of significance was found except for several SNPs and small indels in the intergenic regions. The sequential RMCE process resulted in the generation of a series of humanized singularity alleles with increasing VH diversity (SSV1-5). PCR analysis followed by Sanger sequencing of SSV4 mice with VH-specific primers confirmed integration of all 37 functional VH elements (FIG. 11). The hIGH-BACS was engineered to contain sequences from three source BACs (FIG. 12) and is introduced into SSV4 ES via RMCE to complete the construction of SSV5, which was designed to contain the complete human VH repertoire (126 VHs, 27 DHs, and 9 JHs genes).

TABLE 4
The human genome coordinates (GRCh38/hg38) of the engineered and source BACs
used in the engineering of the Singularity Sapiens allelic series SSV1-SSV5.

| Engineered BAC | Engineered BAC Coordinates | Source BAC | Source BAC Coordinates |
|---|---|---|---|
| hIGH-BAC1 | chr14: 105862806-105989907 | CH17-185P21 | chr14: 105786489-105990780 |
| hIGH-BAC2 | chr14: 105989908-106196572 | CH17-108J24 | chr14: 105982809-106209070 |
| hIGH-BAC3 | chr14: 106196573-106395796 | CH17-447I7 | chr14: 106194147-106395690 |
| hIGH-BAC4 | chr14: 106395797-106633503 | CH17-268I9 | chr14: 106395814-106634186 |
| hIGH-BAC5 | chr14: 106634307-106875815 | CH17-308A22 | chr14: 106634435-106824828 |
| | | CH17-314I7 | chr14: 106648191-106860132 |
| | | CTD-3087C18 | chr14: 106849834-106875815 |

### Generation of Igk and Igl Knockouts for Producing Light-Chain Free Singularity Mice

To prevent unwarranted interference of light chains with the generation of HCAbs in the Singularity mice, the murine light chains were removed by deleting the V and J gene segments from the Igκ and IgL loci.

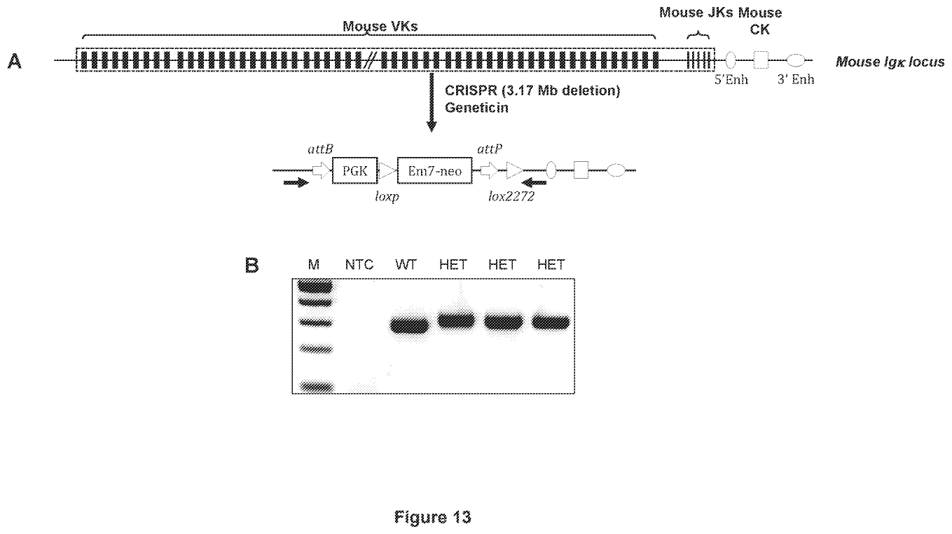

To remove the kappa light chains, a 3.17 Mb genomic DNA fragment that contains the entire mouse VK and JK gene segments was deleted by CRISPR/Cas9 mediated HDR and replaced with a docking cassette (FIG. 13A). Similar to the Singularity HyperDock at the Igh allele, the engineered Igκ HyperDock/KO allele contains an attB site, a PGK promoter, a loxP site, an Em7-neo cassette, an attP site, and a lox2272 site upstream of the 5′ Enhancer element located at the 5′ end of the mouse CK gene, thus allowing sequential RMCE at the Igλ allele. The engineered. The Igλ HyperDock/KO mice were generated and confirmed by PCR and sequencing (FIG. 13B).

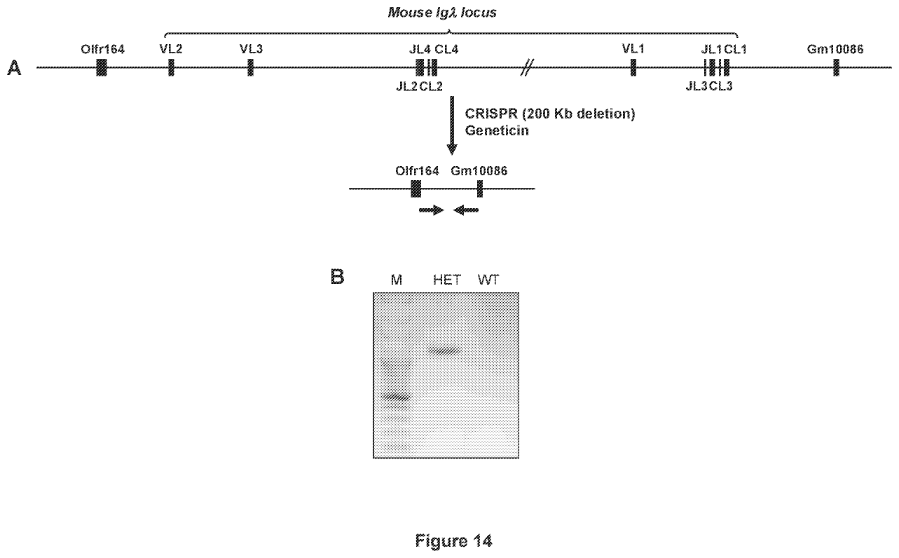

To remove the lambda light chains, a ˜200 kb genomic DNA fragment between Olfr164 and Gm10086, which contains the entire k locus including VL1, VL2, VL3, and all of JL and CL gene segments, was deleted by CRISPR/Cas9 mediated NHEJ (FIG. 14A). The Igl KO ES cells were generated and confirmed by PCR and sequencing (FIG. 14B).

### Example 2—Characterization of Singularity Mice

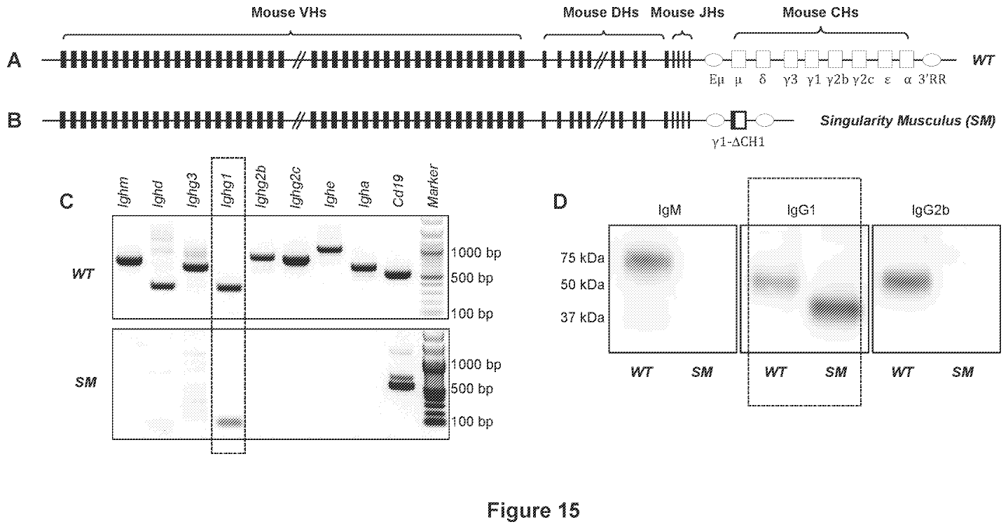

Singularity mice constitutively express only heavy chain antibodies of CH1-truncated IgG1 To confirm that, unlike WT mice that can express the full panel of Ig isotypes (FIG. 15A), the Singularity Musculus mice only express IgG1-ΔCH1 (FIG. 15B), transcription of different Ig classes and subtypes was analyzed. Total RNA was isolated from spleens of WT and Singularity Musculus mice using the Trizol reagent, and RNA concentration and quality was determined with Bioanalyzer. Reverse transcription was performed using Superscript IV Reverse Transcriptase (ThermoFisher) and oligo(dT) 20 primers according to the manufacturer's instructions. Transcription of all Ig classes and subtypes was analyzed by RT-PCR according to standard procedures, with mouse B-cell marker Cd19 as an internal control. While Ighm, Ighd, Ighg3, Ighg1, Ighg2b, Ighg2c, Ighe, and Igha were all detected in the WT mice, the Singularity musculus mice only expressed Ighg1 transcripts of reduced size (FIG. 15C), lacking the CH1 sequence as verified with sequencing.

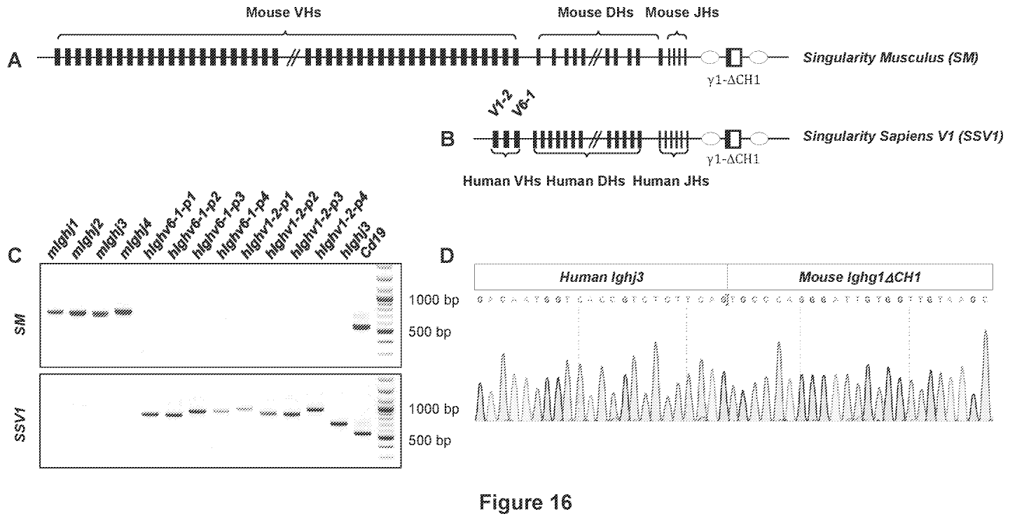

To examine the transcription of Ighg1 in Singularity Sapiens mice, RT-PCR was performed on cDNA that was reverse-transcribed from total spleen RNA of Singularity Sapiens (SSV1) mice. SSV1 derived from the Singularity Musculus platform (FIG. 16A) was designed to contain the full panel of human DH and DJ and two functional human VH (IGHV6-1, IGHV1-2) (FIG. 16B). PCR primers were designed having a set of forward primers specific to human IGHV6-1, IGHV1-2, and IGHJ3 and a reverse primer specific to mouse Ighg1 CH2. Chimeric transcripts with human VDJ-mouse Ighg1-ΔCH1 were detected in the Singularity Sapiens (SSV1), but not in the Singularity Musculus mice (FIG. 16C), which were further verified by sequencing to be correctly spliced (FIG. 16D).

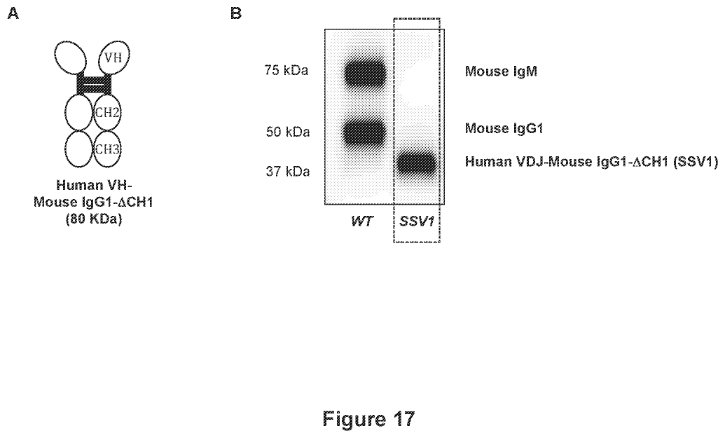

To examine the protein expression of different Ig classes and subtypes, immunoglobulins in plasma samples of immunized wild type and Singularity Musculus mice were purified with protein A/G magnetic beads, separated by reducing SDS-PAGE, and electrophoretically transferred onto Immobilon®-P membranes (Millipore Sigma) according to standard procedures. Immunodetection was conducted with HRP-conjugated secondary antibodies and enhanced chemiluminescence and autoradiography were performed using ECL Western blotting reagents. A truncated IgG1 of about 40 kDa was detected in Singularity Musculus mice (as compared to the full length IgG1 of about 50 kDa in the wild type mice), whereas IgM and IgG2b were not detectable (FIG. 15D). Similarly, a truncated IgG1 of about 40 kDa, corresponding to the human VDJ-mouse IgG1-ΔCH1, was detected in immunized Singularity Sapiens mice (SSV1), which did not express IgM or full length IgG1 as seen in the wild type mice (FIG. 17).

### Upregulated IgG Expression in B Cells of Singularity Mice

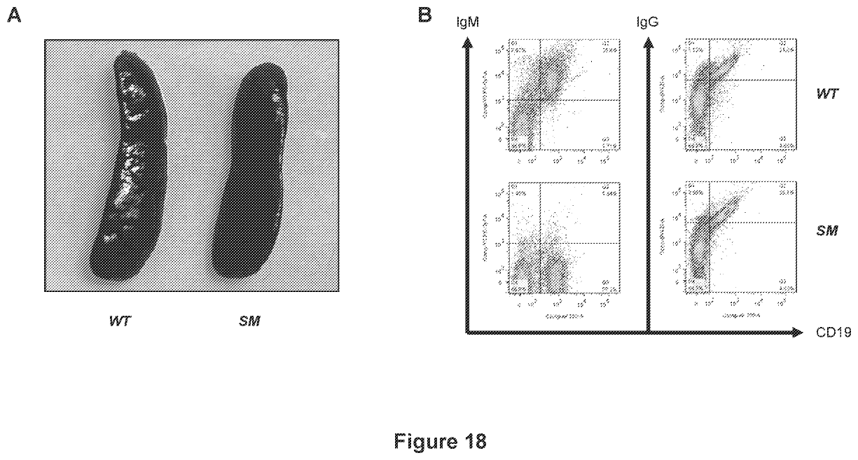

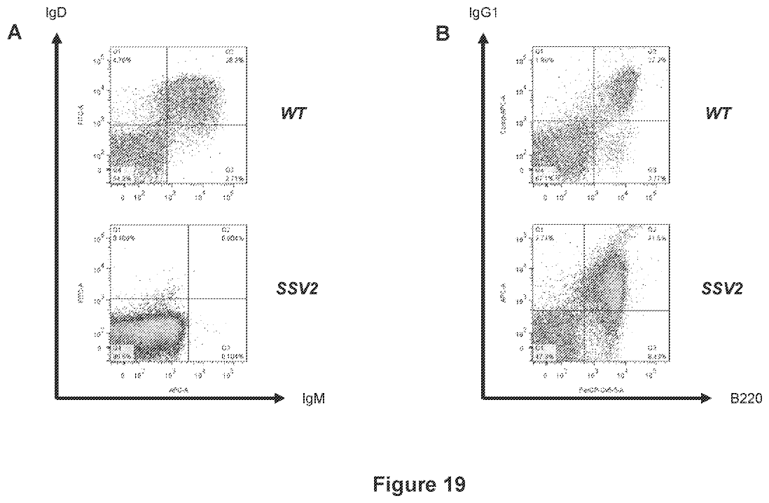

The spleens of Singularity Musculus mice were of similar shape and size as those of the wild type mice (FIG. 18A). To examine the expression of IgM and IgG on B cell membranes, single cell suspensions were prepared from spleens, treated with ACK lysis buffer to remove red blood cells, blocked with Fc blocker, and stained in FACS buffer (PBS with 1% FBS) with rat-anti-mouse IgM (PE-Cγ7), rat-anti-mouse IgG (BV421), and rat-anti-mouse CD19 (AF700). Following staining, cells were analyzed by flow cytometry (BD LSR II). While no IgM⁺ B cells were detected in the Singularity Musculus mice, IgG⁺ B cells were detected at significantly higher proportion as compared to those in the wild type mice, consistent with the increased expression levels due to the constitutive high-level germline transcription of IgG1 (FIG. 18B). Similarly, FACS analysis of splenocytes from Singularity Sapiens (SSV2) and wild type mice was performed using the above mentioned procedure with rat-anti-mouse IgM (APC), rat-anti-mouse IgG1 (APC), rat-anti-mouse IgD (FITC), and rat-anti-mouse B220 (PerCP-Cγ5.5). The analysis did not detect IgM⁺ or IgD⁺ B cells in SSV2 mice but they were found to be abundant in the wild type mice (FIG. 19A). IgG1 cells were detected at significantly higher proportion in SSV2 mice (FIG. 19B).

### Robust Humoral Immune Response in Singularity Mice

Protein antigens (Table 3) were prepared in phosphate-buffered saline (PBS) and freshly mixed 1:1 (v/v) with either Complete Freund's Adjuvant (Sigma Cat #5881, for priming injection) or Incomplete Freund's Adjuvant (Sigma Cat #5506, for boosting injections) by repeated passage through two connected syringes until a smooth emulsion was formed. Injections were performed using a 1-mL syringe and a 27-gauge needle into 4-12-week-old male or female mice. Priming and boosting injections were done at 2-week intervals at 10-25 μg antigen protein/mouse, subcutaneously into the left and right groin each (50 μL) and/or 100 μL intraperitoneally. Tail vein bleed was collected prior to each injection. A final boost was done in Week 4 or Week 6 intraperitoneally with antigen proteins without adjuvants. Animals were sacrificed 3-4 days later for terminal bleed and tissue harvest. Blood samples were processed into plasma according to standard procedures.

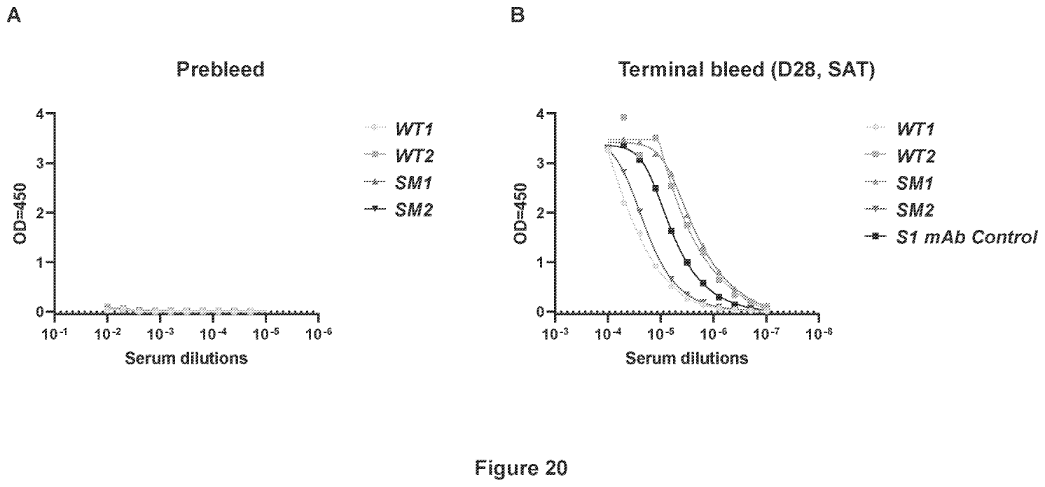

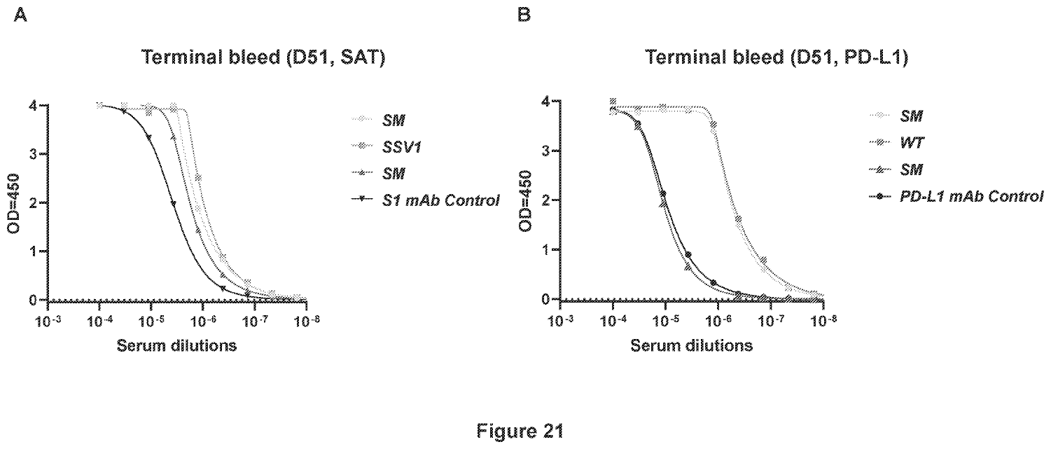

To assay for antibody titers in plasma, ELISA plates were coated with 1 μg/mL antigen protein diluted in PBS overnight at 4° C. Following repeated washing with PBST (PBS+0.05% Tween-20) and blocking with SuperBlock (Thermo Fisher), plasma samples were serially diluted in dilution buffer and applied to the plates. After unbound protein was removed through multiple washes, bound proteins were detected using a corresponding HRP-conjugated secondary antibody, developed with 3,3′,5,5′ tetramethylbenzidine (TMB) substrate (BM blue, Sigma) and stopped with 50 μL of 1 M H₂SO₄. Absorbance was read at 450 nm. Robust humoral immune responses comparable to those in wild type mice were observed in Singularity Musculus mice after 4 weeks (D28) with SAT immunization (FIGS. 20A and 20B), with significantly higher titers obtained after 6 weeks (D51) for both Singularity Musculus and Singularity Sapiens mice (SSV1) (FIG. 21A). Similar results were obtained with other immunogens such as PD-L1 (FIG. 21B).

### NGS Analysis of VH Repertoire in Singularity Mice

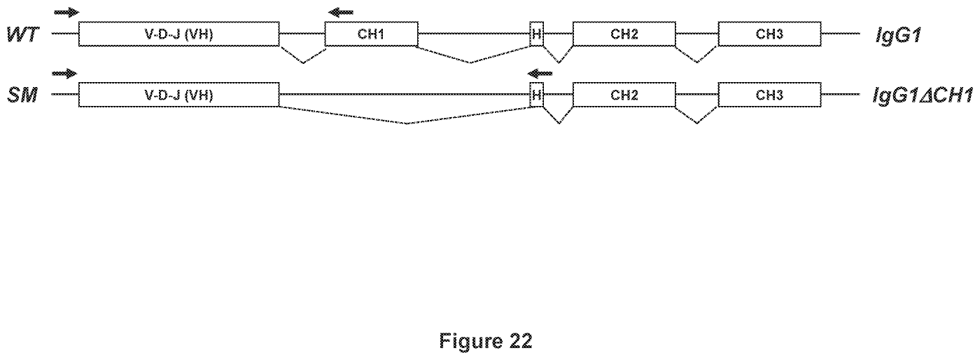

Total RNA was isolated by the Trizol reagent from spleens of wild type or Singularity Musculus mice immunized with different antigens, and RNA quality and concentration was determined with Bioanalyzer. The recombined variable region sequences (VH) of the wild type or Singularity mice were amplified by 5′ Rapid Amplification of cDNA Ends (5′RACE) for next generation sequencing (NGS). Briefly, reverse transcription was performed using Superscript IV Reverse Transcriptase, oligo(dT) 20 primers and a template switch primer that contains the 5′RACE adapter and unique molecular identifier (UMI) sequences (5′-CTACA-CTCTTTCCCTACACGACGCTCTTCCGATCTNNNNNNNNNNNNrGrGrGrGrG-3′; SEQ ID NO:37). The products of template switch reverse transcription were then amplified in the first round of PCR reaction using the 5′RACE adapter forward primer (5′-CTACACTC-TTTCCCTACACGACGCTCTTCCGATCT-3′; SEQ ID NO:38) and a reverse primer specific to the IgG1 CH2 domain (5′-GGTGGTTGTGCAGGCCCTCATG-3′; SEQ ID NO:39). Purified products were further amplified in the second round of PCR using the 5′RACE adapter forward primer and a reverse primer specific either to the IgG1 CH1 domain (5′-CCATGGAGTTAGTTTGGGCAGCA-3′ for wild type IgG1 transcripts; SEQ ID NO:40) or the IgG1 Hinge domain (5′-CAAGGCTTACAACCACAATCCCT-3′ for Singularity IgG1 transcripts; SEQ ID NO:41) to ensure that both mouse lines generated about 600 bp amplicons (FIG. 22). The resulting nested PCR products were further amplified in the third round of PCR reactions to incorporate the Illumina P5 and P7 adaptor sequences for NGS and barcodes to enable sample multiplexing. The final 5′RACE libraries were purified and sequenced using 2×300 bp paired-end run on an Illumina MiSeq.

Paired-end sequence reads in fastq format were processed and aligned using KAligner, a specialized version of K-mer chaining algorithm (Liao et al., Nucleic Acids Res., 41(10):e108 (2013)) to reference germline genes of VH, DH, and JH based on annotations from the international ImMunoGeneTics information system (IMGT, World Wide Web at imgt.org), and the CDRs regions were identified. The full-length in-frame sequences were further assembled into clonotypes when the CDR3 were identical and no more than 2 mismatched nucleotide residues were present among the sequences. The sequences with low quality were excluded from assembling. The clonotypes from each animal were ranked according to their abundance, and the clonotypes with less than 5 counts were not included for further analysis.

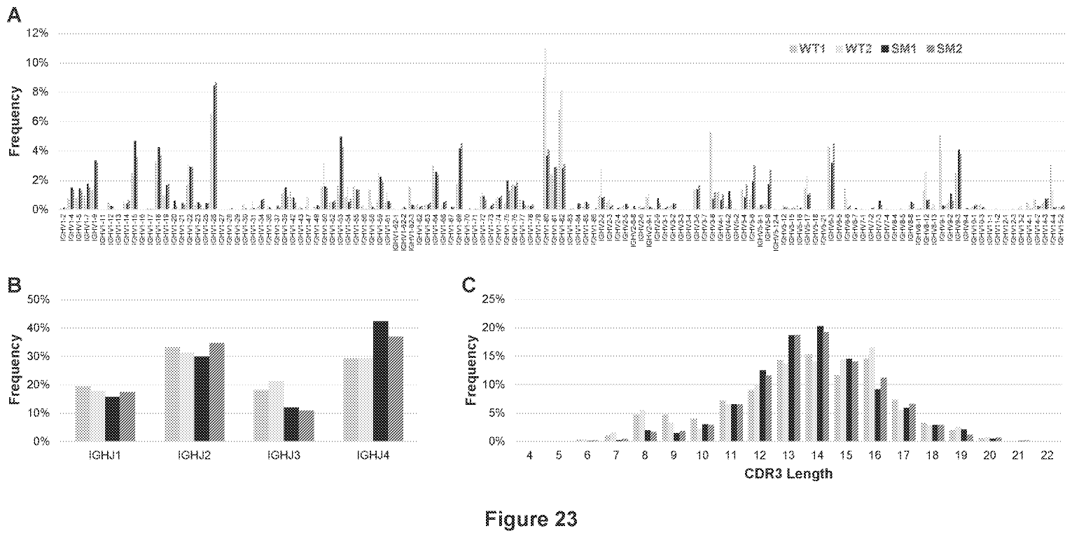

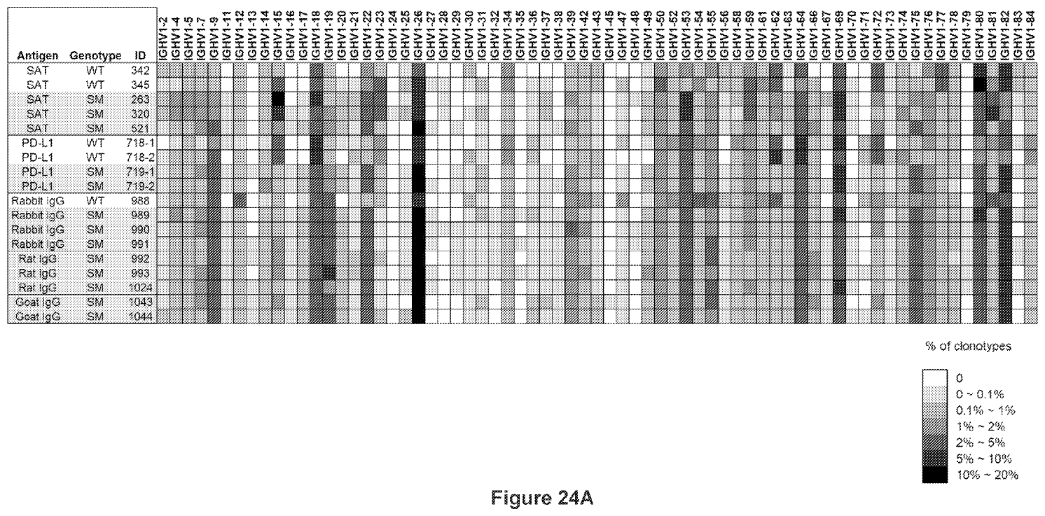

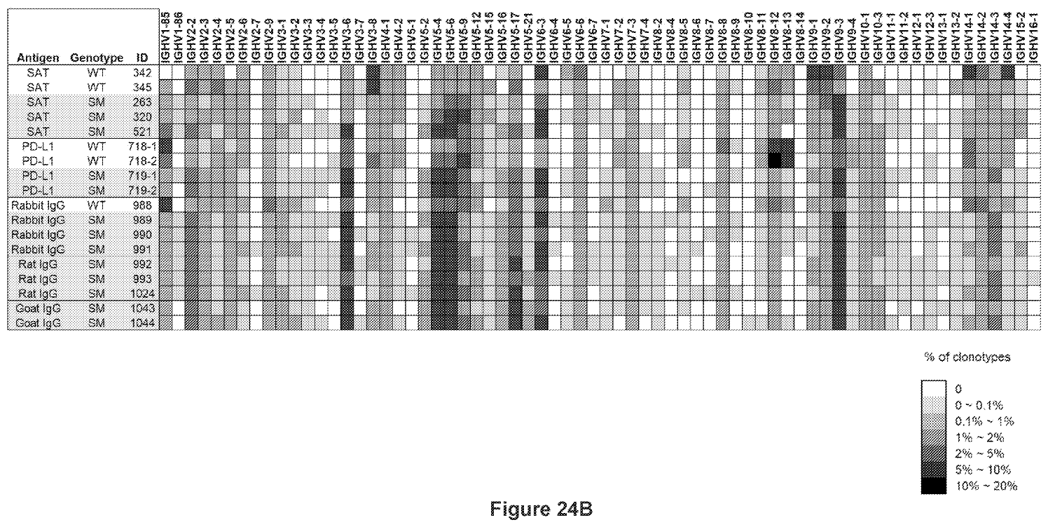

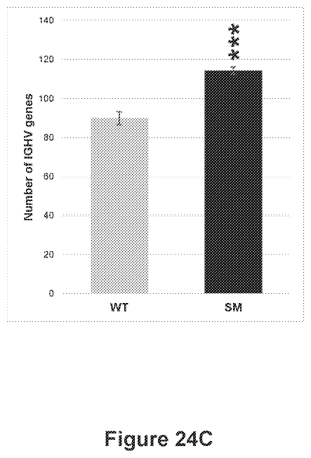

A total of 18 samples from wild type and Singularity Musculus mice immunized with SARS-Cov2 Spike Active Trimer SAT (R&D Systems; Catalog No. 10549-CV), PD-L1 (R&D Systems; Catalog No. 156-B7), Rabbit IgG (ThermoFisher; Catalog No. 02-6102), Rat IgG (ThermoFisher; Catalog No. 31933), or Goat IgG (ThermoFisher; Catalog No. 31245) were processed for NGS to determine their corresponding VH repertoire. More than one million reads per sample was recovered in all samples, roughly half of which successfully aligned to the Igh locus (Table 3). Notably, the number of clonotypes against all tested antigens in the Singularity Musculus mice were significantly higher than those seen in the WT mice, ranging from several fold to over 20-fold. Moreover, whereas 79-99 IGHV gene segments were utilized in WT mice against these antigens, a significantly higher number of IGHVs (103-122) were utilized in the Singularity Musculus mice, almost close to the theoretical limit (125 functional IGHVs based on mouse genome GRCm38/mm10 annotation) (FIGS. 23A and 24 and Table 3). The ability of the Singularity Musculus mice to access a greater number of IGHV segments as compared to WT was highly significant across several tested antigens (FIG. 24), which may have resulted from the high level of IgG1 GLTs in the Singularity Musculus mice as compared to the inducible, cytokine-dependent expression of IgG1 GLTs in the WT mice, as well as the removal of all other Ig classes and subtypes, thereby enabling sole expression of IgG1 regardless of immunogens.

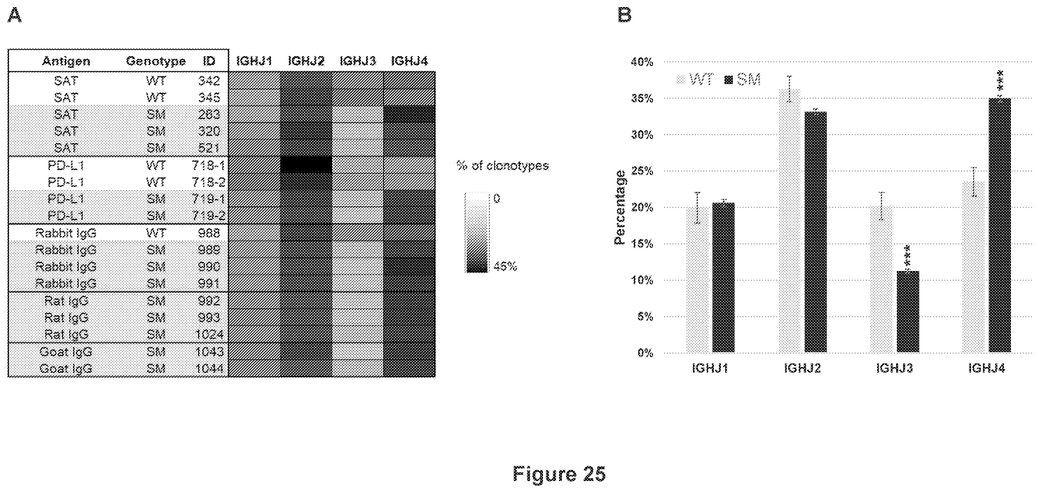

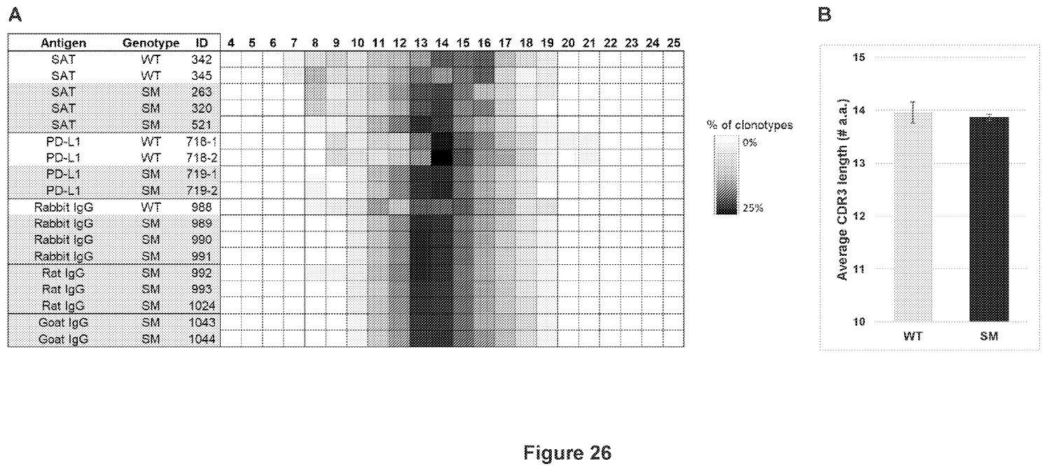

Analysis of VH sequences showed that compared to the WT mice, the Singularity mice exhibited similar diverse usage of the IGHV gene segments (FIGS. 23A and 24A-24B). The IGHV gene segments that yielded more abundant or less abundant clonotypes in the wild types also did so in the Singularity Musculus mice (FIGS. 24A-24B). While all 4 IghJ segments were used, IGHJ3 was discriminated whereas IGHJ4 was favored in Singularity Musculus mice, likely resulting from structural preference for HCAb formation (FIGS. 23B and 25). No significant difference was observed in CDR3 size distribution among clonotypes, with average size being about 14 for both wild type and Singularity Musculus mice (including the invariable C and W residues at the CDR3 boundaries) (FIGS. 23C and 26).

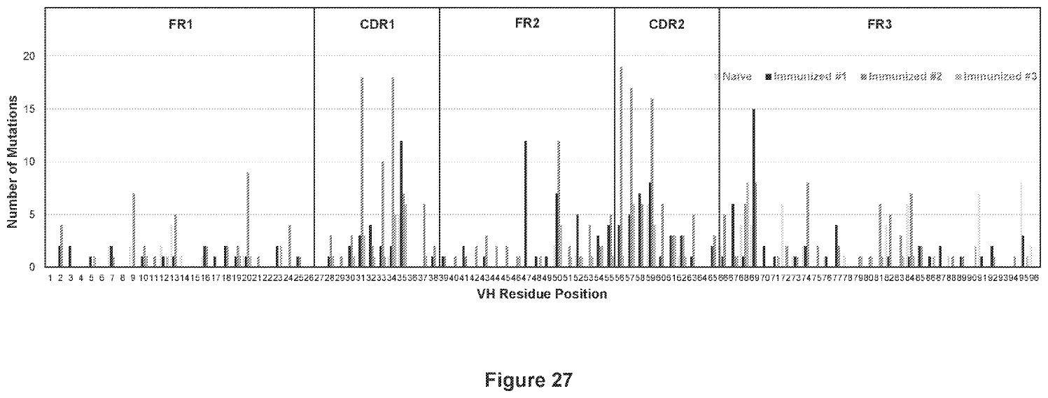

To identify somatic hypermutations, the top 100 ranked clonotype sequences from each Singularity Musculus mouse that were either naive or immunized with SAT were aligned to the corresponding germline IGHV sequences using IgBlast (World Wide Web at ncbi.nlm.nih.gov/igblast/). The mutation rate at each residue position according to the IMGT numbering scheme was calculated and plotted (FIG. 27). While a low level of mutation rate was observed in the naive mice, immunized mice exhibited a significantly higher level of somatic hypermutations, which were highly enriched in the CDR regions (FIG. 27). This was further confirmed in later analysis of the complete VH sequences of validated nanobody binders (FIG. 37).

### Example 3—Nanobody Discovery with Singularity Mice

### Sequence-Driven, High-Throughput Screen for Nanobody Binders

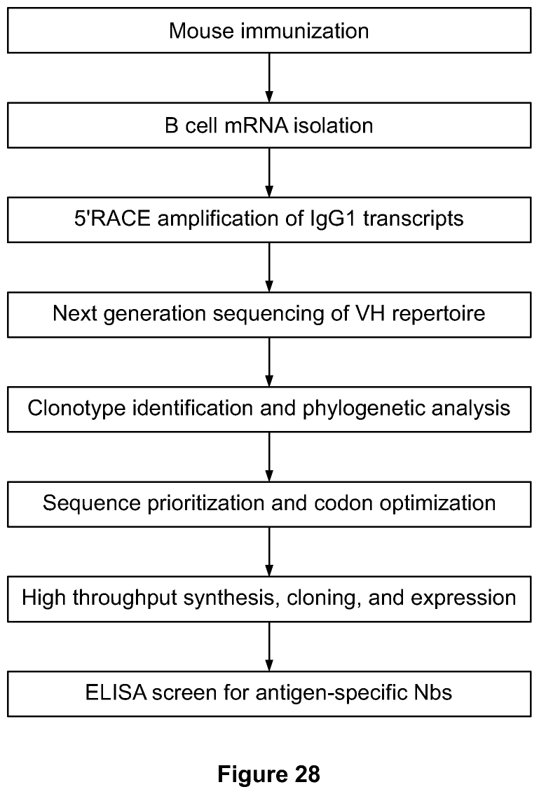

Next generation sequencing (NGS) and bioinformatic analysis was performed to profile and select VH sequences (clonotypes) from immunized Singularity mice, followed by gene synthesis, cloning, expression, and ELISA screening to identify nanobody binders (FIG. 28). Alternatively, nanobody binders can be identified with other methods including, without limitation, hybridoma, single B cell cloning, single B cell sequencing, and various display approaches such as bacterial display, yeast display, mammalian cell display, and phage display.

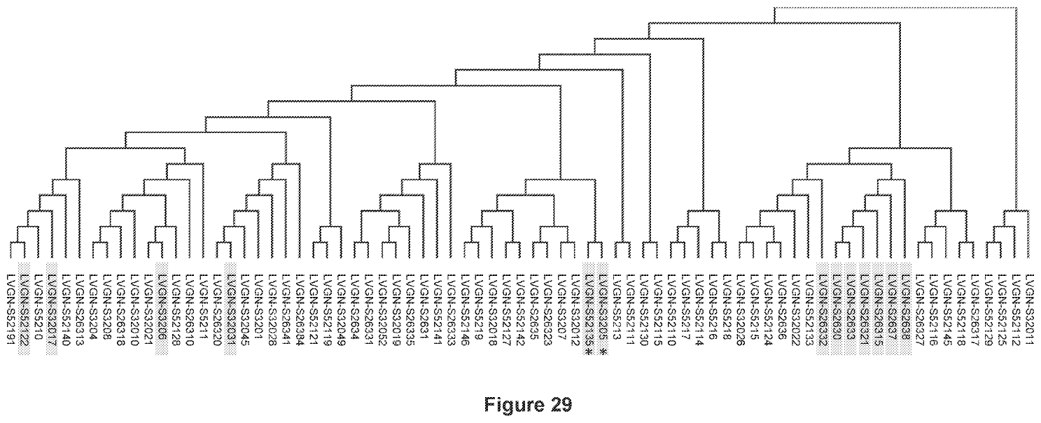

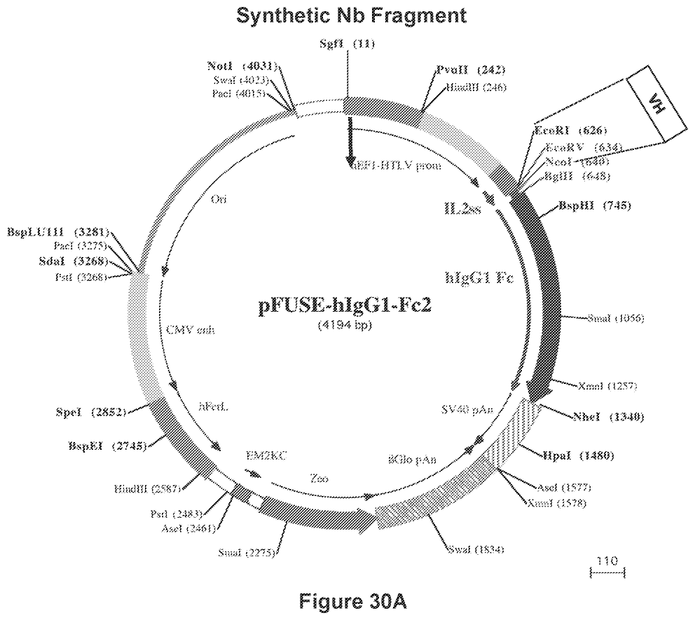

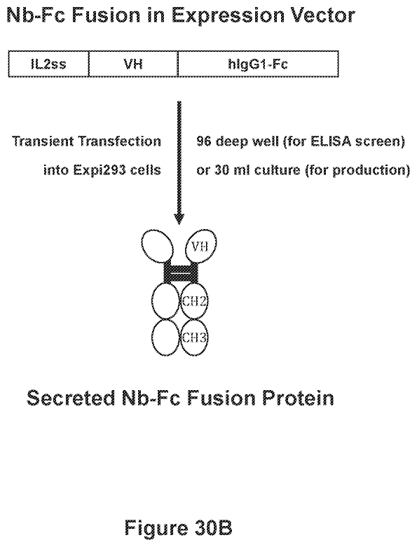

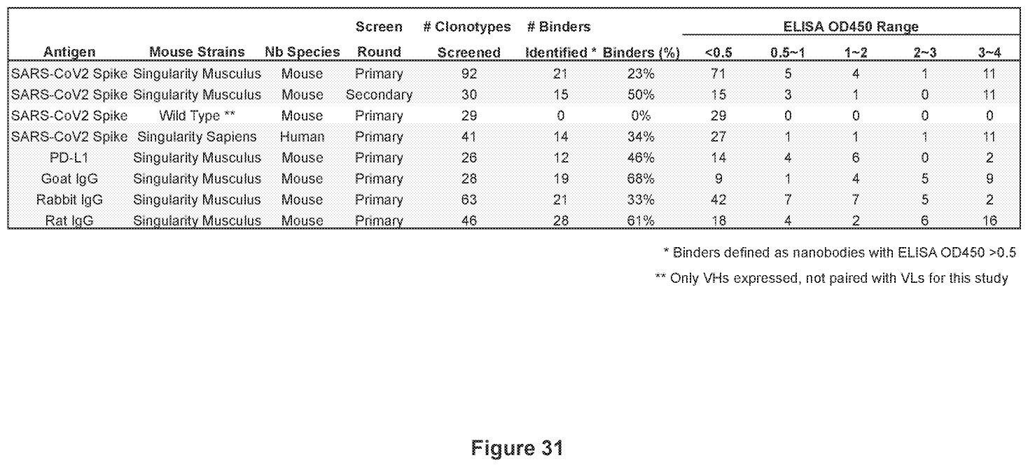

To select for candidate clonotypes for nanobody expression and binder screens, clonotypes from each animal were ranked according to their abundance and somatic hypermutation rate. Phylogenetic analysis of clonotype sequences was carried out with Clustal Omega (World Wide Web at ebi.ac.uk/Tools/msa/clustalo) to select representative sequences from different branches (FIG. 29). Candidate clonotype sequences (VHs) from the Singularity mice immunized with SAT were first human-codon optimized, flanked with cloning adapters, and synthesized as eBlock gene fragments (IDT). The synthesized eBlocks were then cloned into pFuse-hIgG1-Fc2 vector (Invivogen) by NEBuilder HiFi DNA assembly to generate in-frame fusions of an IL-2 signal peptide, VH, and the Fc domain (Hinge-CH2-CH3) of human IgG1 (FIGS. 30A-30B). Sequence-verified expression constructs were then transfected into Expi293F cells (ThermoFisher) in 96-well format to produced secreted nanobody-Fc fusions according to the manufacturer's instructions, and the culture supernatants were collected 6 days post-transfection and were used for ELISA screen to identify antigen-specific binders.

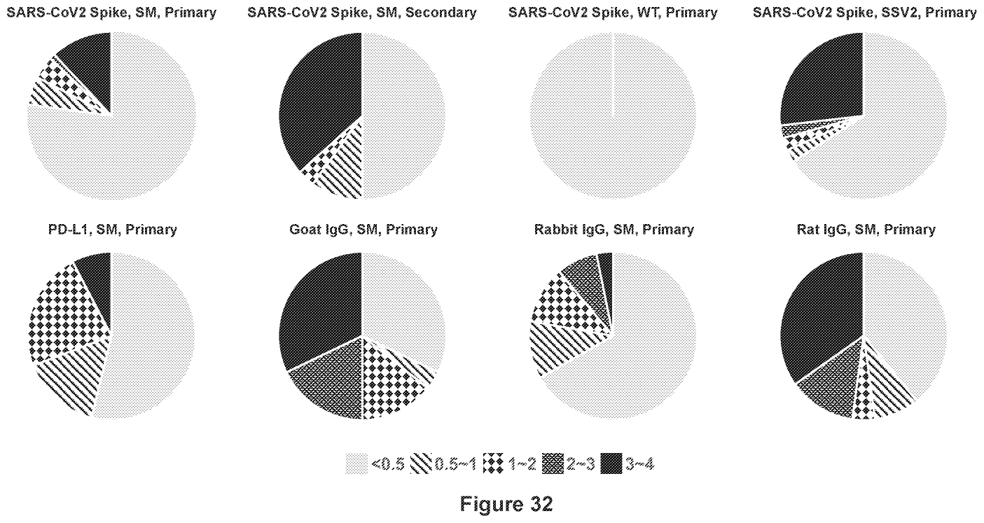

A screen of 92 clonotypes selected from Singularity Musculus mice immunized with SAT identified 21 (23%) binders (ELISA OD>0.5), among which 11 (52%) exhibited high level of binding (ELISA OD>3.0) (FIGS. 31-32). These VH sequences were then used to query the original clonotype sequence libraries by phylogenetic analysis to identify homologous VH sequences, which were then used for secondary screen for hit expansion. Fifteen (50%) of 30 clonotypes screened (mostly of low abundance thus not included in the primary screen) were binders, among which 11 (73%) exhibited high affinity (FIGS. 31-32). By contrast, a control screen using clonotypes from wild type mice selected after the same criteria (high abundance and hypermutation rate) failed to identify any binder (0/29), suggesting that functional nanobodies can only be derived from HCAbs produced in Singularity mice, but not possible from the conventional H2L2 antibodies produced in the wild type mice (FIGS. 31-32). SAT nanobody binder screen with Singularity Sapiens mice (SSV2) identified 14/41 (34%) Nb-Fc binders of human VH sequences, suggesting that functional HCAbs were produced in the humanized mice following the same mechanisms (FIGS. 31-32).

ELISA screens for nanobodies against PD-L1, Goat IgG, Rabbit IgG, and Rat IgG identified 33%-61% binders to the corresponding antigen, further demonstrating the high efficiency of the NGS-driven screening methods, which were uniquely suitable for nanobody discovery (FIGS. 31-32). Due to the single-chain nature of HCAbs, each clonotype identified represents a unique antibody, which was identified with bulk RNA-seq without using any single cell approach.

Biophysical and biochemical properties of purified Nb-Fcs

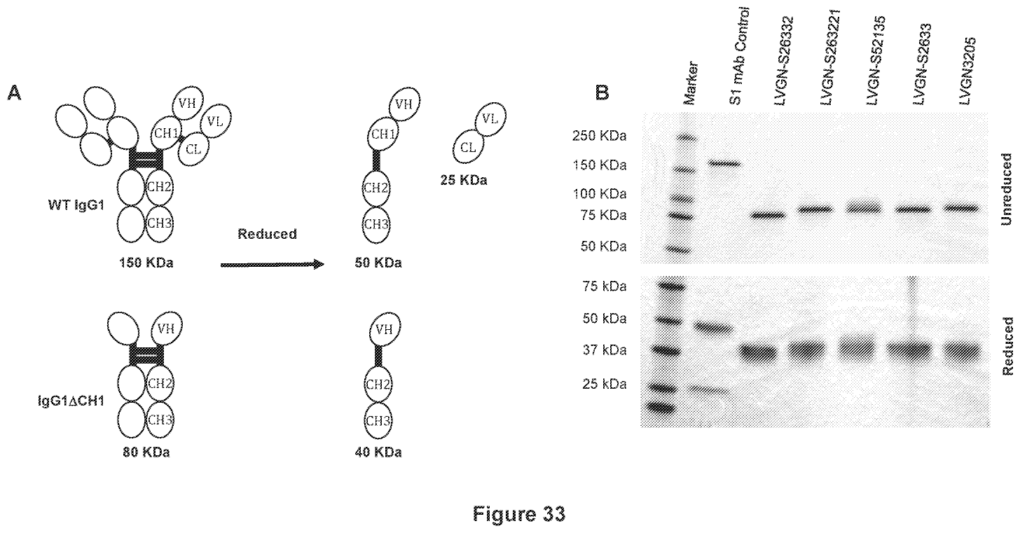

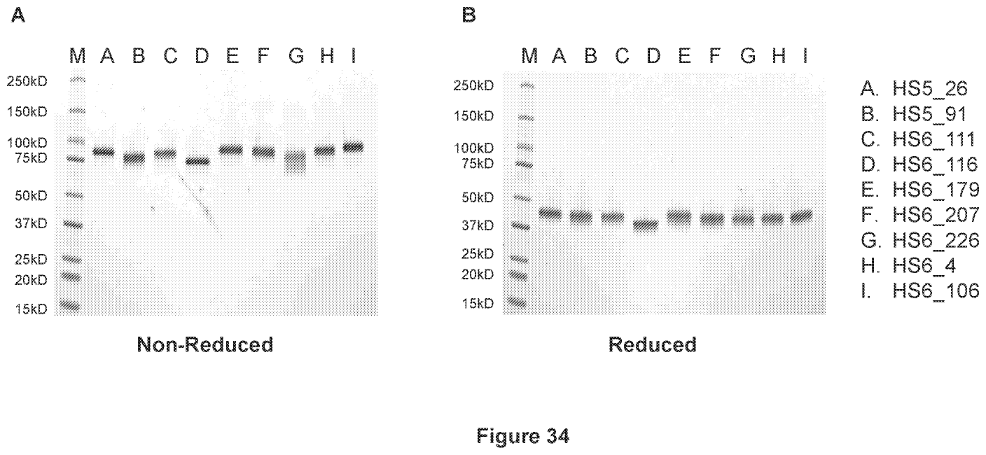

A selected set of ELISA positive SAT Nb-Fc constructs (from both mouse and human VH sequences; Table 5) were used to transfect a 30-mL Expi293F cell culture to produce nanobody-Fc fusions, which were then purified using protein A affinity chromatography following standard procedures. Briefly, six days post-transfection, cell culture supernatants were collected, filtered with 0.22 pm filters and loaded onto 0.5 mL protein A columns (MabSelect SuRe, Cytiva) pre-equilibrated with PBS. After washing with 2 mL of PBS, bound proteins were eluted from the columns with 4 mL citric buffer (25 mM citrated acid, 150 mM sodium chloride, pH 3.5) and neutralized with additional of 1 M Tris-HCl (pH 8.8). The final buffer was exchanged into PBS with Vivospin Turbo (30,000 MWCO PES). SDS-PAGE analysis showed that, in contrast to conventional antibodies (about 150 KDa) with two heavy chains (about 50 KDa) and two light chains (about 25 KDa), the purified Nb-Fc fusions migrated at about 80 kDa under unreduced condition and at about 40 kDa under reduced condition, consistent with the expected sizes of VH-based Nb-Fcs as homodimers (FIGS. 33-34).

TABLE 5
Biochemical and biophysical properties of mouse and human SAT Nbs.

| SARS-Cov2 | | | | | | | | | K_{D} (M) | | |
|---|---|---|---|---|---|---|---|---|---|---|---|
| Spike | | | | | Yield | Tm1 | ELISA | ELISA | SPR | SPR | BLI |
| Nb-Fc | Alt. ID | Species | PI | MW | (mg) | (° C.) | EC50 (M) | IC50 (M) | (Carterra) | (Biacore) | (Octet) |
| MS521_35 | LVGN-S52135 | Mouse | 7.19 | 82465.17 | 0.65 | 66 | 2.4E−11 | 4.9E−10 | 5.30E−09 | ND | 9.09E−09 |
| MS329_5 | LVGN-S3205 | Mouse | 7.19 | 82635.25 | 0.45 | 67 | 2.4E−11 | 4.0E−10 | 3.17E−09 | ND | 1.21E−09 |
| MS263_3 | LVGN-S2533 | Mouse | 8.49 | 83265.78 | 1.07 | 65 | 1.3E−03 | ND | ND | ND | ND |
| MS263_21 | LVGN-S26321 | Mouse | 8.33 | 83308.82 | 0.83 | 60 | 2.2E−10 | ND | ND | ND | ND |
| MS263_32 | LVGN-S26332 | Mouse | 7.75 | 81156.21 | 1.15 | 62 | 1.0E−10 | ND | ND | ND | ND |
| M2S521_193 | LVGN-S521193 | Mouse | 8.09 | 82405.21 | 0.31 | 65 | 8.5E−11 | 1.6E−10 | ND | ND | ND |
| M2S521_72 | LVGN-S52172 | Mouse | 7.74 | 82493.43 | 1.16 | 59 | 6.3E−11 | 4.1E−11 | ND | ND | ND |
| M2S320_377 | LVGN-S320377 | Mouse | 8.09 | 82531.31 | 1.56 | 66 | 9.7E−11 | 1.6E−9 | ND | ND | ND |
| M2S320_75 | LVGN-S32075 | Mouse | 7.19 | 82487.16 | 1.87 | 67 | 6.8E−11 | 1.2E−10 | ND | ND | ND |
| M2S521_378 | LVGN-S231378 | Mouse | 7.73 | 82525.39 | 0.92 | 68 | 8.1E−11 | 3.3E−9 | ND | ND | ND |
| M2S521_623 | LVGN-S521623 | Mouse | 7.19 | 82609.3 | 1.28 | 59 | 5.4E−11 | 1.9E−80 | ND | ND | ND |
| HS6_91 | NA | human | 8.76 | 83572.8 | 1.47 | 65 | 4.2E−11 | ND | ND | 4.80E−08 | 4.79E−08 |
| HS6_111 | NA | human | 8.77 | 81853.26 | 0.81 | 62 | 6.7E−11 | ND | ND | 1.20E−08 | 1.36E−08 |
| HS6_207 | NA | human | 8.53 | 82089.36 | 1.38 | 65 | 1.5E−10 | ND | ND | 1.10E−08 | 3.77E−08 |
| HS6_225 | NA | human | 8.65 | 82195.56 | 1.09 | 68 | 7.0E−11 | ND | ND | 4.20E−09 | 1.61E−08 |
| HS6_105 | NA | human | 8.66 | 81953.47 | 1.59 | 65 | 4.6E−11 | ND | ND | 4.80E−09 | 9.22E−09 |
| HS6_26 | NA | human | 8.65 | 53702.89 | 1.31 | 60 | 5.3E−10 | ND | ND | 1.10E−08 | 6.03E−08 |
| HS6_4 | NA | human | 8.66 | 81899.16 | 1.37 | 64 | 5.8E−10 | ND | ND | 3.12E−07 | 8.43E−08 |
| HAb5_S* | LVGN-S8107 | Human | 7.75 | 82769.74 | 0.97 | 65 | 1.8E−11 | 7.9E−11 | 3.73E−08 | 5.70E−09 | 1.98E−08 |

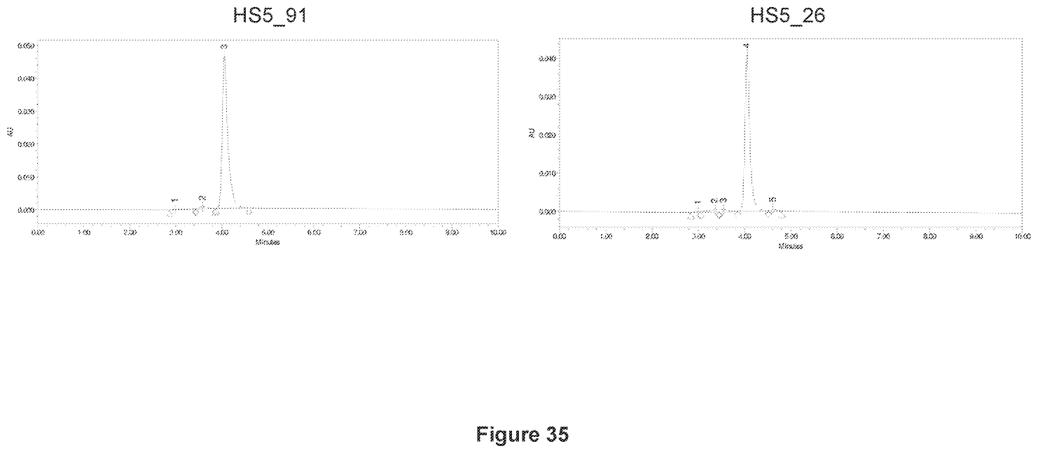

To examine the quality of purified Nb-Fcs, size exclusion chromatography (SEC) analysis was performed. Briefly, 2-10 μL purified Nb-Fc samples were injected into ACQUITY UPLC (Waters) Protein BEH SEC 200, 1.7 pm, 4.6×150 mm column with a flow of 0.3 mL/minute for 10 minutes. A mobile phase of 50 mM Sodium Phosphate, 500 mM NaCl, pH 6.2 was used. A high percentage of Nb-Fcs generated did not exhibit a propensity of aggregation (representative SEC plots on FIG. 35).

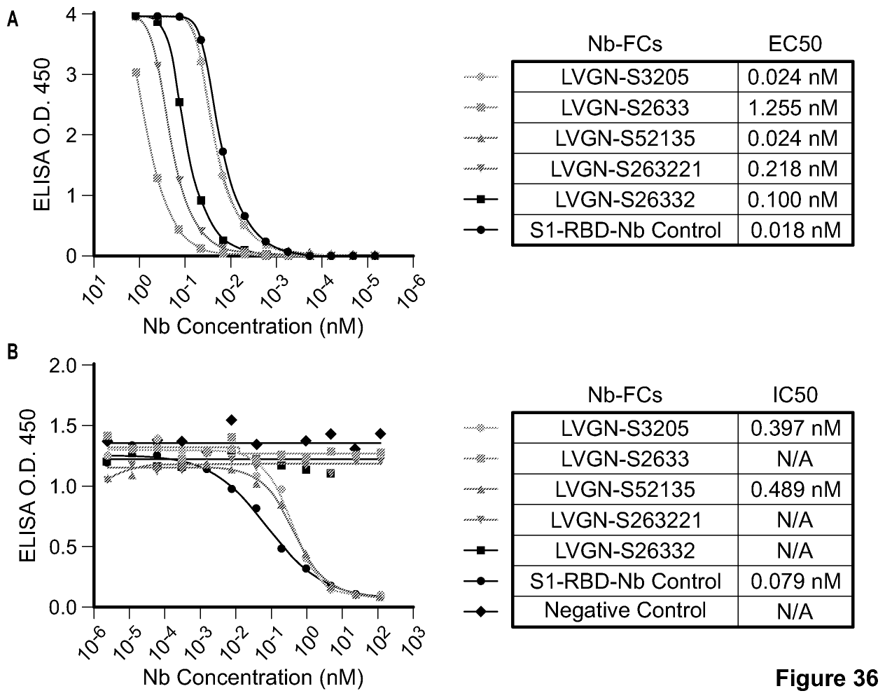

To assess binding affinity of purified SAT Nb-Fc fusions, ELISA assays were conducted with serially diluted protein samples against the SAT antigen. All Nb-Fc tested exhibited an ELISA EC₅₀ in the nanomolar and sub-nanomolar range, comparable to that of a human SAT Nb-Fc control VH-Ab-8 (HAb8-S) (Li et al., Cell, 183:429 (2020)) (FIG. 36A and Table 5). To access their neutralizing potency, a competitive ELISA assay was conducted with COVID-19 Spike-ACE2 Binding Assay Kit (Raybiotech) according to the manufacturer's instructions. A number of Nb-Fcs exhibited potent neutralizing potency against Spike-Ace2 binding, with IC₅₀ comparable to that of HAb8-S(FIG. 36B; Table 5).

Interestingly, many of those were identified from secondary screen using the two potent SAT neutralizing nanobodies (LVGN-53205 and LGVN-552135) identified from the primary screen, further demonstrating the power of the sequence-driven nanobody discovery pipeline (FIGS. 31 and 37).

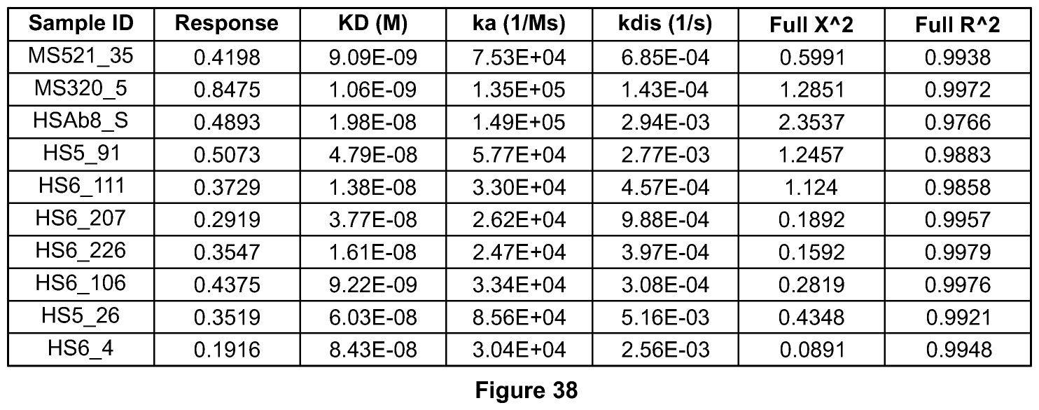

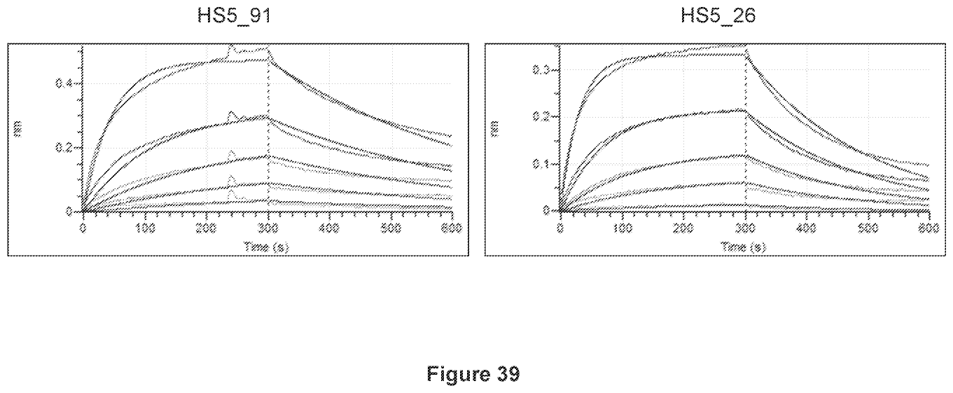

The kinetics of Nb-Fc to SAT binding was analyzed with Surface Plasmon Resonance (SPR) and/or Biolayer Interferometry (BLI) (Table 5). For BLI, binding experiments were performed on the Octet HTX at 25° C. The antibodies were loaded onto Anti-Human Fc Capture (AHC) sensors and then dipped with serial dilutions of antigen (starting at 333 nM, 1:3 dilution, 5 points). Reference sample well (buffer) was used for data analysis. Kinetic constants were calculated using a monovalent (1:1) binding model. Representative kinetics and sensorgrams are shown in FIGS. 38-39. Most Nb-Fcs exhibited a single or double digit nanomolar (10⁻⁸-10⁻⁹M) KD, comparable to that of the HAb8-S(Table 5).

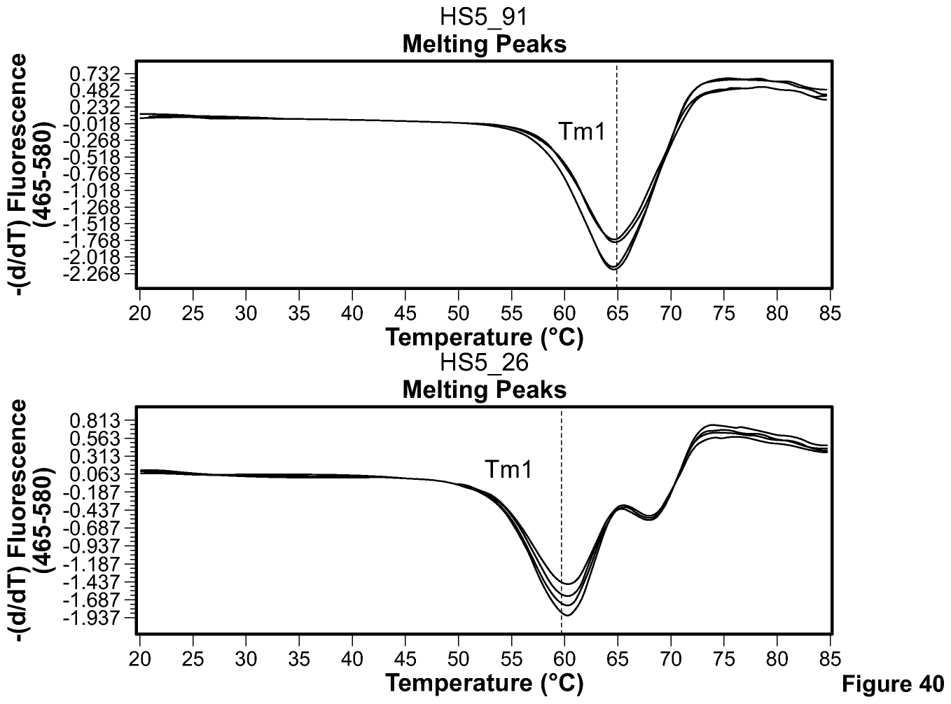

To assess thermostability, Differential Scanning Fluorimetry (DSF) was used to measure the melting temperature (Tm) of purified Nb-Fcs. Briefly, Nb-Fcs were mixed with Thermal Shift™ Dye (ThermoFisher) for a final concentration at 1 μg/mL, and a 10 μL/well mixture was transferred into a 384-well plate. The plate was sealed with MicroAmp® Optical Adhesive and loaded onto a Roche LightCycler® 480 Instrument. Fluorescence signals were collected as the temperature increased from 20° C. to 85° C. at 0.06° C./second. Most purified Nb-Fcs exhibited high thermostability, with an average Tm1=64.28±0.64° C. (PBS, pH7.4) (Table 5). Representative melting curves were shown in FIG. 40.

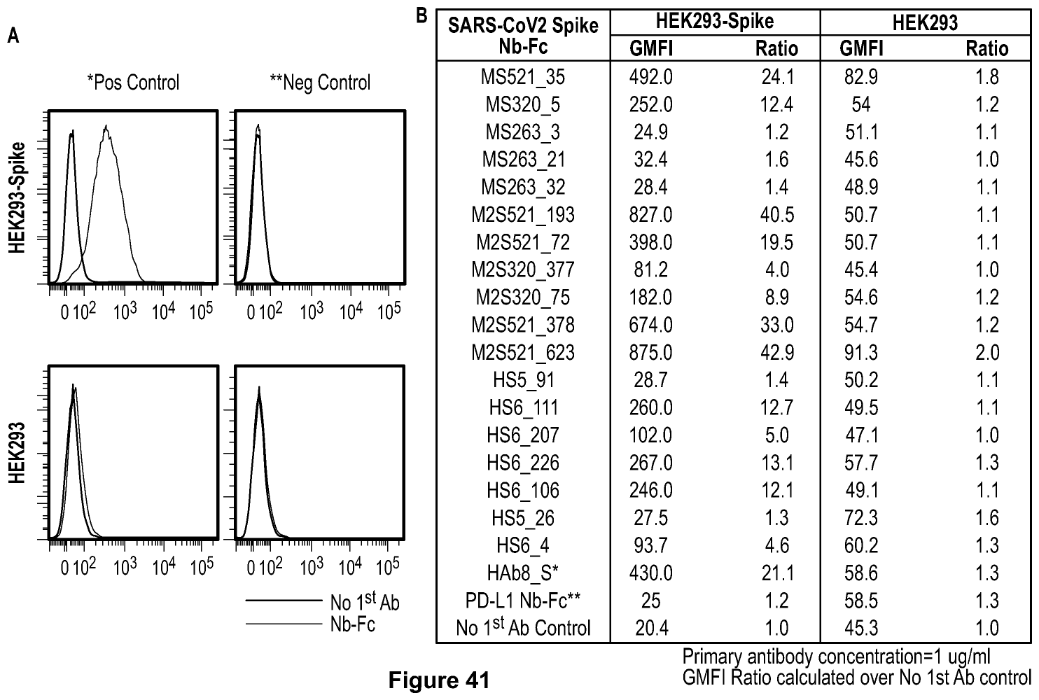

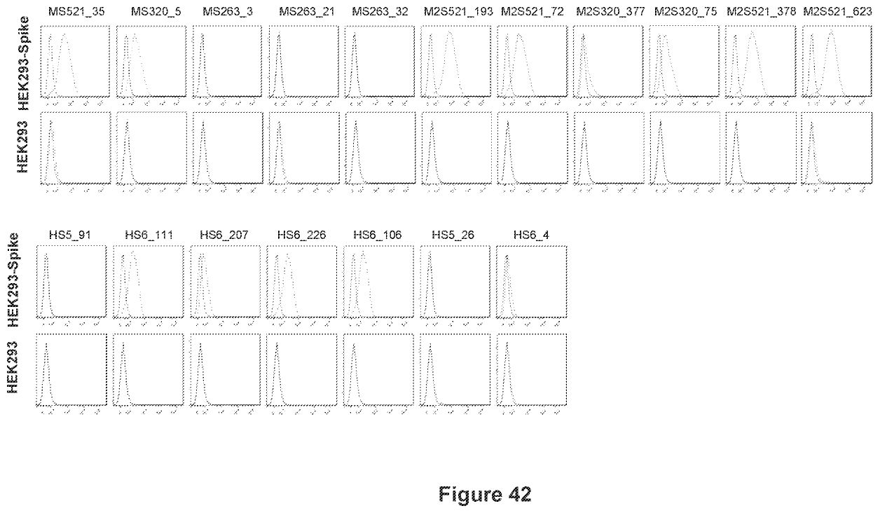

A FACS based cell binding assay was performed to examine the binding property of Nb-Fcs to Spike proteins on cell surface (FIGS. 41-42). HEK293 parental cells and HEK293-Spike cells expressing the SARS-CoV-2 Spike (S) protein with an inactivated furin site (293-SARS2-S-dfur, Invivogen #293-cov2-sdf) were incubated with individual Nb-Fcs at 1 μg/mL for 1 hour at 4° C., washed, and then incubated with goat anti-human IgG-Fc coupled to DyLight 594 (ThermoFisher) for 1 hour at 4° C. FACS analysis of these samples was performed on BD LSR II, and geometric mean fluorescence intensities (GMFI) were calculated with FlowJo V10. Twelve out 18 of Nb-Fcs, which bound to SAT in ELISA format, also exhibited cell surface SAT binding above background, with a GMFI ratio between 4.0-42.9 (FIGS. 41-42).

### Example 4—Additional Singularity-Based Non-Human Animals

### Generation of the Singularity Sapiens-L and -K Allelic Series

The variable light chain (V_{L}) gene segments contribute to the immune diversity of conventional tetrameric antibody. Human tetrameric antibodies contain either kappa (×) or lamda (λ) light chains. The human immunoglobulin light chain derives from two distinct loci: IGK and IGL on chromosome 2 and 22, respectively. Similar to the IGH locus, each locus encodes a large number of V_{L} gene segment. However, unlike the IGH locus, the light chain loci lack the diversity D gene segments; recombination at the light chain loci requires RAG1/RAG2 proteins but involves the joining of VL segment directly with a JL segment.

To access the unique properties of VL gene segments and enlarge the immune repertoire of the Singularity Sapiens platform, two separate approaches were used to exploit the diversity offered by the light chain variable gene segments. First, since the IGLV gene segments are flanked by 23RSS signals, which are similar to those of IGHV gene segments, they can properly pair up with the 12RSS signals immediately upstream of the IGHD gene segments satisfying the “12/23 rule” preferred by RAG1/RAG2 (FIG. 43A). For the lambda light chain genes, a Singularity Sapiens DJ-dock allele was first generated from the SSV1 allele by removing the human IGHV segments using CRISPR-Cas9 gene editing, leaving only human IGHD and IGHJ segments. Next, a panel of IGLV gene segments from a series of human BACs (CH17-262M19, CH17-329P5, CH17-238D3, CH17-261A15, CH17-264L24, CH17-117C7, RP11-1040J16, CH17-320F4) are modified (hLGLV-BACs) and are integrated sequentially via RMCE in a similar fashion as described previously (FIG. 43B).

Second, since IGKV gene segments are flanked with 12RSS signals that are not compatible with IGHD segments, a different approach is required to introduce the kappa light chain genes into the singularity allele. First, the Singularity HyperDock allele is used as a platform for the integration of modified human BACs (hIGKVJ-BACs) containing a series of VK and JK segments (CH17-272M2, CH17-405H5, CH17-140P2, CH17-13E7, CH17-84J8, CH17-53L15) (FIG. 44A). Alternatively, a Singularity Sapiens J dock allele was generated from the SSV1 allele by removing the human IGHV and IGHD segments using CRISPR-Cas9 gene editing, leaving only human IGHJ segments. Next, a panel of IGKV gene segments from engineered human BACs (hIGKV-BACs) derived from CH17-272M2, CH17-405H5, CH17-140P2, CH17-13E7, CH17-84J8, CH17-53L15 are integrated by sequential RMCE upstream of the IGHJ gene segments, allowing for recombination of individual IGKVs with each of the IGHJ gene segments that are flanked upstream by 23RSS signals (FIG. 44B).

Generation of the Singularity Longhorn and Minotaur alleles

Bovine antibodies are distinguished by the presence of ultralong complementarity-determining regions (CDRs) (Berens et al., Int. Immunol., 9(1):189-199 (1997)). These ultralong CDRs have antibody knob domains that can tightly engage their antigen as autonomous entities, which could generate ultra-small nanobodies of about 3-5 kDa in size (MacPherson et al., PLoS Biol., 18(9):e3000821 (2020)). CDR-H3s that range from 6-20 amino acids in humans and mice, can be as long as 50-70 residues in the cow. Ultralong CDR-H3s arise in part from the unusually long heavy diversity (D_{H}) gene segments encoded in the bovine germline genome (Shojaei et al., Mol. Immunol., 40(1):61-7 (2003); and Ma et al., J. Immunol., 196(10):4358-4366 (2016)). For instance, IGHD8-2 has 149 nucleotides and is one of the longest known D_{H}, contributing at least 50 amino acid residues to the bovine CDR-H3, and the combination of IGHV1-7, IGHD8-2, and IGHJ2-4 was predominantly found in isolated ultralong CDR3 bovine antibodies (MacPherson et al., PLoS Biol., 18(9):e3000821 (2020)).

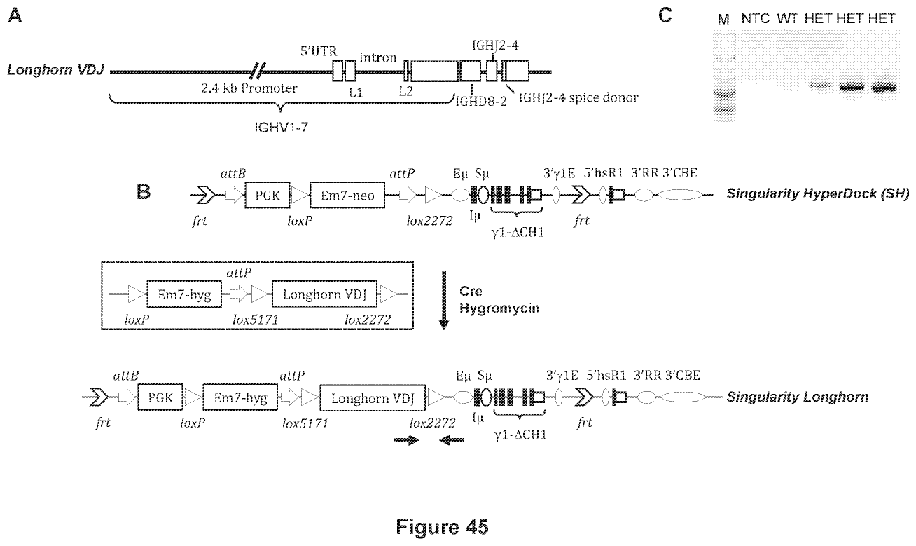

Building on the Singularity platform, a synthetic 3252 bp gene fragment was constructed that included the about 2.5 kb promoter plus 5′UTR region upstream of the bovine (Bos Taurus) IGHV1-7, the entire IGHV1-7 leader exon, intron, and coding sequences followed by IGHD8-2, IGHJ2-4, and 250 bp sequences immediately downstream of IGHJ2-4 region containing the splice donor sequences (FIG. 45A). The construct was integrated into the Singularity HyperDock allele via RMCE resulting in a mouse designated as Singularity Longhorn (FIG. 45B). PCR-genotyping and sequencing confirmed proper integration and transmittance of the Singularity Longhorn allele in F1 mice (FIG. 45C) from which the bovine VDJ-mouse IgG1ΔCH1 transcripts were detected.

Sequence of Longhorn VDJ (Bovine IGHV1-7
leaders and intron 1 is underlined, bovine
IGHV1-7 is bold, bovine IGHD8-2 is italic,
and bovine IGHJ2-4 is bold and underlined)
(SEQ ID NO: 57)
TGTCTTTTCTGTTTTTAATTTTATTCCAGTAAATTCATTTTCCTC
CTTTGGAGACTGGCTAAAGACTTATGGTTTTGTTTGTCTTTGAAA
AAACGACAATGACAACAACAACACCAGCTGTTGATTTCACTGATC
GTTGTTGCTGTTTTGTGTGTCTGTGTTCATCTCAATATTATTAAT
TTCTGCACTTTTTTGTCATTTCCTTCCTACTACTGCCTCTGTGCT
TTTGTGGTGTTCTGTTCTAATTTCTGTAGGTAGTAAGTTGGATTC
TTTCCTTCCATTTCTTGAAGAAGGCCTGTATCACTGTCAGCTTTC
GTTTCAGAACTGCTGGGCCGGCATAGAGTTTAGAGATAAGAGTGT
GTTAGTCAGTCAGTCGTGTCTGACACTTTGTTGCCCCAGAGACTG
TAGCTCGCCAGTCTTCTTTGTCCTTTCTAGGCAAGAATCCTGGAG
TGGCCTGCCATTTCCTACTCCAGGGAATCTCACACCTGGGGAGTC
AACCCAGGTCTCCTACATTGCAAGTGGATTCTTTACCACAGGAGG
CAAAATTCTCATTATCTACAGAACACTGTAGATAGAAAACCCTAA
AGACTCCACACAAAGCTATTAGACCACACTCATCCAGAGAGCACT
GAACTCAGTAAGTCAGAGTGGGTACCAGCATCTGATTGCTCATGG
CTTAGGAGTTGTTGGGGATTGTCTGGATCCCACGTCTGATGCCAT
AAACTAAACCCCATAGAAACCAACTGGTAGTGGATGTGGGGATGT
GTCTGCAGCCTTCCAGTCCCGTGAGATGAGAAGCATATTTCGGTT
TTACAGGTGGTCCAGTGGGATCACGAGATCAGAGAAACTGCTGAA
GAGTGGAAAGCCTGTTAGGCGTGCACCACAAGACTCCACTGGGTT
TGATGATGGATGAAGGTGTTGAGCCAAGGGATGAGGACACATGTA
GGAGCTCAGTGACCTCGATGCCAGAGGCCACAGTTCCAGGGTCAC
CCTCAGGAGTCTTGACCATGTTGCATGGCATCGGGCACAGGTGAC
CCCACAAGCTCTGAGGCAGAGTGAGGTGGGGCCTCTCCCCAGAGC
ACATTCAGGTGTCAGGAAACTGGATCTGGCACAAAAGACATGCTC
ATGCAGAACTGCTGTGACCAAAGCCAGACTGGGAAGGCCTTTTCA
TAGACAGTAGGTTCTCAGTCAGTGTACAGGTCAGATCCCTGTGCC
GAGGAAGAGCTGACCTGCACCAGACCCGAAGGGGTGACATCTCAG
GGCAGGAGAAGACCAGGGTCACATGGACAGGGCGTCAGTCAGAGT
GGGGACACACACTGGTCTTTTTTGCTCCAGGAGGGATAACACCAC
TGATGACATGATGACAGCACTCCCCACCCCGGGCAAACCCGCTGT
AAATCCACACATGCTGTGACTGTCGTCAGCCCCAGTCTACATCCT
CACACACAGAAATGCAGGTGTGAGGCCACAGCCGCAGGGCCACAG
GGAGATGGGGGAGCGGAGCCCTAAGGAGCTCCCAGGAGCTGTCTG
TCCCCACTCCTCAGCAGACTTGGGGTCCTCAGTGTCCACCCTCAG
GGAAGAGATGCTGAGGTTCACGAGTTCCTGCAGACACTCAGTTGT
GCACAGGAAAATGCAGCCCACTGGGCAAGTCAGCCGTGCTGGGAA
GACGAGCATCTCAGCACAAATGTCTACTGTGTTCACAGAATTAAT
ACTGCGCTTAATTACTATGAACAAACCTCCTCTAGATTGATTCTT
GAGGTGTTTAAGCTGTTCAAGATGAAAAAACAGTCAAGAAAGCAA
AGGAAAATATAAATGCTTTAGGAATTCTGATAGACATTAAATTGT
ACTCAACTCCACTTGAATTCAGTGTGATTATTACTTGTATATTTT
TCTCTGATGTTTGTATATGGAGTAAGTAAACATCAGCTAATGAGA
AAAGAAAAAAAAAATAAGTTTGTGTCTAACAAATGGCTGGTACCT
GAATGTGTGGTGGGCATTTGTTGAGTAACAGAAAACACATTCGTG
GGATTAAAGTCTCTTCCCCAAATCTGCCATTTTCCCTGGAAGCAT
TTTTTTCCTGACCAGCCCTTCTTCATCATTTCATTCCTCAAAGTC
TTCCTGGTGTGGGGAGGGGGATCCCAGGAAGGGCTGAGTTCTCTG
AGGCCACAGACAGCACCCCCTCACAGGGGGAGCCCAACACACAGG
ACCTCTATTCACTGCTTTCTGCTTTTTATACAGAGGTCCCTCCTG
TATGCAAATATCCACTCAAGTCACAGGGTAGAAACACAGCACCAG
CTCCCTTAAATTCAGGCTCCTCCTGAGGCTTGAAGAGACTTGTGG
GAGTGGTGACTCTCATCTGCTCCAAGATGAACCCACTGTGGACCC
TCCTCTTTGTGCTCTCAGCCCCCAGAGGTGAGTGTCTCTGGGTCA
GACATAGGCACGTGGGGAAGCTGCCTCTGAGCCCACGGGTCACCG
TGCTTCTCTCTCTCCACAGGGGTCCTGTCCCAGGTGCAGCTGCGG
GAGTCGGGCCCCAGCCTGGTGAAGCCGTCACAGACCCTCTCCCTC
ACCTGCACGGTCTCTGGATTCTCATTGAGCGACAAGGCTGTAGGC
TGGGTCCGCCAGGCTCCAGGGAAGGCGCTGGAGTGGCTCGGTGGT
ATAGACACTGGTGGAAGCACAGGCTATAACCCAGGCCTGAAATCC
CGGCTCAGCATCACCAAGGACAACTCCAAGAGCCAAGTCTCTCTG
TCAGTGAGCAGCGTGACAACTGAGGACTCGGCCACATACTACTGT
ACTACTGTGCACCAGAGTTGTCCTGATGGTTATAGTTATGGTTAT
GGTTGTGGTTATGGTTATGGTTGTAGTGGTTATGATTGTTATGGT
TATGGTGGTTATGGTGGTTATGGTGGTTATGGTTATAGTAGTTAT
AGTTATAGTTATACTTACGAATATTACGTCGATGCCTGGGGCCAA
GGACTCCTGGTCACCGTCTCCTCAGGTGAGTCCTCAACAGCCCTC
TCTCCTCACTCTCTCTCAGGGTTTTGGTGCACTTTGGGGAAAATC
GAGGGTGTCGGGTCTAAGGGGCCTGGGGCAGCCGGGGGTCTGAAA
CACTGAGGGCCCAGGGGCCCAGGCTTACAGCACCGAGGAGCAGAG
GCTCCAGGCACC

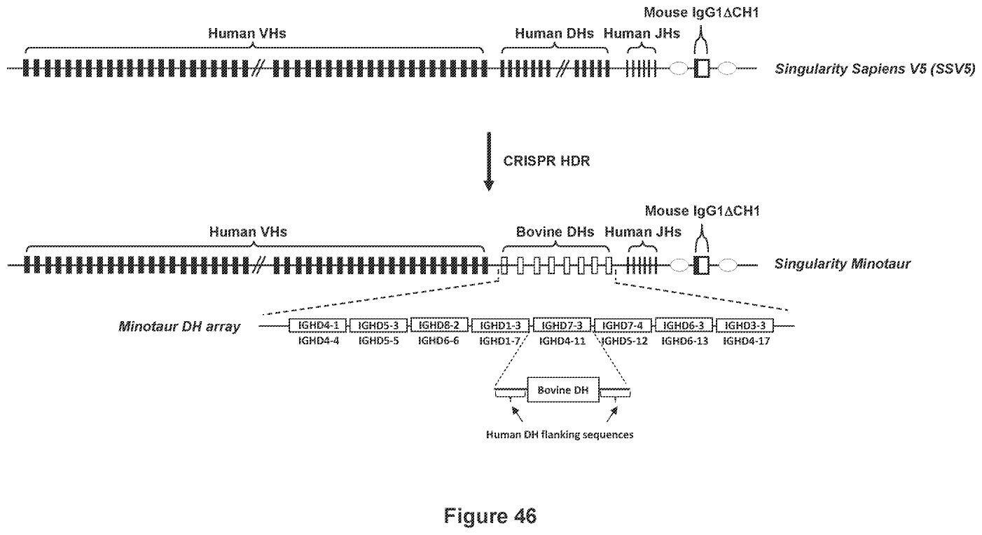

Based on the successful results demonstrating that the Singularity Longhorn mice were capable of expressing bovine-mouse chimeric heavy chain antibodies having the longest known DH gene segments, a synthetic array composed of eight of the longest bovine DH gene segments (IGHD4-1, IGHD5-3, IGHD8-2, IGHD1-3, IGHD7-3, IGHD7-4, IGHD6-3, and IGHD3-3) was constructed to replace the human IGHD component of the Singularity Sapiens mice. The sequences of the human framework were based on the genomic region spanning 550 bp upstream of IGHD4-4 to 550 bp downstream of IGHD4-17, with the intervening coding sequences of human DH genes replaced with bovine counterparts, while preserving all human genetic elements including the 12RSS signals (FIG. 46). The synthetic human-bovine D_{H} array contained the sequence below, that was used to replace the human IGHD gene fragments in the Singularity Sapiens alleles via CRISPR/Cas9 mediated HDR. Mice generated from these genetic engineering, referred to as Singularity Minotaur for human-cow hybrid, are designed to produce human nanobodies with ultralong CDR-H3s derived from Bos Taurus.

Sequence of Singularity Minotaur array
(Bovine IGHD is bold and underline)
1) Human: IGHD4-4; Bovine substitution: IGHD4-1
(SEQ ID NO: 42)
GACGCCTGGACCAGGGCCTGCGTGGGAAAGGCCTCTGGGCACACT
CAGGGGCTTTTTGTGAAGGGTCCTCCTACTGTGGTAGTTATAGTG
GTTATGGTTATGGTTATAGTTATGGTTATACCCACAGTGATGAAC
CCAGCAGCAAAAACTGACCGGACTCCCAAGGTTTATGCACACTTC
TCCGCTCAGAGCTCTCCAGG
2) Human: IGHD5-5; Bovine substitution: IGHD5-3
(SEQ ID NO: 43)
CTATTCCCTGGGAAGCTCCTCCTGACAGCCCCGCCTCCAGTTCCA
GGTGTGGTTATTGTCAGGGGGTGTCAGACTGTGATGATACGATAG
GTGTGGTTTTAGTTATTGTAGTGTTGCTACCACAGTGGTGCTGCC
CATAGCAGCAACCAGGCCAAGTAGACAGGCCCCTGCTGTGCAGCC
CCAGGCCTCCAGCTCACC
3) Human:IGHD6-6;Bovinesubstitution:IGHD8-2
(SEQ ID NO: 44)
GACCAAGTTGTGCTGAGCCCAGCAAGGGAAGGTCCCCAAACAAAC
CAGGAAGTTTCTGAAGGTGTCTGTGTCACAGTGGTAGTTGTCCTG
ATGGTTATAGTTATGGTTATGGTTGTGGTTATGGTTATGGTTGTA
GTGGTTATGATTGTTATGGTTATGGTGGTTATGGTGGTTATGGTG
GTTATGGTTATAGTAGTTATAGTTATAGTTATACTTACGAATATA
CCACAGTGACACTCGCCAGGCCAGAAACCCCATCCCAAGTCAGCG
GAATGCAGAGAGAGCAGGGAGGACATGTTTAGGA
4) Human: IGHD1-7; Bovine substitution: IGHD1-3
(SEQ ID NO: 45)
CACAAGCCCAGCCCCCACCCAGGAGGCCCCAGAGCACAGGGCGCC
CCGTCGGATTCTGAACAGCCCCGAGTCACAGTGAGACTATCGTGA
TGATGGTTACTGCTACACCCACAGTGACTCAGGCCCTGACATAAA
GTCTGACCCGCACACAGGTGTGGAGCTGGCCAATGCATCCCCAGG
GGCACTGGGCTCCCAAGCACTGTGAGAAAAGCTTCGTCCAAAACG
GTCTCCTGGCCACAGTCGGAGGCCCCGCCAGAGAGGGGAGCAGCC
ACCCC
5) Human: IGHD4-11; Bovine substitution: IGHD7-3
(SEQ ID NO: 46)
CAAATGCCTGGACCAGGGCCTGCGTGGGAAAGGTCTCTGGCCACA
CTCGGGCTTTTTGTGAAGGGCCCTCCTGCTGTGGTAGTTATGGTG
GTTATGGTTATGGTGGTTATGGTTGTTATGGTTATGGTTATGGTT
ATGGTTATACCATAGTGATGAACCCAGTGGCAAAAACTGGCTGGA
AACCCAGGGGCTGTGTGCACGCCTCAGCTTGGAGCTCTCCAGG
6) Human: IGHD5-12; Bovine substitution: IGHD7-4
(SEQ ID NO: 47)
CTGTTCCCTGGGAAGCTCCTCCTGACAGCCCCGCCTCCAGTTCCA
GGTGTGGTTATTGTCAGGCGATGTCAGACTGTGGTAGTTATGGTG
GTTATGGTTATGGTGGTTATGGTTGTTATGGTTATGGTTATGGTT
ATGGTTATGGTTATACCACAGTGGTGCCGCCCATAGCAGCAACCA
GGCCAAGTAGACAGGCCCCTGCTGCGCAGCCCCAGGCATCCACTT
CACC
7) Human: IGHD6-13; Bovine substitution: IGHD6-3
(SEQ ID NO: 48)
AACCAAGGGGTGTTGAGCCCAGCAAGGGAAGGCCCCCAAACAGAC
CAGGAGGTTTCTGAAGGTGTCTGTGTCACAGTGGTAGTTGTTATA
GTGGTTATGGTTATGGTTATGGTTGTGGTTATGGTTATGGTTATA
CCACAGTGACACTCACCCAGCCAGAAACCCCATTCCAAGTCAGCG
GAAGCAGAGAGAGCAGGGAGGACACGTTTAGGAT
8) Human: IGHD4-17; Bovine substitution: IGHD3-3
(SEQ ID NO: 49)
GTTACGATTTGACGCCTGGACCAGGGCCTGCGTGGGAAAGGCCGC
TGGGCACACTCAGGGGCTTTTTGTGAAGGCCCCTCCTACTGTGGT
ATTGTGGTAGCTATTGTGGTAGTTATTATGGTACCACAGTGATGA
AACTAGCAGCAAAAACTGGCCGGACACCCAGGGACCATGCACACT
TCTCAGCTTGGAGCTCTCCAGG

### Generation of the Singularity Sapacos Allelic Series

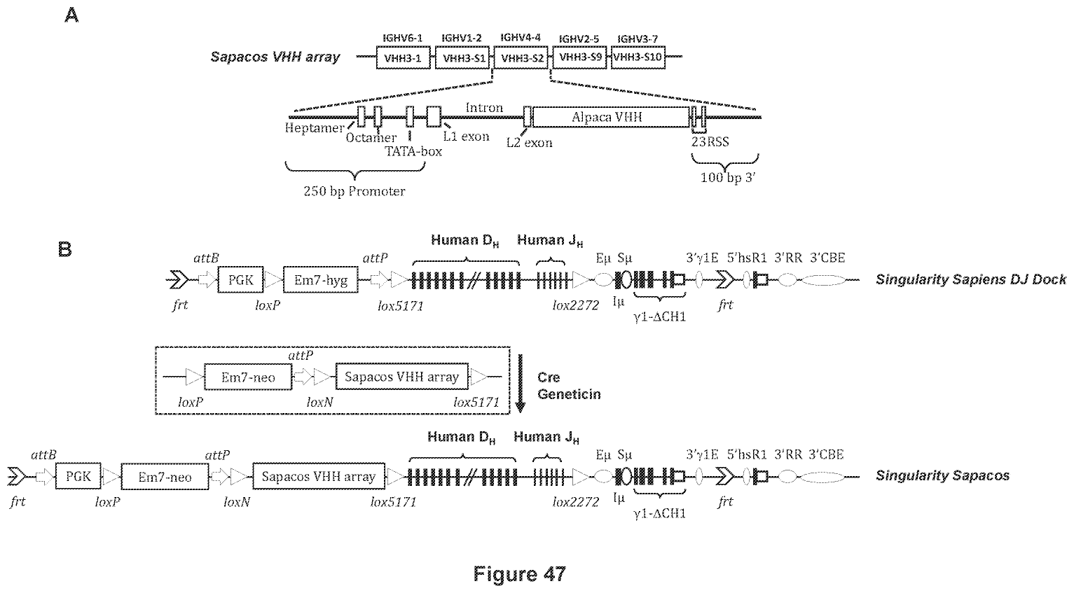

The capability of the Singularity platform was expanded by constructing a synthetic array (Sapacos VHH) containing 5 known VHHs of the alpaca (Vicugna pacos) (Achour et al., J. Immunol., 181(3):2001-2009 (2008)). An individual VHH element was grafted onto the framework of a selected human VH component that included the about 250 bp upstream human promoter containing regulatory elements (e.g., TATA-box, octamer, and heptamer) involved in VH transcription, human leader exon 1 and 2, human intron, and human recombination signal sequences (RSS) (FIG. 47A). Human VHs were selected based on evidence of their high utilization rates in humans and humanized rodent models (e.g., rats and mice). The Sapacos VHH array was designed to contain flanking disparate lox elements to facilitate its targeted integration into the Singularity Sapiens IgH locus via RMCE (FIG. 47B). The Syn Sapacos array (see sequences below) are inserted into the Singularity Sapiens DJ dock allele via RMCE (FIG. 47B). Mice generated from this genetic engineering are called Singularity Sapacos mice and are assessed for their ability to produce alpaca-human-mouse chimeric heavy chain antibodies (e.g., alpaca-human-mouse chimeric heavy chain IgG1-ΔCH1 antibodies).

The alpaca-human-mouse chimeric heavy chain antibodies produced from the Singularity Sapacos mice can have the naturally optimized nanobody properties of alpaca and can access the additional immune diversity of the human D and J elements, enabling rapid humanization for therapeutic applications in humans. The Sapacos VHH array can be readily expanded via repeated rounds of RMCε-mediated integration of arrays containing additional V_{H}Hs from alpaca and other camelid species.

(Alpaca VHH sequence is bold and underline)
1) Human VH: IGHV6-1; Alpaca V_{H}H substitution:
VHH3-1
(SEQ ID NO: 50)
GGGCCCTGCCTCTGAGCTCCTCTTTGCATCCAATCTGCTGAAGAA
CATGGCTCTAGGGAAACCCAGTTGTAGACCTGAGGGCCCCGGCTC
TTCAATGAGCCATCTCCGTCCCGGGGCCTTATATCAGCAAGTGAC
GCACACAGGCAAATGCCAGGGTGTGGTTTCCTGTTTAAATGTAGC
CTCCCCCGCTGCAGAACTGCAGAGCCTGCTGAATTCTGGCTGACC
AGGGCAGTCACCAGAGCTCCAGACAATGTCTGTCTCCTTCCTCAT
CTTCCTGCCCGTGCTGGGCCTCCCATGGGGTCAGTGTCAGGGAGA
TGCCGTATTCACAGCAGCATTCACAGACTGAGGGGTGTTTCACTT
TGCTGTTTCCTTTTGTCTCCAGGTGTCCTGTCACAGGTGCAGCTG
GTGGAGTCAGGTGGAGGATTAGTTCAGGCTGGAGGTTCTCTTCGA
CTATCCTGCGCGGCCAGTGGGCGCACCTTCAGCTCCTATGCCATG
GGCTGGTTTCGTCAAGCACCTGGGAAGGAGAGGGAATTTGTGGCA
GCCATTTCCTGGTCAGGTGGCAGCACATACTATGCAGACTCTGTA
AAAGGGCGGTTTACCATAAGTAGAGACAATGCCAAGAACACTGTG
TACCTGCAGATGAACAGCTTGAAACCAGAAGATACAGCTGTCTAT
TACTGTGCTGCTCACAGTGAGGGGAAGTCAGTGTGAGCCCAGACA
CAAACCTCCCTGCAGGGATGCTCAGGACCCCAGAAGGCACCCAGC
ACTACCAGCGCAGGGCCCAGAC
2) Human VH: IGHV1-2; Alpaca V_{H}H substitution:
VHH3-S1
(SEQ ID NO: 51)
GGGGACACACATCATTAAACAAGGATTGGGACAGGGACTTCAGCG
TCCCACTGTTGCATGGCCCATAAATTATGTGTGTTCTCTTTCTCA
TCTTGGATCAAGTCTAGAGCTATGAAATAGTATCCCTCATGAATA
TGCAAATAACCTGAGATTTACTGAAGTAAATACAGATCTGTCCTG
TGCCCTGAGAGCATCACCCAGCAACCACATCTGTCCTCTAGAGAA
TCCCCTGAGAGCTCCGTTCCTCACCATGGACTGGACCTGGAGGAT
CCTCTTCTTGGTGGCAGCAGCCACAGGTAAGAGGCTCCCTAGTCC
CAGTGATGAGAAAGAGATTGAGTCCAGTCCAGGGAGATCTCATCC
ACTTCTGTGTTCTCTCCACAGGAGCCCACTCCCAGGTGCAGCTGG
TGGAGAGCGGGGGTGGGTTAGTACAACCAGGTGGCTCCCTACGAC
TTAGTTGCGCTGCAAGTGGCAGCATCTTCTCCATAAACGCCATGG
GCTGGTACCGGCAAGCCCCTGGGAAGCAGCGTGAACTGGTGGCAG
CCATCACTTCTGGAGGCTCTACGAACTATGCAGACTCCGTTAAAG
GACGCTTCACCATTTCAAGAGATAATGCCAAGAACACAGTGTATC
TGCAGATGAATTCATTGAAACCCGAGGATACTGCAGTCTACTACT
GTAATGCTCACAGTGTGAAAACCCACATCCTGAGGGTGTCAGAAA
CCCCAGGGAGGAGGCAGCTGTGCTGGGGCTGAGAAATGAAAGGGA
TTACTATTTTTAATGTTG
3) Human VH: IGHV4-4; Alpaca V_{H}H substitution:
VHH3-S2
(SEQ ID NO: 52)
GGGCATGGCTAGTTGAGGCCCCAGGAAGAGAACTGAGTTCTCAAA
GGGCAAAGCAAGCATCCTCATCCCAGGGCGAGCCTAAAAGACTGG
GGCCTCCCTCATCCCTTTTCACCTCTTTATACAAAGGCACCACCT
ACATGCAAATCCTCACTTAGGCACCCACAGGAAACCACCACACAT
TTCCTTAAATTCAGGGTCCAGCTCACATGGGAAATACTTTCTGAG
AGTCATGGACCTCCTGCACAAGAACATGAAACACCTGTGGTTCTT
CCTCCTCCTGGTGGCAGCTCCCAGATGTGAGTGTCTCAAGGCTGC
AGACATGGGGGTATGGGAGGTGCCTCTGATCCCAGGGCTCACTGT
GGGTCTCTCTGTTCACAGGGGTCCTGTCTCAGGTGCAGCTGGTGG
AGTCTGGTGGGGGCCTCGTCCAACCAGGAGGCTCACTCCGACTTT
CCTGTGCTGCTTCAGGATCGATCTTTAGTATAAATGCCATGGGCT
GGTACAGGCAGGCCCCTGGGAAGCAGAGAGAGCTGGTTGCTGCCA
TCAACACTGGAGGTGGCAGCACCTATTATGCAGATTCTGTGAAAG
GGCGTTTTACCATTTCCCGGGACAATGCAAAGAACACACTGTATC
TACAAATGAACAGCTTAAAAAGTGAAGGAACAGCTGTCTACTACT
GCGCAGCCCACAGTGAGGGGAGGTGAGTGTGAGCCCAGACACAAA
CCTCCCTGCAGGGAGGCGGAGGGGACCGGCGCAGGTGCTGCTCAG
AGCCAGCAGGGGGCGCGC
4) Human VH: IGHV2-5; Alpaca V_{H}H substitution:
VHH3-S9
(SEQ ID NO: 53)
TGACTTCTGCAAAGACTTCTACTCAGAATCTACTTGCCCAGCCTT
AGATTAATGCCATCTGAATTACACTGATCATGTTACTATCACTGC
TCCTCACCACAGATGCAACACCCTCCTGAGTCCTGAAACCTGACT
CCATCCCATAGAGTAGGGCACAGATGAGGGGAATGCAAATCTCCA
CCAGCTCCACCCTCCTCTGGGTTGAAAAAGCCGAGCACAGGTCCC
AGCTCAGTGACTCCTGTGCCCCACCATGGACACACTTTGCTCCAC
GCTCCTGCTGCTGACCATCCCTTCATGTGAGTGCTGTGGTCAGGG
ACTCCTTCACGGGTGAAACATCAGTTTTCTTGTTTGTGGGCTTCA
TCTTCTTATGCTTTCTCCACAGGGGTCTTGTCCCAAGTCCAGCTG
GTGGAGTCTGGTGGTGGCCTTGTACAGGCAGGAGGGTCACTTCGG
CACAGCTGTGCTGCGTCTGGACTCACCTTTGGATCCTATGCCATG
GGCTGGTACAGGCAGGCCCCCGGGAAAGAACGAGAGCTGGTTGCT
GCCATCAGCAGTGGGGGCAGTACGTATTATGCAGACTCTGTCAAA
GGCCGCTTCACCATATCCAGAGATAATGCCAAGAACACTCTCTAC
CTGCAGATGAACAGCCTGAAGCCTGAAGGAACAGCTGTGTACTAC
TGCAATGCACACAAAGACACAGCCCAGGGCACCTCCTGTACAAAA
ACCCAGGCTGCTTCTCATTGGTGCTCCCTCCCCACCTCTGCAGAA
CAGGAAAGTGCAGCTGAGA
5) Human VH: IGHV3-7; Alpaca V_{H}H substitution:
VHH3-S10
(SEQ ID NO: 54)
ACAGCATATTTTCCAAATACCATCATTGTCAGCAAACTTCTGCAG
AGCACCGTCTTCTTATATGGGTACAGCCTATTCCTCCAGCATCCC
ACTAGAGCTTCTTATATAGTAGGAGACATGCAAATAGGGCCCTCC
CTCTACTGATGAAAACCAACCCAACCCTGACCCTGCAGGTCTCAG
AGAGGAGCCTTAGCCCTGGACTCCAAGGCCTTTCCACTTGGTGAT
CAGCACTGAGCACAGAGGACTCACCATGGAGTTGGGGCTGAGCTG
GGTTTTCCTTGTTGCTATTTTAGAAGGTGATTCATGGAAAACTAG
GAAGATTGAGTGTGTGTGGATATGAGTGTGAGAAACAGTGGATTT
GTGTGGCAGTTTCTGACCTTGGTGTCTCTTTGTTTGCAGGTGTCC
AGTGTCAGGTGCAGCTGGTGGAGTCAGGAGGTGGTTTGGTTCAAG
CAGGGGGCAGTTTACGACACAGCTGTGCTGCTTCTGGGCTCACTT
TTGGAAGCTATGCCATGGGCTGGTATCGGCAAGCGCCAGGGAAAG
AAAGAGAACTTGTTGCAGCCATAAGCAGTGGTGGCTCAACCTACT
ATGCAGACTCTGTGAAAGGACGCTTCACCATCTCCAGGGATAATG
CCAAGAACACAGTCTACCTGCAGATGAACAGCCTGAAGCCTGAAG
GAACAGCTGTGTCCTACTGCAATGCTCACAGTGAGGGGAAGTCAG
TGTGAGCCCAGACACAAACCTCCCTGCAGGGGTCCCTTGGGACCA
CCAGGGGGCGACAGGGCATTGAGCACGGGGCTGTCT

### Generation of the Singularity Savnars Allelic Series

Cartilaginous fish (e.g., sharks, skates, and rays) produce heavy chain antibodies from a special class of immunoglobulin known as variable new antigen receptor (VNAR) (Greenberg et al., Nature, 374(6518):168-73 (1995)).

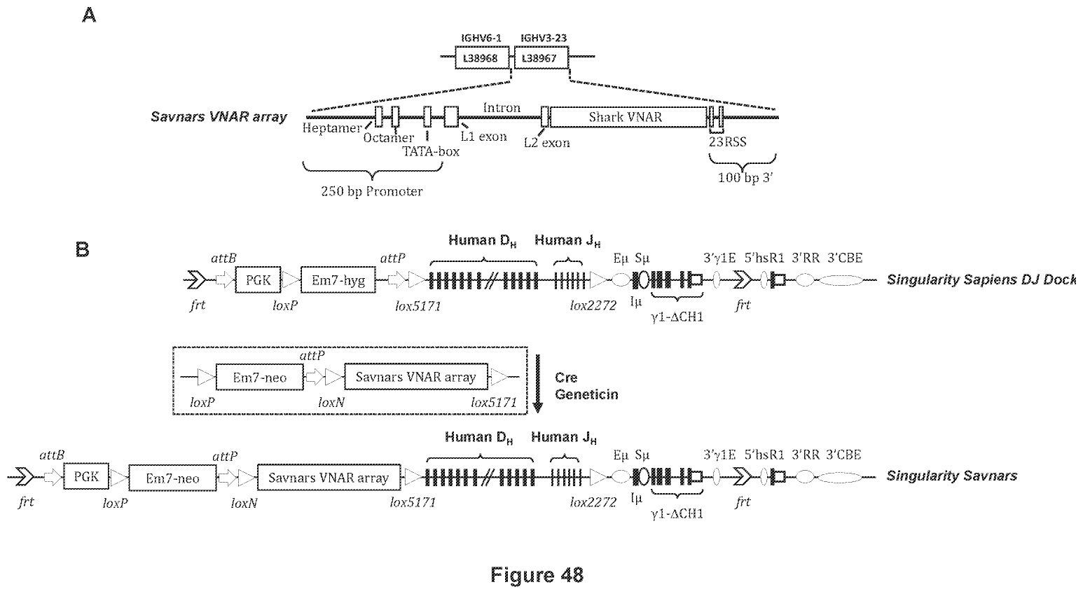

The capability of the Singularity platform was expanded by constructing a synthetic shark VNAR array. The individual VNAR element selected from the germline sequences of the nurse shark Ginglymostoma cirratum was grafted onto the framework of a selected human VH component that included the about 250 bp upstream human promoter containing regulatory elements (e.g., TATA-box, octamer, and heptamer) involved in VH transcription, human leader exon 1 and 2, human intron, and human recombination signal sequences (RSS) (FIG. 48A). The VNAR array dubbed Savnars are synthesized and are inserted into the Singularity Sapiens DJ allele via RMCE (FIG. 48B). Mice that are generated from this genetic engineering are referred to as Singularity Savnars mice and are assessed for the capacity to produce shark-human-mouse chimeric heavy chain antibodies (e.g., shark-human-mouse chimeric heavy chain IgG1-ΔCH1 antibodies).

The shark-human-mouse chimeric heavy chain antibodies produced from the Singularity Savnars mice can have the superior biophysical properties of VNARs and can access the additional immune diversity of the human D and J elements, enabling rapid humanization for therapeutic applications in humans. The immune repertoire of Singularity Savnars mice can be readily expanded via repeated rounds of RMCε-mediated integration of arrays containing additional VNARs from other shark species.

```
Sequence of Singularity Savnars array
(Alpaca V_{H}H sequence is bold and underline)
1) Human VH: IGHV6-1; Nurse shark VNAR
  substitution: L38968
(SEQ ID NO: 55)
GGGCCCTGCCTCTGAGCTCCTCTTTGCATCCAATCTGCTGAAGAA
CATGGCTCTAGGGAAACCCAGTTGTAGACCTGAGGGCCCCGGCTC
TTCAATGAGCCATCTCCGTCCCGGGGCCTTATATCAGCAAGTGAC
GCACACAGGCAAATGCCAGGGTGTGGTTTCCTGTTTAAATGTAGC
CTCCCCCGCTGCAGAACTGCAGAGCCTGCTGAATTCTGGCTGACC
AGGGCAGTCACCAGAGCTCCAGACAATGTCTGTCTCCTTCCTCAT
CTTCCTGCCCGTGCTGGGCCTCCCATGGGGTCAGTGTCAGGGAGA
TGCCGTATTCACAGCAGCATTCACAGACTGAGGGGTGTTTCACTT
TGCTGTTTCCTTTTGTCTCCAGGTGTCCTGTCAGCCAGGGTGGAC
CAGACACCTCGGAGTGTTACCAAGGAGACAGGAGAATCACTCACC
ATCAACTGTGTGCTTCGAGATGCTTCCTATGCATTGGGTTCTACG
TGCTGGTACAGGAAGAAGTCTGGGTCAACAAATGAAGAGAGCATA
AGCAAAGGAGGCAGATATGTGGAGACTGTCAACAGCGGCTCCAAA
AGTTTCTCCCTGAGAATTAATGACCTGACTGTAGAAGATGGTGGC
ACCTACCGCTGTGGGGTCCACAGTGAGGGGAAGTCAGTGTGAGCC
CAGACACAAACCTCCCTGCAGGGATGCTCAGGACCCCAGAAGGCA
CCCAGCACTACCAGCGCAGGGCCCAGAC
2) Human VH: IGHV3-23; Nurse shark
  VNAR substitution: L38967
(SEQ ID NO: 56)
AGCACAATTTCCCAATGCTTTCAATATCACAGATCTCCCCGAGGA
CATTCTGACATGCTCTGAGCCCCACTATCTCCAAAGGCCTCTCAC
CCCAGAGCTTACTATATAGTAGGAGATATGCAAATAGAGCCCTCC
GTCTGCTGATGAAAACCAGCCCAGCCCTGACCCTGCAGCTCTGAG
AGAGGAGCCCAGCCCTGGGATTTTCAGGTGTTTTCATTTGGTGAT
CAGGACTGAACAGAGAGAACTCACCATGGAGTTTGGGCTGAGCTG
GCTTTTTCTTGTGGCTATTTTAAAAGGTAATTCATGGAGAAATAG
AAAAATTGAGTGTGAATGGATAAGAGTGAGAGAAACAGTGGATAC
GTGTGGCAGTTTCTGACCAGGGTTTCTTTTTGTTTGCAGGTGTCC
AGTGTGCTCGAGTAGACCAGACTCCTAAGACTATCACCAAGGAGA
CAGGAGAATCACTCACCATCAACTGTGTTCTTAGAGATACGTCCT
ATGCATTGGGGTCCACCTACTGGTACAGGAAGAAGCTGGGCTCTA
CAAATGAGGAAAGCATTTCAAAAGGTGGGCGGTATGTGGAGACTG
TCAACAGTGGAAGCAAAAGTCTATCTCTGCGTATAAATGGCCTGA
AGGTGGAAGACAGCTGGACATACCGCTGCAAAGCCACAGTGAGGG
GAAGTCATTGTGAGCCCAGACACAAACCTCCCTGCAGGAACGATG
GGGGGGAAATCAGCGGCAGGGGGCGCTCAGGACCCGCTGATCAGA
```

### Example 5—Exemplary Embodiments

Embodiment 1A. A genetically engineered mouse comprising a germline modification comprising a deletion of a nucleic acid sequence comprising one or more heavy-chain C-region genes; wherein the mouse expresses an IgG heavy chain antibody and secretes the IgG heavy chain antibody into its serum.

Embodiment 2A. The genetically engineered mouse of Embodiment 1A, wherein the one or more heavy-chain C-region genes is an IgM C-region gene (Cμ), an IgD C-region gene (Cδ), an IgE C-region gene (Cε), an IgG3 C-region gene (Cγ3), an IgG2b C-region gene (Cγ2b), an IgG2c C-region gene (Cγ2c), or a combination thereof.

Embodiment 3A. The genetically engineered mouse of any one of Embodiments 1A-2A, further comprising a deletion of a nucleic acid sequence encoding a CH1 domain of an IgG1 C-region gene (Cγ1).

Embodiment 4A. The genetically engineered mouse of Embodiment 3A, wherein the deletion of the nucleic acid sequence encoding the CH1 domain of the IgG1 C-region gene comprises exon 1.

Embodiment 5A. The genetically engineered mouse of any one of Embodiments 1A-4A, wherein the germline modification further comprises a native nucleic acid sequence encoding a hinge (H) domain, heavy-chain CH2 domain, a heavy-chain CH3 domain, or a combination thereof.

Embodiment 6A. The genetically engineered mouse of any one of Embodiments 1A-5A, wherein the germline modification further comprises a native nucleic acid sequence comprising an endogenous enhancer.

Embodiment 7A. The genetically engineered mouse of Embodiment 6A, wherein the enhancer is Eμ, 3′RR, 3′γ1E, 5′hsRI, or a combination thereof.

Embodiment 8A. The genetically engineered mouse of any one of Embodiments 1A-7A, wherein the germline modification further comprises a native nucleic acid sequence comprising a switch tandem repeat element (Sμ), wherein Sp drives IgG1 expression.

Embodiment 9A. The genetically engineered mouse of any one of Embodiments 1A-8A, wherein the IgG heavy chain antibody comprises an IgG1 heavy chain antibody.

Embodiment 10A. The genetically engineered mouse of Embodiment 9A, wherein the IgG1 heavy chain antibody is a IgG1ΔCH1 protein.

Embodiment 11A. The genetically engineered mouse of any one of Embodiments 1A-10A, wherein the IgG heavy chain antibody lacks a light chain.

Embodiment 12A. The genetically engineered mouse of any one of Embodiments 1A-11 A, wherein the IgG heavy chain antibody comprises a hinge domain, CH2 domain, a CH3 domain, or a combination thereof.

Embodiment 13A. The genetically engineered mouse of any one of Embodiments 1A-12A, wherein the mouse does not express a wild-type IgM protein, a wild-type IgD protein, a wild-type IgE protein, a wild-type IgG3 protein, or a combination thereof.

Embodiment 14A. The genetically engineered mouse of any one of Embodiments 1A-14A, wherein the mouse does not express a wild-type IgA protein, a wild-type IgG2b protein, a wild-type IgG2c protein, or a combination thereof.

Embodiment 15A. An engineered non-human animal comprising a germline genome comprising an engineered immunoglobulin heavy chain (IgH) allele at an endogenous IgH locus; wherein the engineered IgH allele lacks an endogenous heavy-chain C region gene; and wherein the endogenous heavy-chain C region gene comprises Cμ, Cδ, Cε, Cγ3, Cγ2b, Cγ2c, or a combination thereof.

Embodiment 16A. The engineered non-human animal of Embodiment 15A, wherein the IgH allele comprises a deletion of a nucleic acid sequence encoding a CH1 domain of an IgG1 C-region gene (Cγ1).

Embodiment 17A. The engineered non-human animal of Embodiment 16A, wherein the CH1 domain of the IgG1 C-region gene comprises exon 1.

Embodiment 18A. The engineered non-human animal of any one of Embodiments 15A-17A, wherein the IgH locus comprises a native nucleic acid sequence encoding a hinge (H) domain, heavy-chain CH2 domain, a heavy-chain CH3 domain, or a combination thereof.

Embodiment 19A. The engineered non-human animal of any one of Embodiments 15A-18A, wherein the IgH locus comprises a native nucleic acid sequence comprising an endogenous enhancer.

Embodiment 20A. The engineered non-human animal of Embodiment 19A, wherein the enhancer is Eμ, 3′RR, 3′γ1E, 5′hsRI, or a combination thereof.

Embodiment 21A. The engineered non-human animal of any one of Embodiments 15A-20A, wherein the IgH locus comprises a native nucleic acid sequence comprising a switch tandem repeat element (Sμ), wherein Sp drives IgG1 expression.

Embodiment 22A. The engineered non-human animal of any one of Embodiments 15A-21A, wherein the non-human animal expresses an IgG heavy chain antibody.

Embodiment 23A. The engineered non-human animal of Embodiment 22A, wherein the IgG heavy chain antibody comprises an IgG1 heavy chain antibody.

Embodiment 24A. The engineered non-human animal of any one of Embodiment 22A-23A, wherein the IgG1 heavy chain antibody is a IgG1ΔCH1 protein.

Embodiment 25A. The engineered non-human animal of any one of Embodiments 22A-24A, wherein the IgG heavy chain antibody lacks a light chain.

Embodiment 26A. The engineered non-human animal of any one of Embodiments 22A-25A, wherein the IgG heavy chain antibody comprises a hinge domain, a CH2 domain, a CH3 domain, or a combination thereof.

Embodiment 27A. The engineered non-human animal of any one of Embodiments 15A-26A, wherein the non-human animal does not express wild-type IgM protein, a wild-type IgD protein, a wild-type IgE protein, a wild-type IgG3 protein, or a combination thereof.

Embodiment 28A. The engineered non-human animal of any one of Embodiments 15A-27A, wherein the non-human animal does not express a wild-type IgA protein, a wild-type IgG2b protein, a wild-type IgG2c protein, or a combination thereof.

Embodiment 29A. The engineered non-human animal of any one of Embodiments 15A-28A, wherein the IgH locus comprises endogenous V, D, or J genes.

Embodiment 30A. The engineered non-human animal of any one of Embodiments 15A-29A, wherein the engineered non-human animal is homozygous for the engineered IgH allele.

Embodiment 31A. The engineered non-human animal of any one of Embodiments 15A-30A, wherein the endogenous IgH locus does not comprise an exogenous nucleic acid sequence.

Embodiment 32A. The engineered non-human animal of any one of Embodiments 15A-30A, wherein the endogenous IgH locus comprises an exogenous nucleic acid sequence.

Embodiment 33A. The engineered non-human animal of Embodiment 32A, wherein the exogenous nucleic acid sequence comprises a bar code.

Embodiment 34A. The engineered non-human animal of any one of Embodiments 15A-33A, wherein the non-human animal is a mammal.

Embodiment 35A. The engineered non-human animal of Embodiment 34A, wherein the mammal is a mouse or a rat.

Embodiment 36A. A method of producing a genetically modified non-human animal capable of producing a heavy-chain antibody comprising (a) deleting an endogenous nucleic acid sequence comprising one or more heavy-chain C-region genes from an endogenous immunoglobulin heavy chain locus in a stem cell of a non-human animal; (b) implanting the stem cell into a blastocyst; (c) implanting the blastocyst into a pseudo-pregnant mouse to obtain a chimeric mouse; (d) crossing the chimeric mouse to a wild-type mouse to produce offspring; (e) screening the offspring for heterozygosity; and (f) identifying a founder mouse carrying a deletion of one or more heavy-chain C-region genes; and wherein said non-human animal is capable of producing a heavy chain antibody.

Embodiment 37A. The method of Embodiment 36A, wherein the stem cell is an embryonic stem cell.

Embodiment 38A. The method of any one of Embodiments 36A-37A, wherein the one or more heavy-chain C-region genes comprises Cμ, Cδ, Cγ3, Cγ2b, Cγ2c, Cε, or a combination thereof.

Embodiment 39A. The method of any one of Embodiments 36A-38A, further comprising deleting a nucleic acid sequence encoding a CH1 domain of an IgG1 C-region gene and CH1 exon of Cγ1.

Embodiment 40A. The method of Embodiment 39A, wherein the deletion of the nucleic acid sequence encoding the CH1 domain of the IgG1 C-region gene comprises exon 1.

Embodiment 41A. The method of any one of Embodiments 36A-40A, further comprising preserving a native nucleic acid sequence encoding a hinge (H) domain, heavy-chain CH2 domain, a heavy-chain CH3 domain, or a combination thereof.

Embodiment 42A. The method of any one of Embodiments 36A-41A, further comprising preserving a native nucleic acid sequence comprising an endogenous enhancer.

Embodiment 43A. The method of Embodiment 42A, wherein the enhancer is E p, 3′RR, 3′γ1E, 5′hsRI, or a combination thereof.

Embodiment 44A. The method of any one of Embodiments 36A-43A, further comprising preserving a native nucleic acid sequence comprising a switch tandem repeat element (Sp), wherein Sp drives IgG1 expression.

Embodiment 45A. The method of any one of Embodiments 36A-44A, wherein the heavy chain antibody is an IgG heavy chain antibody.

Embodiment 46A. The method of Embodiment 45A, wherein the IgG heavy chain antibody comprises an IgG1 heavy chain antibody.

Embodiment 47A. The method of Embodiment 46A, wherein the IgG1 heavy chain antibody is a IgG1ΔCH1 protein.

Embodiment 48A. The method of any one of Embodiments 45A-47A, wherein the IgG heavy chain antibody lacks a light chain.

Embodiment 49A. The method of any one of Embodiments 45A-48A, wherein the IgG heavy chain antibody comprises a hinge domain, CH2 domain, a CH3 domain, or a combination thereof.

Embodiment 50A. The method of any one of Embodiments 36A-49A, wherein the non-human animal does not express a wild-type IgM protein, a wild-type IgD protein, a wild-type IgE protein, a wild-type IgG3 protein, or a combination thereof.

Embodiment 51A. The method of any one of Embodiments 36A-50A, wherein the non-human animal does not express a wild-type IgA protein, a wild-type IgG2b protein, a wild-type IgG2c protein, or a combination thereof.

Embodiment 52A. The method of any one of Embodiments 36A-51A, wherein the non-human animal is a mammal.

Embodiment 53A. The method of Embodiment 52A, wherein the mammal is a mouse or a rat.

Embodiment 54A. The method of any one of Embodiments 36A-53A, wherein deleting an endogenous nucleic acid sequence comprising one or more heavy-chain C-region genes comprises CRISPR/Cas9 genome editing.

Embodiment 55A. The method of any one of Embodiments 36A-54A, wherein the genetically modified non-human animal is fertile.

Embodiment 56A. The method of any one of Embodiments 36A-55A, wherein the genetically modified non-human animal has substantially normal B cell development and maturation.

Embodiment 57A. The method of any one of Embodiments 36A-56A, wherein the genetically modified non-human animal does not express a wild-type IgM protein, a wild-type IgD protein, a wild-type IgE protein, a wild-type IgG3 protein, a wild-type IgA protein, a wild-type IgG2b protein, a wild-type IgG2c protein, or a combination thereof.

Embodiment 58A. A method of producing a soluble heavy-chain antibody in the engineered non-human animal of any one of Embodiments 36A-57A comprising (a) administering to the non-human animal an antigen; (b) isolating one or more B cells from the non-human animal; (c) isolating mRNA from the one or more B cells; (d) sequencing the mRNA; (e) identifying clonal type based on the mRNA sequence; and (f) performing phylogenetic analysis of the clonal type; thereby producing a soluble heavy-chain antibody.

Embodiment 59A. The method of Embodiment 58A, wherein the non-human animal is a mammal.

Embodiment 60A. The method of Embodiment 59A, wherein the mammal is a mouse or a rat.

Embodiment 61A. A method of producing a single domain antibody (sdAb) identified from the engineered non-human animal of any one of Embodiments 36A-57A comprising (a) expressing a nucleic acid sequence encoding a heavy chain variable (VH) domain comprising a V, D and J in a cell, wherein the cell produces the heavy chain variable domain; and (b) isolating the heavy chain variable domain from a sample thereby producing the single domain antibody.

Embodiment 62A. The method of Embodiment 61A, wherein the single domain antibody is a murine single domain antibody.

Embodiment 63A. The method of Embodiment 62A, wherein the single domain antibody is an IgG1 single domain antibody.

Embodiment 64A. The method of Embodiment 63A, wherein the IgG1 single domain antibody is a IgG1ΔCH1 nanobody.

Embodiment 65A. The method of any one of Embodiments 61A-64A, wherein the single domain antibody lacks a light chain.

Embodiment 66A. The method of any one of Embodiments 61A-65A, wherein the single domain antibody lacks a hinge domain, a CH2 domain, a CH3 domain or a combination thereof.

Embodiment 1B. A genetically engineered mouse comprising a germline modification comprising a deletion of a nucleic acid sequence comprising one or more heavy-chain C-region genes; wherein the mouse expresses a humanized IgG heavy chain antibody and secretes the humanized IgG heavy chain antibody into its serum.

Embodiment 2B. The genetically engineered mouse of Embodiment 1B, wherein the one or more heavy-chain C-region genes is an IgM C-region gene (Cμ), an IgD C-region gene (Cδ), an IgE C-region gene (Cε), an IgG3 C-region gene (Cγ3), an IgG2b C-region gene (Cγ2b), an IgG2c C-region gene (Cγ2c), or a combination thereof.

Embodiment 3B. The genetically engineered mouse of any one of Embodiments 1B-2B, further comprising a deletion of a nucleic acid sequence encoding a CH1 domain of an IgG1 C-region gene (Cγ1).

Embodiment 4B. The genetically engineered mouse of Embodiment 3B, wherein the deletion of the nucleic acid sequence encoding the CH1 domain of the IgG1 C-region gene comprises exon 1.

Embodiment 5B. The genetically engineered mouse of any one of Embodiments 1B-4B, wherein the germline modification further comprises a native nucleic acid sequence encoding a hinge (H) domain, heavy-chain CH2 domain, a heavy-chain CH3 domain, or a combination thereof.

Embodiment 6B. The genetically engineered mouse of any one of Embodiments 1B-5B, wherein the germline modification further comprises a native nucleic acid sequence comprising an endogenous enhancer.

Embodiment 7B. The genetically engineered mouse of Embodiment 6B, wherein the enhancer is Eμ, 3′RR, 3′γ1E, 5′hsRI, or a combination thereof.

Embodiment 8B. The genetically engineered mouse of any one of Embodiments 1B-7B, wherein the germline modification further comprises a native nucleic acid sequence comprising a switch tandem repeat element (Sμ), wherein Sp drives IgG1 expression.

Embodiment 9B. The genetically engineered mouse of any one of Embodiments 1B-8B, wherein the humanized IgG heavy chain antibody comprises a humanized IgG1 heavy chain antibody.

Embodiment 10B. The genetically engineered mouse of Embodiment 9B, wherein the humanized IgG1 heavy chain antibody is a IgG1ΔCH1 protein.

Embodiment 11B. The genetically engineered mouse of any one of Embodiments 1B-10B, wherein the humanized IgG heavy chain antibody lacks a light chain.

Embodiment 12B. The genetically engineered mouse of any one of Embodiments 1B-11B, wherein the humanized IgG heavy chain antibody comprises a hinge domain, CH2 domain, a CH3 domain, or a combination thereof.

Embodiment 13B. The genetically engineered mouse of any one of Embodiments 1B-12B, wherein the mouse does not express a wild-type IgM protein, a wild-type IgD protein, a wild-type IgE protein, a wild-type IgG3 protein, or a combination thereof.

Embodiment 14B. The genetically engineered mouse of any one of Embodiment 1B-14B, wherein the mouse does not express a wild-type IgA protein, a wild-type IgG2b protein, a wild-type IgG2c protein, or a combination thereof.

Embodiment 15B. An engineered non-human animal comprising a germline genome comprising an engineered immunoglobulin heavy chain (IgH) allele at an endogenous IgH locus; wherein the engineered IgH allele lacks an endogenous heavy-chain C region gene; and wherein the endogenous heavy-chain C region gene comprises Cμ, Cδ, Cε, Cγ3, Cγ2b, Cγ2c, or a combination thereof.

Embodiment 16B. The engineered non-human animal of Embodiment 15B, wherein the IgH allele comprises a deletion of a nucleic acid sequence encoding a CH1 domain of an IgG1 C-region gene (Cγ1).

Embodiment 17B. The engineered non-human animal of Embodiment 16B, wherein the CH1 domain of the IgG1 C-region gene comprises exon 1.

Embodiment 18B. The engineered non-human animal of any one of Embodiments 15B-17B, wherein the IgH locus comprises a native nucleic acid sequence encoding a hinge (H) domain, heavy-chain CH2 domain, a heavy-chain CH3 domain, or a combination thereof.

Embodiment 19B. The engineered non-human animal of any one of Embodiments 15B-18B, wherein the IgH locus comprises a native nucleic acid sequence comprising an endogenous enhancer.

Embodiment 20B. The engineered non-human animal of Embodiment 19B, wherein the enhancer is Eμ, 3′RR, 3′γ1E, 5′hsRI, or a combination thereof.

Embodiment 21B. The engineered non-human animal of any one of Embodiments 15B-20B, wherein the IgH locus comprises a native nucleic acid sequence comprising a switch tandem repeat element (Sμ), wherein Sp drives IgG1 expression.

Embodiment 22B. The engineered non-human animal of any one of Embodiments 15B-21B, wherein the non-human animal expresses a humanized IgG heavy chain antibody.

Embodiment 23B. The engineered non-human animal of Embodiment 22B, wherein the humanized IgG heavy chain antibody comprises a humanized IgG1 heavy chain antibody.

Embodiment 24B. The engineered non-human animal of any one of Embodiments 22B-23B, wherein the humanized IgG1 heavy chain antibody is a IgG1ΔCH1 protein.

Embodiment 25B. The engineered non-human animal of any one of Embodiments 22B-24B, wherein the humanized IgG heavy chain antibody lacks a light chain.

Embodiment 26B. The engineered non-human animal of any one of Embodiments 22B-25B, wherein the humanized IgG heavy chain antibody comprises a hinge domain, a CH2 domain, a CH3 domain, or a combination thereof.

Embodiment 27B. The engineered non-human animal of any one of Embodiments 15B-26B, wherein the non-human animal does not express wild-type IgM protein, a wild-type IgD protein, a wild-type IgE protein, a wild-type IgG3 protein, or a combination thereof.

Embodiment 28B. The engineered non-human animal of any one of Embodiments 15B-27B, wherein the non-human animal does not express a wild-type IgA protein, a wild-type IgG2b protein, a wild-type IgG2c protein, or a combination thereof.

Embodiment 29B. The engineered non-human animal of any one of Embodiments 15B-28B, wherein the IgH locus comprises human V, D, or J genes.

Embodiment 30B. The engineered non-human animal of any one of Embodiments 15B-29B, wherein the engineered non-human animal is homozygous for the engineered IgH allele.

Embodiment 31B. The engineered non-human animal of any one of Embodiments 15B-30B, wherein the endogenous IgH locus comprises an exogenous nucleic acid sequence.

Embodiment 32B. The engineered non-human animal of any one of Embodiments 15B-31B, wherein the exogenous nucleic acid sequence comprises one or more human V_{H} gene segments, one or more human D_{H} gene segments, and one or more J_{H} gene segments.

Embodiment 33B. The engineered non-human animal of any one of Embodiments 15B-32B, wherein the exogenous nucleic acid sequence comprises 65 human V_{H} gene segments.

Embodiment 34B. The engineered non-human animal of any one of Embodiments 15B-32B, wherein the exogenous nucleic acid sequence comprises 27 human D_{H} gene segments.

Embodiment 35B. The engineered non-human animal of any one of Embodiments 15B-32B, wherein the exogenous nucleic acid sequence comprises 6 J_{H} gene segments.

Embodiment 36B. The engineered non-human animal of any one of Embodiments 15B-35B, wherein the exogenous nucleic acid sequence comprises 65 human V_{H} gene segments, 27 human DH gene segments, and 6 J_{H} gene segments.

Embodiment 37B. The engineered non-human animal of Embodiment 31B, wherein the exogenous nucleic acid sequence comprises a bar code.

Embodiment 38B. The engineered non-human animal of any one of Embodiments 15B-37B, wherein the non-human animal is a mammal.

Embodiment 39B. The engineered non-human animal of Embodiment 38B, wherein the mammal is a mouse or a rat.

Embodiment 40B. A method of producing a genetically modified non-human animal capable of producing a humanized heavy-chain antibody comprising (a) deleting an endogenous nucleic acid sequence comprising one or more heavy-chain C-region genes from an endogenous immunoglobulin heavy chain locus in a stem cell of a non-human animal; (b) implanting the stem cell into a blastocyst; (c) implanting the blastocyst into a pseudo-pregnant mouse to obtain a chimeric mouse; (d) crossing the chimeric mouse to a wild-type mouse to produce offspring; (e) screening the offspring for heterozygosity; and (f) identifying a founder mouse carrying a deletion of one or more heavy-chain C-region genes; and wherein said non-human animal is capable of producing a humanized heavy chain antibody.

Embodiment 41B. The method of Embodiment 40B, wherein the stem cell is an embryonic stem cell.

Embodiment 42B. The method of any one of Embodiments 40B-41B, wherein the one or more heavy-chain C-region genes comprises Cμ, Cδ, Cγ3, Cγ2b, Cγ2c, Cε, or a combination thereof.

Embodiment 43B. The method of any one of Embodiments 40B-42B, further comprising deleting a nucleic acid sequence encoding a CH1 domain of an IgG1 C-region gene (Cγ1).

Embodiment 44B. The method of Embodiment 43B, wherein the deletion of the nucleic acid sequence encoding the CH1 domain of the IgG1 C-region gene comprises exon 1.

Embodiment 45B. The method of any one of Embodiments 40B-44B, further comprising preserving a native nucleic acid sequence encoding a hinge (H) domain, heavy-chain CH2 domain, a heavy-chain CH3 domain, or a combination thereof.

Embodiment 46B. The method of any one of Embodiments 40B-45B, further comprising preserving a native nucleic acid sequence comprising an endogenous enhancer.

Embodiment 47B. The method of Embodiment 46B, wherein the enhancer is Eμ, 3′RR, 3′γ1E, 5′hsRI, or a combination thereof.

Embodiment 48B. The method of any one of Embodiments 40B-47B, further comprising preserving a native nucleic acid sequence comprising a switch tandem repeat element (Sp), wherein Sp drives IgG1 expression.

Embodiment 49B. The method of any one of Embodiments 40B-48B, wherein the humanized heavy chain antibody is a humanized IgG heavy chain antibody.

Embodiment 50B. The method of Embodiment 49B, wherein the humanized IgG heavy chain antibody comprises a humanized IgG1 heavy chain antibody.

Embodiment 51B. The method of Embodiment 50B, wherein the IgG1 heavy chain antibody is a IgG1ΔCH1 protein.

Embodiment 52B. The method of any one of Embodiments 50B-51B, wherein the humanized IgG heavy chain antibody lacks a light chain.

Embodiment 53B. The method of any one of Embodiments 49B-52B, wherein the humanized IgG heavy chain antibody comprises a hinge domain, CH2 domain, a CH3 domain, or a combination thereof.

Embodiment 54B. The method of any one of Embodiments 40B-53B, wherein the non-human animal does not express a wild-type IgM protein, a wild-type IgD protein, a wild-type IgE protein, a wild-type IgG3 protein, or a combination thereof.

Embodiment 55B. The method of any one of Embodiments 40B-54B, wherein the non-human animal does not express a wild-type IgA protein, a wild-type IgG2b protein, a wild-type IgG2c protein, or a combination thereof.

Embodiment 56B. The method of Embodiments any one of 40B-55B, wherein the non-human animal is a mammal.

Embodiment 57B. The method of Embodiment 56B, wherein the mammal is a mouse or a rat.

Embodiment 58B. The method of any one of Embodiments 40B-57B, wherein deleting an endogenous nucleic acid sequence comprising one or more heavy-chain C-region genes comprises CRISPR/Cas9 genome editing.

Embodiment 59B. The method of any one of Embodiments 40B-58B, wherein the genetically modified non-human animal is fertile.

Embodiment 60B. The method of any one of Embodiments 40B-59B, wherein the genetically modified non-human animal has substantially normal B cell development and maturation.

Embodiment 61B. The method of any one of Embodiments 40B-60B, wherein the genetically modified non-human animal does not express a wild-type IgM protein, a wild-type IgD protein, a wild-type IgE protein, a wild-type IgG3 protein, a wild-type IgA protein, a wild-type IgG2b protein, a wild-type IgG2c protein, or a combination thereof.

Embodiment 62B. A method of producing a soluble humanized heavy-chain antibody in the engineered non-human animal of any one of Embodiments 40B-61B comprising (a) administering to the non-human animal an antigen; (b) isolating one or more B cells from the non-human animal; (c) isolating mRNA from the one or more B cells; (d) sequencing the mRNA; (e) identifying clonal type based on the mRNA sequence; and (f) performing phylogenetic analysis of the clonal type; thereby producing a soluble humanized heavy-chain antibody.

Embodiment 63B. The method of Embodiment 62B, wherein the non-human animal is a mammal.

Embodiment 64B. The method of Embodiment 63B, wherein the mammal is a mouse or a rat.

Embodiment 65B. A method of producing a humanized single domain antibody (sdAb) identified from the engineered non-human animal of any one of Embodiments 40-61 comprising (a) expressing a nucleic acid sequence encoding a human heavy chain variable (V_{H}) domain comprising a V, a D and a J in a cell, wherein the cell produces the human heavy chain variable domain; and (b) isolating the human heavy chain variable domain from a sample thereby producing the single domain antibody.

Embodiment 66B. The method of Embodiment 65B, wherein the single domain antibody is a human single domain antibody.

Embodiment 67B. The method of Embodiment 66B, wherein the single domain antibody is an IgG1 single domain antibody.

Embodiment 68B. The method of Embodiment 67B, wherein the IgG1 single domain antibody is a IgG1ΔCH1 nanobody.

Embodiment 69B. The method of any one of Embodiments 65B-68B, wherein the single domain antibody lacks a light chain.

Embodiment 70B. The method of any one of Embodiments 65B-69B, wherein the single domain antibody lacks a hinge domain, a CH2 domain, a CH3 domain or a combination thereof.

Embodiment 71B. The method of any one of Embodiments 65B-70B, wherein the cell is a bacterial cell or a human cell.

Embodiment 1C. A DNA comprising a genetically modified non-human immunoglobulin heavy chain (IgH) allele, wherein the genetically modified non-human IgH allele lacks one or more nucleic acid sequences encoding at least a portion of one or more endogenous constant domains comprising a CH1 constant domain of an IgG subclass, an IgM constant domain, an IgD constant domain, an IgE constant domain, an IgA constant domain, or any combination thereof.

Embodiment 2C. The DNA of Embodiment 2C, wherein the DNA is a germline genomic DNA.

Embodiment 3C. The DNA of any one of Embodiments 1C-2C, wherein the genetically modified non-human IgH allele lacks one or more nucleic acid sequences encoding at least a portion of one or more endogenous constant domains comprising a CH1 constant domain of an IgG subclass.

Embodiment 4C. The DNA of Embodiment 3C, wherein the IgG subclass comprises an IgG1, IgG2a, IgG2b, IgG2c, IgG3, or IgG4 subclass.

Embodiment 5C. The DNA of Embodiment 3C, wherein the IgG subclass is the IgG1 subclass.

Embodiment 6C. The DNA of any one of Embodiments 1C-5C, wherein the genetically modified non-human IgH allele comprises a nucleic acid sequence (Cγ1-ΔCH1) encoding a CH1-truncated IgG1 constant domain (IgG1ΔCH1).

Embodiment 7C. The DNA of any one of Embodiments 1C-6C, wherein the genetically modified non-human IgH allele comprises a nucleic acid sequence encoding a hinge (H) domain, a CH2 domain, a CH3 domain of the IgG subclass, or any combination thereof.

Embodiment 8C. The DNA of any one of Embodiments 1C-7C, wherein the genetically modified non-human IgH allele lacks one or more nucleic acid sequences encoding at least a portion of one or more endogenous constant domains comprising an IgG2 constant domain, IgG3 constant domain, IgG4 constant domain, or any combination thereof.

Embodiment 9C. The DNA of any one of Embodiments 1C-8C, wherein the genetically modified non-human IgH allele comprises one or more endogenous enhancers comprising Ep, 3′γ1E, 5′hsR1, 3′RR, or any combination thereof.

Embodiment 10C. The DNA of any one of Embodiments 1C-9C, wherein the genetically modified non-human IgH allele comprises an Iμ promoter, an Iμ exon, or both.

Embodiment 11 C. The DNA of any one of Embodiments 1C-10C, wherein the genetically modified non-human IgH allele comprises a switch tandem repeat element (Sμ).

Embodiment 12C. The DNA of any one of Embodiments 1C-I1C, wherein IgG1 expression is driven by the Eμ, Iμ promoter, Sμ, or any combination thereof.

Embodiment 13C. The DNA of any one of Embodiments 1C-12C, wherein the genetically modified non-human IgH allele lacks one or more endogenous switch regions comprising Sγ3, Sγ1, Sγ2b, Sγ2c, Sε, Sα, or any combination thereof.

Embodiment 14C. The DNA of any one of Embodiments 1C-13C, wherein the genetically modified non-human IgH allele comprises the following components (from 5′ to 3′): Eμ, Iμ promoter, Iμ exon, Sμ, Cγ1-ΔCH1, 3′γ1E, 5′hsR1, and 3′RR.

Embodiment 15C. The DNA of any one of Embodiments 1C-14C, wherein the genetically modified non-human IgH allele comprises a flippase recognition target (frt) site.

Embodiment 16C. The DNA of any one of Embodiments 1C-15C, wherein the genetically modified non-human IgH allele comprises endogenous V gene segments, D gene segments, J gene segments, or any combination thereof.

Embodiment 17C. The DNA of any one of Embodiments 1C-16C, wherein the genetically modified non-human IgH allele lacks at least one endogenous V gene segments, D gene segments, J gene segments, or any combination thereof.

Embodiment 18C. The DNA of any one of Embodiments 1C-17C, wherein the genetically modified non-human IgH allele comprises a docking cassette.

Embodiment 19C. The DNA of Embodiment 18C, wherein the docking cassette comprises a left and right homology arm, an frt site, an attB site, a promoter, a loxP site, a nucleic acid sequence encoding a selection marker, or any combination thereof.

Embodiment 20C. The DNA of Embodiment 18C, wherein the docking cassette comprises a nucleic acid sequence encoding a selection marker.

Embodiment 21C. The DNA of Embodiment 20C, wherein the selection marker comprises geneticin, hydromycin, puromycin, or any combination thereof.

Embodiment 22C. The DNA of any one of Embodiments 1C-21C, wherein the genetically modified non-human IgH allele encodes an IgG heavy chain antibody.

Embodiment 23C. The DNA of any one of Embodiments 1C-22C, wherein the genetically modified non-human IgH allele comprises exogenous V gene segments, exogenous D gene segments, exogenous J gene segments, or any combination thereof.

Embodiment 24C. The DNA of Embodiment 23C, wherein the exogenous gene segments are selected from the group consisting of human, mouse, rat, bovine, alpaca, and shark gene segments.

Embodiment 25C. The DNA of Embodiment 23C, wherein the exogenous gene segments comprise human gene segments.

Embodiment 26C. The DNA of any one of Embodiments 1C-25C, wherein the genetically modified non-human IgH allele comprises one or more human VH gene segments, one or more human DH gene segments, and one or more human JH gene segments.

Embodiment 27C. The DNA of any one of Embodiments 1C-26C, wherein the genetically modified non-human IgH allele comprises at least 10, 20, 30, 40, 50, 60, 80, 100, 120, or 126 human VH gene segments.

Embodiment 28C. The DNA of any one of Embodiments 1C-27C, wherein the genetically modified non-human IgH allele comprises at least 10, 15, 20, 25, or 27 human DH gene segments.

Embodiment 29C. The DNA of any one of Embodiments 1C-28C, wherein the genetically modified non-human IgH allele comprises at least 1, 2, 3, 4, 5, 6, 7, 8, or 9 human JH gene segments.

Embodiment 30C. The DNA of any one of Embodiments 1C-29C, wherein the genetically modified non-human IgH allele comprises 126 human VH gene segments, 27 human DH gene segments, and 9 human JH gene segments.

Embodiment 31C. The DNA of any one of Embodiments 1C-30C, wherein the genetically modified non-human IgH allele comprises one or more bovine gene segments.

Embodiment 32C. The DNA of Embodiment 31C, wherein the one or more bovine gene segments comprise an L1 exon, an L2 exon of IGHV1-7, a coding segment of IGHD8-2, a coding sequence of IGHJ2-4, a IGH2-4 splice donor, or any combination thereof.

Embodiment 33C. The DNA of any one of Embodiments 31C-32C, wherein the one or more bovine gene segments comprise IGHD4-1, IGHD5-3, IGHD8-2, IGHD1-3, IGHD7-3, IGHD7-4, IGHD6-3, IGHD3-3, or any combination thereof.

Embodiment 34C. The DNA of any one of Embodiments 31C-33C, wherein the one or more bovine gene segments comprise a nucleic acid sequence selected from SEQ ID NOs:42-49 and 57.

Embodiment 35C. The DNA of any one of Embodiments 32C-34C, wherein the DNA comprises one or more human VH gene segments.

Embodiment 36C. The DNA of any one of Embodiments 32C-35C, wherein the DNA comprises one or more human JH gene segments.

Embodiment 37C. The DNA of any one of Embodiments 1C-36C, wherein the genetically modified non-human IgH allele comprises one or more alpaca gene segments.

Embodiment 38C. The DNA of Embodiment 37C, wherein one or more alpaca gene segments comprise VHH3-1, VHH3-S1, VHH3-S2, VHH3-S9, VHH3-S10, or any combination thereof.

Embodiment 39C. The DNA of any one of Embodiments 37C-38C, wherein the alpaca gene segments comprise a nucleic acid sequence selected from SEQ ID NOs:50-54.

Embodiment 40C. The DNA of any one of Embodiments 37C-39C, wherein the DNA comprises one or more human VH gene segments.

Embodiment 41C. The DNA of any one of Embodiments 37C-40C, wherein the DNA comprises one or more human JH gene segments.

Embodiment 42C. The DNA of any one of Embodiments 1C-41C, wherein the genetically modified non-human IgH allele comprises one or more shark gene segments.

Embodiment 43C. The DNA of Embodiment 42C, wherein one or more shark gene segments comprise VNAR-L38968, VNAR-L38967, or both.

Embodiment 44C. The DNA of any one of Embodiments 42C-43C, wherein the shark gene segments comprise a nucleic acid sequence selected from SEQ ID NOs:55-56.

Embodiment 45C. The DNA of any one of Embodiments 42C-44C, wherein the DNA comprises one or more human VH gene segments.

Embodiment 46C. The DNA of any one of Embodiments 42C-45C, wherein the DNA comprises one or more human JH gene segments.

Embodiment 47C. The DNA of any one of Embodiments 1C-46C, wherein the genetically modified non-human IgH allele encodes an IgG heavy chain antibody, and wherein the IgG heavy chain antibody comprises a kappa light chain variable domain, a lambda light chain variable domain, or both.

Embodiment 48C. The DNA of Embodiment 47C, wherein the genetically modified non-human IgH allele comprises one or more exogenous human lambda light chain (LV) gene segments.

Embodiment 49C. The DNA of Embodiment 48C, wherein the one or more human LV gene segments comprise CH17-262M19, CH17-329P5, CH17-238D3, CH17-261A15, CH17-264L24, CH17-117C7, RP11-1040J16, CH17-320F4, or any combination thereof.

Embodiment 50C. The DNA of any one of Embodiments 47C-49C, wherein the genetically modified non-human IgH allele comprises one or more exogenous human kappa light chain (KV) gene segments.

Embodiment 51C. The DNA of Embodiment 50C, wherein the one or more human KV gene segments comprise CH17-272M2, CH17-405H5, CH17-140P2, CH17-13E7, CH17-84J8, CH17-53L15, or any combination thereof.

Embodiment 52C. The DNA of any one of Embodiments 47C-51C, wherein the DNA comprises one or more human VH gene segments.

Embodiment 53C. The DNA of any one of Embodiments 47C-52C, wherein the DNA comprises one or more human JH gene segments.

Embodiment 54C. A genetically modified cell comprising the DNA of any one of Embodiments 1C-53C.

Embodiment 55C. The cell of Embodiment 54C, wherein the cell is a non-human animal cell.

Embodiment 56C. The cell of Embodiment 54C, wherein the cell is a mammalian cell.

Embodiment 57C. The cell of Embodiment 56C, wherein the mammalian cell is a mouse, rat, bovine, alpaca, cat, dog, rabbit, pig, monkey, or chimpanzee cell.

Embodiment 58C. The cell of Embodiment 54C, wherein the cell is a mouse cell.

Embodiment 59C. The cell of Embodiment 54C, wherein the cell is a shark cell.

Embodiment 60C. The cell of Embodiment 54C, wherein the cell is a human cell.

Embodiment 61C. The cell of any one of Embodiments 54C-60C, wherein the cell is a stem cell.

Embodiment 62C. The cell of Embodiment 61C, wherein the stem cell is an embryonic stem cell (ESC) or induced pluripotent stem cells (iPSCs).

Embodiment 63C. The cell of any one of Embodiments 54C-60C, wherein the cell is a B cell.

Embodiment 64C. A genetically modified non-human animal, wherein the genetically modified non-human animal comprises a cell of any one of Embodiments 54C-63C.

Embodiment 65C. The genetically modified non-human animal of Embodiment 64C, wherein the non-human animal is mammal.

Embodiment 66C. The genetically modified non-human animal of Embodiment 65C, wherein the mammal is a mouse, rat, bovine, alpaca, cat, dog, rabbit, pig, monkey, or chimpanzee.

Embodiment 67C. The genetically modified non-human animal of Embodiment 64C, wherein the non-human animal is a mouse.

Embodiment 68C. The genetically modified non-human animal of any one of Embodiments 64C-67C, wherein the genetically modified non-human animal comprises a cell expressing an IgG heavy chain antibody.

Embodiment 69C. The genetically modified non-human animal of Embodiment 68C, wherein the IgG heavy chain antibody is secreted into the serum of the genetically modified non-human animal.

Embodiment 70C. The genetically modified non-human animal of any one of Embodiments 68C-69C, wherein the IgG heavy chain antibody is a CH1-truncated IgG1 heavy chain antibody (IgG1ΔCH1).

Embodiment 71C. The genetically modified non-human animal of any one of Embodiments 68C-70C, wherein the IgG heavy chain antibody lacks a light chain.

Embodiment 72C. The genetically modified non-human animal of any one of Embodiments 68C-71C, wherein the IgG heavy chain antibody comprises a hinge domain, CH2 domain, a CH3 domain, or any combination thereof.

Embodiment 73C. The genetically modified non-human animal of any one of Embodiments 68C-72C, wherein the cell expressing the IgG heavy chain antibody does not express an IgM antibody, an IgD antibody, an IgE antibody, an IgG3 antibody, an IgG2b antibody, an IgG2c antibody, an IgA antibody, or any combination thereof.

Embodiment 74C. The genetically modified non-human animal of any one of Embodiments 68C-73C, wherein the IgG heavy chain antibody is a human IgG heavy chain antibody.

Embodiment 75C. The genetically modified non-human animal of any one of Embodiments 68C-74C, wherein the IgG heavy chain antibody comprises an exogenous variable domain selected from the group consisting of human, mouse, rat, bovine, alpaca, and shark variable domains.

Embodiment 76C. The genetically modified non-human animal of any one of Embodiments 68C-75C, wherein the IgG heavy chain antibody comprises a kappa light chain variable domain, a lambda light chain variable domain, or both.

Embodiment 77C. A method for preparing a germline genomic DNA, wherein the method comprises deleting one or more nucleic acid sequences from a non-human immunoglobulin heavy chain (IgH) allele, wherein the deleted one or more nucleic acid sequences encode at least a portion of one or more endogenous constant domains comprising a CH1 constant domain of an IgG subclass, an IgM constant domain, an IgD constant domain, an IgE constant domain, an IgA constant domain, or any combination thereof, thereby generating a genetically modified non-human IgH allele in the germline genomic DNA.

Embodiment 78C. The method of Embodiment 77C, wherein the germline genomic DNA comprises the DNA of any one of Embodiments 1C-53C.

Embodiment 79C. The method of any one of Embodiments 77C-78C, wherein the IgG constant domain comprises a constant domain of an IgG subclass.

Embodiment 80C. The method of Embodiment 79C, wherein the IgG subclass comprises an IgG1, IgG2a, IgG2b, IgG2c, IgG3, or IgG4 subclass.

Embodiment 81C. A method for preparing a genetically modified non-human animal, wherein the method comprises:
(a) deleting one or more nucleic acid sequences from a non-human immunoglobulin heavy chain (IgH) allele, wherein the deleted one or more nucleic acid sequences encode at least a portion of one or more endogenous constant domains comprising a CH1 constant domain of an IgG subclass, an IgM constant domain, an IgD constant domain, an IgE constant domain, an IgA constant domain, or any combination thereof, thereby generating a genetically modified non-human IgH allele in a germline genomic DNA;
(b) implanting a cell comprising the germline genomic DNA into a blastocyst;
(c) implanting the blastocyst into a pseudo-pregnant non-human animal to obtain a chimeric non-human animal;
(d) crossing the chimeric non-human animal to a wild-type non-human animal to produce offspring;
(e) screening the offspring for heterozygosity; and
(f) identifying the genetically modified non-human animal carrying the deletion of the one or more nucleic acid sequences and capable of producing a heavy chain antibody.

Embodiment 82C. The method of Embodiment 81C, wherein the genetically modified non-human animal is the genetically modified non-human animal of any one of Embodiments 64C-76C.

Embodiment 83C. The method of any one of Embodiments 81C-82C, wherein deleting the one or more nucleic acid sequences comprises using a CRISPR/Cas genome editing system.

Embodiment 84C. The method of Embodiment 83C, wherein the CRISPR/Cas genome editing system comprises at least one guide RNA (gRNA) targeting an endogenous heavy-chain C region gene and a Cas protein.

Embodiment 85C. The method of any one of Embodiments 83C-84C, wherein the Cas protein comprises a Cas9 protein.

Embodiment 86C. The method of any one of Embodiments 81C-85C, wherein the deleted one or more nucleic acid sequences encode the CH1 constant domain of IgG1, the IgG3 constant domain, the IgM constant domain, and the IgD constant domain.

Embodiment 87C. The method of any one of Embodiments 81C-86C, wherein the deleted one or more nucleic acid sequences encode the IgG2 constant domain and the IgA constant domain.

Embodiment 88C. The method of any one of Embodiments 81C-87C, wherein deleting the nucleic acid sequence comprises removing a selection marker from the non-human IgH allele using transient expression of Flp recombinase.

Embodiment 89C. The method of any one of Embodiments 81C-88C, wherein the deleted one or more nucleic acid sequences encode the CH1 constant domain of the IgG subclass, the IgM constant domain, the IgD constant domain, the IgE constant domain, and the IgA constant domain.

Embodiment 90C. The method of any one of Embodiments 81C-89C, wherein the method comprises deleting a nucleic acid sequence from the non-human IgH allele, wherein the nucleic acid sequence comprises endogenous V gene segments, D gene segments, J gene segments or any combination thereof.

Embodiment 91C. The method of any one of Embodiments 81C-90C, wherein the method comprises inserting a docking cassette.

Embodiment 92C. The method of Embodiment 91C, wherein the method comprises contacting the docking cassette with a bacterial artificial chromosome (BAC), wherein the BAC comprises a nucleic acid sequence comprising exogenous V_{H}, D_{H}, and J_{H} gene segments.

Embodiment 93C. The method of Embodiments 92C, wherein the method comprises inserting the exogenous gene segments into the docketing cassette.

Embodiment 94C. The method of any one of Embodiments 92C-93C, wherein the exogenous gene segments are human gene segments.

Embodiment 95C. A genetically modified non-human animal, wherein the genetically modified non-human animal is prepared using the method of any one of Embodiments 81C-94C.

Embodiment 96C. A method of producing an IgG heavy-chain antibody in a genetically modified non-human animal, wherein the method comprises:
(a) administering an antigen to the genetically modified non-human animal of any one of Embodiments 64C-76C;
(b) isolating one or more B cells from the genetically modified non-human animal;
(c) isolating mRNA from the one or more B cells; and
(d) producing the IgG heavy-chain antibody.

Embodiment 97C. The method of Embodiment 96C, wherein the genetically modified non-human animal comprises the DNA of any one of Embodiments 1C-53C.

Embodiment 98C. The method of any one of Embodiments 96C-97C, wherein the method comprises sequencing the mRNA isolated from the one or more B cells.

Embodiment 99C. The method of any one of Embodiments 96C-98C, wherein the method comprises identifying a clonal type based on the mRNA sequence.

Embodiment 100C. The method of Embodiment 99C, wherein the method comprises performing a phylogenetic analysis of the clonal type.

Embodiment 101C. The method of any one of Embodiments 96C-100C, wherein the IgG heavy-chain antibody is a humanized IgG heavy-chain antibody.

Embodiment 102C. The method of any one of Embodiments 96C-100C, wherein the IgG heavy-chain antibody is a IgG heavy-chain antibody comprising a human variable region and a non-human constant region.

Embodiment 103C. An IgG heavy chain antibody, wherein the IgG heavy chain antibody is produced by the method of any one of Embodiments 96C-102C.

Embodiment 104C. A recombinant vector system comprising at least one nucleic acid construct encoding a CRISPR/Cas genome editing system comprising a Cas protein and at least one guide RNA (gRNA), wherein the Cas protein and at least one gRNA form a complex that deletes one or more nucleic acid sequences from a non-human immunoglobulin heavy chain (IgH) allele, wherein the deleted one or more nucleic acid sequences encode at least a portion of one or more endogenous constant domains comprising a CH1 constant domain of an IgG subclass, an IgM constant domain, an IgD constant domain, an IgE constant domain, an IgA constant domain, or any combination thereof.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

All references, publications, patents, and patent applications mentioned in this specification are herein incorporated by reference to the same extent as if each individual publication, patent, or patent application was specifically and individually indicated to be incorporated by reference.

While this disclosure has been particularly shown and described with references to example embodiments thereof, it will be understood by those skilled in the art that various changes in form and details may be made therein without departing from the scope of the invention encompassed by the appended claims.

1-28. (canceled)

29. An engineered non-human animal comprising a germline genome comprising an engineered immunoglobulin heavy chain (IgH) allele at an endogenous IgH locus; wherein the engineered IgH allele lacks an endogenous heavy-chain C region gene; and wherein the endogenous heavy-chain C region gene comprises Cμ, Cδ, Cε, Cγ3, Cγ2b, Cγ2c, or a combination thereof.

30. The engineered non-human animal of claim 29, wherein the IgH allele comprises a deletion of a nucleic acid sequence encoding a CH1 domain of an IgG1 C-region gene (Cγ1).

31-35. (canceled)

36. The engineered non-human animal of claim 29, wherein the IgH locus comprises endogenous V, D, or J genes.

37-41. (canceled)

42. The engineered non-human animal of claim 29, wherein the IgH locus comprises human V, D, or J genes.

43-47. (canceled)

48. The engineered non-human animal of claim 29, wherein the exogenous nucleic acid sequence comprises 65 human V_{H} gene segments, 27 human D_{H} gene segments, and 6 J_{H} gene segments.

49. (canceled)

50. The engineered non-human animal of claim 29, wherein the non-human animal does not express wild-type IgM protein, a wild-type IgD protein, a wild-type IgE protein, a wild-type IgG3 protein, or a combination thereof.

51-54. (canceled)

55. A method of producing a genetically modified non-human animal capable of producing a heavy-chain antibody comprising (a) deleting an endogenous nucleic acid sequence comprising one or more heavy-chain C-region genes from an endogenous immunoglobulin heavy chain locus in a stem cell of a non-human animal; (b) implanting the stem cell into a blastocyst; (c) implanting the blastocyst into a pseudo-pregnant non-human animal to obtain a chimeric non-human animal; (d) crossing the chimeric non-human to a wild-type non-human to produce offspring; (e) screening the offspring for heterozygosity; and (f) identifying a founder non-human carrying a deletion of one or more heavy-chain C-region genes; and wherein said non-human animal is capable of producing a heavy chain antibody.

56-58. (canceled)

59. A method of producing a single domain antibody (sdAb) identified from the engineered non-human animal of claim 55 comprising (a) expressing a nucleic acid sequence encoding a heavy chain variable (V_{H}) domain comprising a V, D and J in a cell, wherein the cell produces the heavy chain variable domain; and (b) isolating the heavy chain variable domain from a sample thereby producing the single domain antibody.

60-64. (canceled)

65. A non-human animal, wherein the genome of said non-human animal comprises an immunoglobulin heavy chain (IgH) allele, wherein said IgH allele comprises an endogenous nucleic acid encoding a CH2 or CH3 domain of an IgG subclass, wherein said IgH allele lacks nucleic acid encoding at least a portion of an endogenous CH1 domain of said IgG subclass, and wherein said IgH allele lacks endogenous nucleic acid encoding at least a portion of an IgM constant domain, endogenous nucleic acid encoding at least a portion of an IgD constant domain, endogenous nucleic acid encoding at least a portion of an IgE constant domain, or endogenous nucleic acid encoding at least a portion of an IgA constant domain.

66. The non-human animal of claim 65, wherein said IgH allele of said non-human animal comprises endogenous nucleic acid encoding said CH2 domain and said CH3 domain of said IgG subclass.

67. (canceled)

68. The non-human animal of claim 65, wherein said IgG subclass is an IgG2 subclass.

69-76. (canceled)

77. The non-human animal of claim 65, wherein said IgH allele lacks endogenous nucleic acid encoding each of the IgM constant domains.

78. The non-human animal of claim 65, wherein said IgH allele lacks endogenous nucleic acid encoding each of the IgD constant domains.

79. The non-human animal of claim 65, wherein said IgH allele lacks endogenous nucleic acid encoding each of the IgE constant domains.

80. The non-human animal of claim 65, wherein said IgH allele lacks endogenous nucleic acid encoding IgA CH1 and CH2 constant domains.

81. The non-human animal of claim 65, wherein said IgH allele lacks nucleic acid encoding said endogenous CH1 domain.

82-100. (canceled)

101. The non-human animal of claim 65, wherein both alleles of said genome lack all the exons of the endogenous Ig heavy chain variable region.

102-113. (canceled)

114. The non-human animal of claim 65, wherein said genome comprises 126 human Ig VH gene segments, 27 or more human Ig VD gene segments, and 9 human Ig VJ gene segments.

115. The non-human animal of claim 65, wherein said non-human animal produces human-non-human chimeric Ig heavy chain antibodies.

116-131. (canceled)

132. The non-human animal of claim 65, wherein said non-human animal is of a first non-human species, and wherein said IgH allele comprises exogenous nucleic acid encoding one or more Ig heavy chain variable region gene segments of a second non-human species that is different from said first non-human species.

133-156. (canceled)


SEQUENCE LISTING (57 sequences)

SEQ ID NO: 1 - protein, 12 aa, Artificial
  CDR of a heavy chain antibody having the ability to bind to a SARS-CoV2 Spike antigen
YTFTDYYMKWLK

SEQ ID NO: 2 - protein, 12 aa, Artificial
  CDR of a heavy chain antibody having the ability to bind to a SARS-CoV2 Spike antigen
YTFTDYYVKWEK

SEQ ID NO: 3 - protein, 12 aa, Artificial
  CDR of a heavy chain antibody having the ability to bind to a SARS-CoV2 Spike antigen
YTFTDYYVKWKK

SEQ ID NO: 4 - protein, 12 aa, Artificial
  CDR of a heavy chain antibody having the ability to bind to a SARS-CoV2 Spike antigen
YTFTDYYIKWEK

SEQ ID NO: 5 - protein, 12 aa, Artificial
  CDR of a heavy chain antibody having the ability to bind to a SARS-CoV2 Spike antigen
YTFTDYYMKWVK

SEQ ID NO: 6 - protein, 12 aa, Artificial
  CDR of a heavy chain antibody having the ability to bind to a SARS-CoV2 Spike antigen
YTFTDYYMKWAK

SEQ ID NO: 7 - protein, 12 aa, Artificial
  CDR of a heavy chain antibody having the ability to bind to a SARS-CoV2 Spike antigen
YTFTDYYMKWEK

SEQ ID NO: 8 - protein, 10 aa, Artificial
  CDR of a heavy chain antibody having the ability to bind to a SARS-CoV2 Spike antigen
GDTFYNQKFK

SEQ ID NO: 9 - protein, 10 aa, Artificial
  CDR of a heavy chain antibody having the ability to bind to a SARS-CoV2 Spike antigen
GDIFYNPQFK

SEQ ID NO: 10 - protein, 10 aa, Artificial
  CDR of a heavy chain antibody having the ability to bind to a SARS-CoV2 Spike antigen
GETFYNQQFK

SEQ ID NO: 11 - protein, 10 aa, Artificial
  CDR of a heavy chain antibody having the ability to bind to a SARS-CoV2 Spike antigen
GDTNYSQNFK

SEQ ID NO: 12 - protein, 10 aa, Artificial
  CDR of a heavy chain antibody having the ability to bind to a SARS-CoV2 Spike antigen
GDTSYNQKFK

SEQ ID NO: 13 - protein, 10 aa, Artificial
  CDR of a heavy chain antibody having the ability to bind to a SARS-CoV2 Spike antigen
GGARYNQKFK

SEQ ID NO: 14 - protein, 10 aa, Artificial
  CDR of a heavy chain antibody having the ability to bind to a SARS-CoV2 Spike antigen
GGTRYNQKFR

SEQ ID NO: 15 - protein, 10 aa, Artificial
  CDR of a heavy chain antibody having the ability to bind to a SARS-CoV2 Spike antigen
GGTNYNQKFK

SEQ ID NO: 16 - protein, 16 aa, Artificial
  CDR of a heavy chain antibody having the ability to bind to a SARS-CoV2 Spike antigen
CARKEYGDYGYATDYW

SEQ ID NO: 17 - protein, 16 aa, Artificial
  CDR of a heavy chain antibody having the ability to bind to a SARS-CoV2 Spike antigen
CARKEYGNYGYAVDYW

SEQ ID NO: 18 - protein, 16 aa, Artificial
  CDR of a heavy chain antibody having the ability to bind to a SARS-CoV2 Spike antigen
CARKEYGNFGYAVDYW

SEQ ID NO: 19 - protein, 16 aa, Artificial
  CDR of a heavy chain antibody having the ability to bind to a SARS-CoV2 Spike antigen
CARKEYGDYGYAVDYW

SEQ ID NO: 20 - protein, 16 aa, Artificial
  CDR of a heavy chain antibody having the ability to bind to a SARS-CoV2 Spike antigen
CARLEDGYYGYAVDYW

SEQ ID NO: 21 - protein, 16 aa, Artificial
  CDR of a heavy chain antibody having the ability to bind to a SARS-CoV2 Spike antigen
CARLEDGYYGYTMDYW

SEQ ID NO: 22 - protein, 16 aa, Artificial
  CDR of a heavy chain antibody having the ability to bind to a SARS-CoV2 Spike antigen
CSRLEDGYYGYAVDYW

SEQ ID NO: 23 - protein, 16 aa, Artificial
  CDR of a heavy chain antibody having the ability to bind to a SARS-CoV2 Spike antigen
CARLEDDYYGYAVDYW

SEQ ID NO: 24 - protein, 16 aa, Artificial
  CDR of a heavy chain antibody having the ability to bind to a SARS-CoV2 Spike antigen
CARLEDGYYGYAIDYW

SEQ ID NO: 25 - protein, 111 aa, Artificial
  nanobody clone
EVQLQQSGPELVKPGASVKISCKASGYTFTDYYMNWVKQSHGKSLEWIGDINPNNGGTSYNQKFKGKATLTVDKSSSTAYMELRSLTSEDSAVYYCARDYWGQGTSVTVSS

SEQ ID NO: 26 - protein, 121 aa, Artificial
  nanobody clone
EVQLQQSGPELVKPGASVKMSCKASGYTFTDYYMKWLKQNHGKSLEWIGDINPNNGDTFYNQKFKGKATLTVDTSSSTAYMQLNSLTSEDSAVYYCARKEYGDYGYATDYWGQGTSVTVSS

SEQ ID NO: 27 - protein, 121 aa, Artificial
  nanobody clone
EVQLQQSGPELVKPGASVKMSCKASGYTFTDYYVKWEKQSHGESLEWIGDINPNNGDTFYNQKFKDKATLTVDKSSSTAYMQLNSLTSEDSAVYYCARKEYGNYGYAVDYWGQGTSVTVSS

SEQ ID NO: 28 - protein, 121 aa, Artificial
  nanobody clone
EVQLQQSGPELVKPGASVKMSCKASGYTFTDYYVKWKKQSHGKSLEWIGDINPNNGDTFYNQKFKDKATLTVDKSSSTAYMQLNSLTSEDSAVYYCARKEYGNFGYAVDYWGQGTSVTVSS

SEQ ID NO: 29 - protein, 121 aa, Artificial
  nanobody clone
EVQLQQSGPELVKPGASVKMSCKASGYTFTDYYIKWEKQSHGKSLEWIGDINPNNGDIFYNPQFKGRATLTVDKSSSTAYMQLNSLTSEDSAVYYCARKEYGDYGYAVDYWGQGTSVTVSS

SEQ ID NO: 30 - protein, 121 aa, Artificial
  nanobody clone
EVQLQQSGPELVKPGASVKMSCKASGYTFTDYYIKWEKQSHGKSLEWIGDINPKNGETFYNQQFKGKATLTVDKSSSTAYMQLNSLTSEDSAVYYCARKEYGDYGYAVDYWGQGTSVTVSS

SEQ ID NO: 31 - protein, 121 aa, Artificial
  nanobody clone
EVQLQQSGPELVKPGASVKISCKASGYTFTDYYMKWVKQRHGQSLEWIGNINPYNGDTNYSQNFKGKATLTVDKSSTTAYMELRSLTSEDSAVYYCARLEDGYYGYAVDYWGQGTSVTVSS

SEQ ID NO: 32 - protein, 121 aa, Artificial
  nanobody clone
EVQLQQSGPELVKPGASVKISCKASGYTFTDYYMKWAKQSHGKSLEWIGHINPVNGDTSYNQKFKGKATLTVDKSSSTAVMELRSLTSEDSAVYYCARLEDGYYGYTMDYWGQGTSVTVSS

SEQ ID NO: 33 - protein, 121 aa, Artificial
  nanobody clone
EVQLQQSGPELVKPGASVKISCKASGYTFTDYYMKWVKQSHGKSLEWIGDINPNNGGARYNQKFKGRATLTVDKSSSTAYMELRSLTSEDSAVYYCSRLEDGYYGYAVDYWGQGTSVTVSS

SEQ ID NO: 34 - protein, 121 aa, Artificial
  nanobody clone
EVQLQQSGPELVKPGASVKISCKASGYTFTDYYMKWEKQSHGKSLEWIGDINPNNGGTRYNQKFRGKATLTVDKSSSTAYMELRSLTSEDSAVYYCARLEDDYYGYAVDYWGQGTSVTVSS

SEQ ID NO: 35 - protein, 121 aa, Artificial
  nanobody clone
EVQLQQSGPELVKPGASVKISCKASGYTFTDYYMKWVKQSHGKSLEWIGDINPKNGGTNYNQKFKDKATLTVDKSSSTAYMELRSLTSEDSAVYYCARLEDGYYGYAIDYWSQGTSVTVSS

SEQ ID NO: 36 - DNA, 47 bp, Artificial
  human-mouse chimeric IgG1(delta)CH1 transcript sequence
gacaatggtcaccgtctcttcagtgcccagggattgtggttgtaagc

SEQ ID NO: 37 - DNA, 52 bp, Artificial
  synthetic primer
  residues (1)..(47): = DNA
  misc_feature (36)..(47): n is a, c, g, t or u
  residues (48)..(52): = RNA
ctacactctttccctacacgacgctcttccgatctnnnnnnnnnnnnggggg

SEQ ID NO: 38 - DNA, 35 bp, Artificial
  synthetic primer
ctacactctttccctacacgacgctcttccgatct

SEQ ID NO: 39 - DNA, 22 bp, Artificial
  synthetic primer
ggtggttgtgcaggccctcatg

SEQ ID NO: 40 - DNA, 23 bp, Artificial
  synthetic primer
ccatggagttagtttgggcagca

SEQ ID NO: 41 - DNA, 23 bp, Artificial
  synthetic primer
caaggcttacaaccacaatccct

SEQ ID NO: 42 - DNA, 200 bp, Artificial
  Sequence of Singularity Minotaur array
gacgcctggaccagggcctgcgtgggaaaggcctctgggcacactcaggggctttttgtgaagggtcctcctactgtggtagttatagtggttatggttatggttatagttatggttatacccacagtgatgaacccagcagcaaaaactgaccggactcccaaggtttatgcacacttctccgctcagagctctccagg

SEQ ID NO: 43 - DNA, 198 bp, Artificial
  Sequence of Singularity Minotaur array
ctattccctgggaagctcctcctgacagccccgcctccagttccaggtgtggttattgtcagggggtgtcagactgtgatgatacgataggtgtggttttagttattgtagtgttgctaccacagtggtgctgcccatagcagcaaccaggccaagtagacaggcccctgctgtgcagccccaggcctccagctcacc

SEQ ID NO: 44 - DNA, 304 bp, Artificial
  Sequence of Singularity Minotaur array
gaccaagttgtgctgagcccagcaagggaaggtccccaaacaaaccaggaagtttctgaaggtgtctgtgtcacagtggtagttgtcctgatggttatagttatggttatggttgtggttatggttatggttgtagtggttatgattgttatggttatggtggttatggtggttatggtggttatggttatagtagttatagttatagttatacttacgaatataccacagtgacactcgccaggccagaaaccccatcccaagtcagcggaatgcagagagagcagggaggacatgtttagga

SEQ ID NO: 45 - DNA, 275 bp, Artificial
  Sequence of Singularity Minotaur array
cacaagcccagcccccacccaggaggccccagagcacagggcgccccgtcggattctgaacagccccgagtcacagtgagactatcgtgatgatggttactgctacacccacagtgactcaggccctgacataaagtctgacccgcacacaggtgtggagctggccaatgcatccccaggggcactgggctcccaagcactgtgagaaaagcttcgtccaaaacggtctcctggccacagtcggaggccccgccagagaggggagcagccacccc

SEQ ID NO: 46 - DNA, 223 bp, Artificial
  Sequence of Singularity Minotaur array
caaatgcctggaccagggcctgcgtgggaaaggtctctggccacactcgggctttttgtgaagggccctcctgctgtggtagttatggtggttatggttatggtggttatggttgttatggttatggttatggttatggttataccatagtgatgaacccagtggcaaaaactggctggaaacccaggggctgtgtgcacgcctcagcttggagctctccagg

SEQ ID NO: 47 - DNA, 229 bp, Artificial
  Sequence of Singularity Minotaur array
ctgttccctgggaagctcctcctgacagccccgcctccagttccaggtgtggttattgtcaggcgatgtcagactgtggtagttatggtggttatggttatggtggttatggttgttatggttatggttatggttatggttatggttataccacagtggtgccgcccatagcagcaaccaggccaagtagacaggcccctgctgcgcagccccaggcatccacttcacc

SEQ ID NO: 48 - DNA, 214 bp, Artificial
  Sequence of Singularity Minotaur array
aaccaaggggtgttgagcccagcaagggaaggcccccaaacagaccaggaggtttctgaaggtgtctgtgtcacagtggtagttgttatagtggttatggttatggttatggttgtggttatggttatggttataccacagtgacactcacccagccagaaaccccattccaagtcagcggaagcagagagagcagggaggacacgtttaggat

SEQ ID NO: 49 - DNA, 202 bp, Artificial
  Sequence of Singularity Minotaur array
gttacgatttgacgcctggaccagggcctgcgtgggaaaggccgctgggcacactcaggggctttttgtgaaggcccctcctactgtggtattgtggtagctattgtggtagttattatggtaccacagtgatgaaactagcagcaaaaactggccggacacccagggaccatgcacacttctcagcttggagctctccagg

SEQ ID NO: 50 - DNA, 787 bp, Artificial
  Sequence of Singularity Sapacos array
gggccctgcctctgagctcctctttgcatccaatctgctgaagaacatggctctagggaaacccagttgtagacctgagggccccggctcttcaatgagccatctccgtcccggggccttatatcagcaagtgacgcacacaggcaaatgccagggtgtggtttcctgtttaaatgtagcctcccccgctgcagaactgcagagcctgctgaattctggctgaccagggcagtcaccagagctccagacaatgtctgtctccttcctcatcttcctgcccgtgctgggcctcccatggggtcagtgtcagggagatgccgtattcacagcagcattcacagactgaggggtgtttcactttgctgtttccttttgtctccaggtgtcctgtcacaggtgcagctggtggagtcaggtggaggattagttcaggctggaggttctcttcgactatcctgcgcggccagtgggcgcaccttcagctcctatgccatgggctggtttcgtcaagcacctgggaaggagagggaatttgtggcagccatttcctggtcaggtggcagcacatactatgcagactctgtaaaagggcggtttaccataagtagagacaatgccaagaacactgtgtacctgcagatgaacagcttgaaaccagaagatacagctgtctattactgtgctgctcacagtgaggggaagtcagtgtgagcccagacacaaacctccctgcagggatgctcaggaccccagaaggcacccagcactaccagcgcagggcccagac

SEQ ID NO: 51 - DNA, 783 bp, Artificial
  Sequence of Singularity Sapacos array
ggggacacacatcattaaacaaggattgggacagggacttcagcgtcccactgttgcatggcccataaattatgtgtgttctctttctcatcttggatcaagtctagagctatgaaatagtatccctcatgaatatgcaaataacctgagatttactgaagtaaatacagatctgtcctgtgccctgagagcatcacccagcaaccacatctgtcctctagagaatcccctgagagctccgttcctcaccatggactggacctggaggatcctcttcttggtggcagcagccacaggtaagaggctccctagtcccagtgatgagaaagagattgagtccagtccagggagatctcatccacttctgtgttctctccacaggagcccactcccaggtgcagctggtggagagcgggggtgggttagtacaaccaggtggctccctacgacttagttgcgctgcaagtggcagcatcttctccataaacgccatgggctggtaccggcaagcccctgggaagcagcgtgaactggtggcagccatcacttctggaggctctacgaactatgcagactccgttaaaggacgcttcaccatttcaagagataatgccaagaacacagtgtatctgcagatgaattcattgaaacccgaggatactgcagtctactactgtaatgctcacagtgtgaaaacccacatcctgagggtgtcagaaaccccagggaggaggcagctgtgctggggctgagaaatgaaagggattactatttttaatgttg

SEQ ID NO: 52 - DNA, 783 bp, Artificial
  Sequence of Singularity Sapacos array
gggcatggctagttgaggccccaggaagagaactgagttctcaaagggcaaagcaagcatcctcatcccagggcgagcctaaaagactggggcctccctcatcccttttcacctctttatacaaaggcaccacctacatgcaaatcctcacttaggcacccacaggaaaccaccacacatttccttaaattcagggtccagctcacatgggaaatactttctgagagtcatggacctcctgcacaagaacatgaaacacctgtggttcttcctcctcctggtggcagctcccagatgtgagtgtctcaaggctgcagacatgggggtatgggaggtgcctctgatcccagggctcactgtgggtctctctgttcacaggggtcctgtctcaggtgcagctggtggagtctggtgggggcctcgtccaaccaggaggctcactccgactttcctgtgctgcttcaggatcgatctttagtataaatgccatgggctggtacaggcaggcccctgggaagcagagagagctggttgctgccatcaacactggaggtggcagcacctattatgcagattctgtgaaagggcgttttaccatttcccgggacaatgcaaagaacacactgtatctacaaatgaacagcttaaaaagtgaaggaacagctgtctactactgcgcagcccacagtgaggggaggtgagtgtgagcccagacacaaacctccctgcagggaggcggaggggaccggcgcaggtgctgctcagagccagcagggggcgcgc

SEQ ID NO: 53 - DNA, 784 bp, Artificial
  Sequence of Singularity Sapacos array
tgacttctgcaaagacttctactcagaatctacttgcccagccttagattaatgccatctgaattacactgatcatgttactatcactgctcctcaccacagatgcaacaccctcctgagtcctgaaacctgactccatcccatagagtagggcacagatgaggggaatgcaaatctccaccagctccaccctcctctgggttgaaaaagccgagcacaggtcccagctcagtgactcctgtgccccaccatggacacactttgctccacgctcctgctgctgaccatcccttcatgtgagtgctgtggtcagggactccttcacgggtgaaacatcagttttcttgtttgtgggcttcatcttcttatgctttctccacaggggtcttgtcccaagtccagctggtggagtctggtggtggccttgtacaggcaggagggtcacttcggcacagctgtgctgcgtctggactcacctttggatcctatgccatgggctggtacaggcaggcccccgggaaagaacgagagctggttgctgccatcagcagtgggggcagtacgtattatgcagactctgtcaaaggccgcttcaccatatccagagataatgccaagaacactctctacctgcagatgaacagcctgaagcctgaaggaacagctgtgtactactgcaatgcacacaaagacacagcccagggcacctcctgtacaaaaacccaggctgcttctcattggtgctccctccccacctctgcagaacaggaaagtgcagctgaga

SEQ ID NO: 54 - DNA, 801 bp, Artificial
  Sequence of Singularity Sapacos array
acagcatattttccaaataccatcattgtcagcaaacttctgcagagcaccgtcttcttatatgggtacagcctattcctccagcatcccactagagcttcttatatagtaggagacatgcaaatagggccctccctctactgatgaaaaccaacccaaccctgaccctgcaggtctcagagaggagccttagccctggactccaaggcctttccacttggtgatcagcactgagcacagaggactcaccatggagttggggctgagctgggttttccttgttgctattttagaaggtgattcatggaaaactaggaagattgagtgtgtgtggatatgagtgtgagaaacagtggatttgtgtggcagtttctgaccttggtgtctctttgtttgcaggtgtccagtgtcaggtgcagctggtggagtcaggaggtggtttggttcaagcagggggcagtttacgacacagctgtgctgcttctgggctcacttttggaagctatgccatgggctggtatcggcaagcgccagggaaagaaagagaacttgttgcagccataagcagtggtggctcaacctactatgcagactctgtgaaaggacgcttcaccatctccagggataatgccaagaacacagtctacctgcagatgaacagcctgaagcctgaaggaacagctgtgtcctactgcaatgctcacagtgaggggaagtcagtgtgagcccagacacaaacctccctgcaggggtcccttgggaccaccagggggcgacagggcattgagcacggggctgtct

SEQ ID NO: 55 - DNA, 748 bp, Artificial
  Sequence of Singularity Savnars array
gggccctgcctctgagctcctctttgcatccaatctgctgaagaacatggctctagggaaacccagttgtagacctgagggccccggctcttcaatgagccatctccgtcccggggccttatatcagcaagtgacgcacacaggcaaatgccagggtgtggtttcctgtttaaatgtagcctcccccgctgcagaactgcagagcctgctgaattctggctgaccagggcagtcaccagagctccagacaatgtctgtctccttcctcatcttcctgcccgtgctgggcctcccatggggtcagtgtcagggagatgccgtattcacagcagcattcacagactgaggggtgtttcactttgctgtttccttttgtctccaggtgtcctgtcagccagggtggaccagacacctcggagtgttaccaaggagacaggagaatcactcaccatcaactgtgtgcttcgagatgcttcctatgcattgggttctacgtgctggtacaggaagaagtctgggtcaacaaatgaagagagcataagcaaaggaggcagatatgtggagactgtcaacagcggctccaaaagtttctccctgagaattaatgacctgactgtagaagatggtggcacctaccgctgtggggtccacagtgaggggaagtcagtgtgagcccagacacaaacctccctgcagggatgctcaggaccccagaaggcacccagcactaccagcgcagggcccagac

SEQ ID NO: 56 - DNA, 765 bp, Artificial
  Sequence of Singularity Savnars array
agcacaatttcccaatgctttcaatatcacagatctccccgaggacattctgacatgctctgagccccactatctccaaaggcctctcaccccagagcttactatatagtaggagatatgcaaatagagccctccgtctgctgatgaaaaccagcccagccctgaccctgcagctctgagagaggagcccagccctgggattttcaggtgttttcatttggtgatcaggactgaacagagagaactcaccatggagtttgggctgagctggctttttcttgtggctattttaaaaggtaattcatggagaaatagaaaaattgagtgtgaatggataagagtgagagaaacagtggatacgtgtggcagtttctgaccagggtttctttttgtttgcaggtgtccagtgtgctcgagtagaccagactcctaagactatcaccaaggagacaggagaatcactcaccatcaactgtgttcttagagatacgtcctatgcattggggtccacctactggtacaggaagaagctgggctctacaaatgaggaaagcatttcaaaaggtgggcggtatgtggagactgtcaacagtggaagcaaaagtctatctctgcgtataaatggcctgaaggtggaagacagctggacataccgctgcaaagccacagtgaggggaagtcattgtgagcccagacacaaacctccctgcaggaacgatgggggggaaatcagcggcagggggcgctcaggacccgctgatcaga

SEQ ID NO: 57 - DNA, 3162 bp, Artificial
  Sequence of Longhorn VDJ
tgtcttttctgtttttaattttattccagtaaattcattttcctcctttggagactggctaaagacttatggttttgtttgtctttgaaaaaacgacaatgacaacaacaacaccagctgttgatttcactgatcgttgttgctgttttgtgtgtctgtgttcatctcaatattattaatttctgcacttttttgtcatttccttcctactactgcctctgtgcttttgtggtgttctgttctaatttctgtaggtagtaagttggattctttccttccatttcttgaagaaggcctgtatcactgtcagctttcgtttcagaactgctgggccggcatagagtttagagataagagtgtgttagtcagtcagtcgtgtctgacactttgttgccccagagactgtagctcgccagtcttctttgtcctttctaggcaagaatcctggagtggcctgccatttcctactccagggaatctcacacctggggagtcaacccaggtctcctacattgcaagtggattctttaccacaggaggcaaaattctcattatctacagaacactgtagatagaaaaccctaaagactccacacaaagctattagaccacactcatccagagagcactgaactcagtaagtcagagtgggtaccagcatctgattgctcatggcttaggagttgttggggattgtctggatcccacgtctgatgccataaactaaaccccatagaaaccaactggtagtggatgtggggatgtgtctgcagccttccagtcccgtgagatgagaagcatatttcggttttacaggtggtccagtgggatcacgagatcagagaaactgctgaagagtggaaagcctgttaggcgtgcaccacaagactccactgggtttgatgatggatgaaggtgttgagccaagggatgaggacacatgtaggagctcagtgacctcgatgccagaggccacagttccagggtcaccctcaggagtcttgaccatgttgcatggcatcgggcacaggtgaccccacaagctctgaggcagagtgaggtggggcctctccccagagcacattcaggtgtcaggaaactggatctggcacaaaagacatgctcatgcagaactgctgtgaccaaagccagactgggaaggccttttcatagacagtaggttctcagtcagtgtacaggtcagatccctgtgccgaggaagagctgacctgcaccagacccgaaggggtgacatctcagggcaggagaagaccagggtcacatggacagggcgtcagtcagagtggggacacacactggtcttttttgctccaggagggataacaccactgatgacatgatgacagcactccccaccccgggcaaacccgctgtaaatccacacatgctgtgactgtcgtcagccccagtctacatcctcacacacagaaatgcaggtgtgaggccacagccgcagggccacagggagatgggggagcggagccctaaggagctcccaggagctgtctgtccccactcctcagcagacttggggtcctcagtgtccaccctcagggaagagatgctgaggttcacgagttcctgcagacactcagttgtgcacaggaaaatgcagcccactgggcaagtcagccgtgctgggaagacgagcatctcagcacaaatgtctactgtgttcacagaattaatactgcgcttaattactatgaacaaacctcctctagattgattcttgaggtgtttaagctgttcaagatgaaaaaacagtcaagaaagcaaaggaaaatataaatgctttaggaattctgatagacattaaattgtactcaactccacttgaattcagtgtgattattacttgtatatttttctctgatgtttgtatatggagtaagtaaacatcagctaatgagaaaagaaaaaaaaaataagtttgtgtctaacaaatggctggtacctgaatgtgtggtgggcatttgttgagtaacagaaaacacattcgtgggattaaagtctcttccccaaatctgccattttccctggaagcatttttttcctgaccagcccttcttcatcatttcattcctcaaagtcttcctggtgtggggagggggatcccaggaagggctgagttctctgaggccacagacagcaccccctcacagggggagcccaacacacaggacctctattcactgctttctgctttttatacagaggtccctcctgtatgcaaatatccactcaagtcacagggtagaaacacagcaccagctcccttaaattcaggctcctcctgaggcttgaagagacttgtgggagtggtgactctcatctgctccaagatgaacccactgtggaccctcctctttgtgctctcagcccccagaggtgagtgtctctgggtcagacataggcacgtggggaagctgcctctgagcccacgggtcaccgtgcttctctctctccacaggggtcctgtcccaggtgcagctgcgggagtcgggccccagcctggtgaagccgtcacagaccctctccctcacctgcacggtctctggattctcattgagcgacaaggctgtaggctgggtccgccaggctccagggaaggcgctggagtggctcggtggtatagacactggtggaagcacaggctataacccaggcctgaaatcccggctcagcatcaccaaggacaactccaagagccaagtctctctgtcagtgagcagcgtgacaactgaggactcggccacatactactgtactactgtgcaccagagttgtcctgatggttatagttatggttatggttgtggttatggttatggttgtagtggttatgattgttatggttatggtggttatggtggttatggtggttatggttatagtagttatagttatagttatacttacgaatattacgtcgatgcctggggccaaggactcctggtcaccgtctcctcaggtgagtcctcaacagccctctctcctcactctctctcagggttttggtgcactttggggaaaatcgagggtgtcgggtctaaggggcctggggcagccgggggtctgaaacactgagggcccaggggcccaggcttacagcaccgaggagcagaggctccaggcacc